(19) United States
(12) Patent Application Publication
(10) Pub. No.: US 2023/0391764 A1
(43) Pub. Date: Dec. 7, 2023

(54) BENZOTHIAZOLYL BICYCLO[1.1.1]PENTANE DERIVATIVES FOR THE TREATMENT AND PROPHYLAXIS OF HEPATITIS B VIRUS INFECTION

(71) Applicant: Hoffmann-La Roche Inc., Little Falls, NJ (US)

(72) Inventors: Hongying YUN, Shanghai (CN); Bo ZHANG, Shanghai (CN); Xiufang ZHENG, Shanghai (CN)

(73) Assignee: Hoffmann-La Roche Inc., Little Falls, NJ (US)

(21) Appl. No.: 18/454,315

(22) Filed: Aug. 23, 2023

Related U.S. Application Data
(63) Continuation of application No. PCT/EP2022/054316, filed on Feb. 22, 2022.

(30) Foreign Application Priority Data
Feb. 24, 2021 (WO) PCT/CN2021/077651

(51) Int. Cl. C07D 417/12; A61P 31/14

(52) U.S. Cl. CPC C07D 417/12; A61P 31/14

(57) ABSTRACT

The present invention provides novel compounds having the general formula:

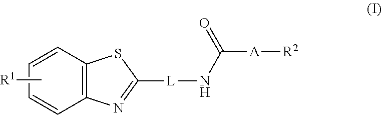

wherein R¹, R², L, and A are as described herein, or a pharmaceutically acceptable salt thereof, compositions including the compounds and methods of using the compounds.

### CROSS-REFERENCE TO PRIOR APPLICATIONS

This application is a continuation of International Application No. PCT/EP2022/054316 having an International Filing Date of Feb. 22, 2022 and which claims benefit under 35 U.S.C. § 119 to International Application No. PCT/CN2021/077651 having an International Filing Date of Feb. 24, 2021. The entire contents of both are incorporated herein by reference.

### FIELD OF THE INVENTION

The present invention relates to organic compounds useful for therapy and/or prophylaxis of HBV infection in a mammal, and in particular to HBsAg (HBV Surface antigen) and HBeAg (HBV e antigen) inhibitors useful for treating HBV infection. In particular, the present invention relates to benzothiazolyl bicyclo[1.1.1]pentane derivatives that have anti-virus activity, as well as their manufacture, pharmaceutical compositions containing them and their potential use as medicaments.

### BACKGROUND OF THE INVENTION

Hepatitis B virus (HBV) is one of the most dangerous human pathogens. A safe and effective vaccine has been available for longer than two decades; however, WHO estimated that approximately 257 million people are chronically infected with HBV. Chronic Hepatitis B (CHB) infection predisposes its host to severe liver disease, including liver cirrhosis and hepatocellular carcinoma, if left untreated. HBV infection is ranked among the top unmet medical need worldwide. The currently approved drugs have contributed to substantial progress in CHB treatment; however, the cure rate remains less than 10%.

The control of viral infection needs an effective immune surveillance. Upon recognition of viral infection, the host innate immune system could respond within minutes to impede viral replication and limits the development of a chronic and persistent infection. The secretion of antiviral cytokines from infected hepatocytes and intra-hepatic immune cells is critically important for the clearance of viral infection. However, chronically infected patients only display a weak immune response due to various escape strategies adopted by the virus to counteract the host cell recognition systems and the subsequent antiviral responses.

Many observations showed that several HBV viral proteins could counteract the initial host cellular response by interfering with the viral recognition signaling system and subsequently the interferon (IFN) antiviral activity. Among these, the excessive secretion of HBV empty subviral particles (SVPs, HBsAg) may contribute to immune tolerant state observed in CHB patients. The persistent exposure to HBsAg and other viral antigens can lead to HBV-specific T-cell functional impairment and depletion (Kondo et al. Journal of Immunology (1993), 150, 4659-4671; Kondo et al. Journal of Medical Virology (2004), 74, 425-433; Fisicaro et al. Gastroenterology, (2010), 138, 682-693). Moreover, HBsAg has been reported to suppress immune cell functions, including monocytes, dendritic cells (DCs) and natural killer (NK) cells (Op den Brouw et al. Immunology, (2009b), 126, 280-289; Woltman et al. PLoS One, (2011), 6, e15324; Shi et al. J Viral Hepat. (2012), 19, e26-33; Kondo et al. ISRN Gasteroenterology, (2013), Article ID 935295).

HBsAg is an important biomarker for prognosis and treatment response in CHB. However, the achievement of HBsAg loss and seroconversion is rarely achieved in CHB patients. HBsAg loss with or without anti-HBsAg seroconversion remains the ideal clinical treatment endpoints. Current therapies, such as nucleos(t)ide analogues, are effective in suppressing HBV DNA, but are not effective in reducing HBsAg level. Nucleos(t)ide analogues, even with prolonged therapy, have demonstrated HBsAg clearance rates comparable to those observed naturally (Janssen et al. Lancet. (2005), 365, 123-129; Marcellin et al. N. Engl. J. Med., (2004), 351, 1206-1217; Buster et al. Hepatology (2007), 46, 388-394). Therefore, there is an urgent need for the development of novel therapeutic agents that could efficiently reduce HBsAg (Wieland, S. F. & F. V. Chisari. J Virol, (2005), 79, 9369-9380; Kumar et al. J Virol, (2011), 85, 987-995; Woltman et al. PLoS One, (2011), 6, e15324; Op den Brouw et al. Immunology, (2009b), 126, 280-289).

### SUMMARY OF THE INVENTION

Objects of the present invention are novel compounds of formula (I), their manufacture, medicaments based on a compound in accordance with the invention and their production as well as the use of compounds of formula (I) as HBV inhibitors and for the treatment or prophylaxis of HBV infection. The compounds of formula (I) show superior anti-HBV activity. In addition, the compounds of formula (I) also show good safety and good PK profiles.

One aspect of the invention pertains to a compound of formula (I),

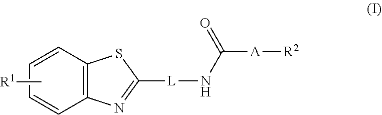

wherein
R¹ is hydrogen, halogen, C₂₋₆alkynyl, cyano, morpholinyl, or C₁₋₆alkoxyC₁₋₆alkoxy;
L is a C₅₋₁₂cycloalkyl, wherein L is a monocyclic ring or a bicyclic ring, and wherein the bicyclic ring is a bridged, spiro or fused ring;
A is a 5 or 6 membered heteroaryl containing one to three heteroatoms independently selected from N, O, and S;
R² is C₁₋₆alkylsulfonylC₁₋₆alkyl, C₁₋₆alkylsulfonylC₃₋₇cycloalkyl, halo(C₁₋₆alkylsulfonyl)C₁₋₆alkyl, carbamoyl(C₁₋₆alkylsulfonyl)C₁₋₆alkyl, C₁₋₆alkylsulfonyl, C₁₋₆alkoxysulfonyl, (C₁₋₆alkoxyC₁₋₆alkyl)sulfonyl, C₃₋₇cycloalkylsulfonyl, C₁₋₆alkylsulfinylC₁₋₆alkyl, C₁₋₆alkylsulfinyl, C₃₋₇cycloalkylC₁₋₆alkylsulfinyl, C₃₋₇cycloalkylsulfinyl, C₁₋₆alkylsulfonimidoyl, C₃₋₇cycloalkylC₁₋₆alkylsulfonimidoyl, C₃₋₇cycloalkylsulfonimidoyl, C₁₋₆alkylsulfanyl, C₁₋₆alkylsulfanylC₁₋₆alkyl, haloC₁₋₆alkyl, haloC₁₋₆alkylsulfonylC₁₋₆alkyl, C₃₋₇cycloalkylC₁₋₆alkylsulfonyl, or (C₁₋₆ alkylcarbonyl)sulfamoyl;
or a pharmaceutically acceptable salt thereof.

Another aspect of the invention pertains to a process for the preparation of a compound of formula (I), as well as a compound of formula (I) or a pharmaceutically acceptable salt thereof when manufactured according to the process.

Another aspect of the invention pertains to a pharmaceutical composition comprising a compound of formula (I) or a pharmaceutically acceptable salt thereof, and a pharmaceutically acceptable excipient.

Another aspect of the invention pertains to a compound of formula (I) or a pharmaceutically acceptable salt thereof for use as therapeutically active substance.

Another aspect of the invention pertains to a compound of formula (I) or a pharmaceutically acceptable salt thereof for use in the treatment or prophylaxis of HBV infection.

Another aspect of the invention pertains to the use of a compound of formula (I) or a pharmaceutically acceptable salt thereof for the treatment or prophylaxis of HBV infection.

Another aspect of the invention pertains to the use of a compound of formula (I) or a pharmaceutically acceptable salt thereof for the inhibition of HBsAg.

Another aspect of the invention pertains to the use of a compound of formula (I) or a pharmaceutically acceptable salt thereof for the inhibition of HBeAg.

Another aspect of the invention pertains to the use of a compound of formula (I) or a pharmaceutically acceptable salt thereof for the preparation of a medicament for the treatment or prophylaxis of HBV infection.

Another aspect of the invention pertains to a method for the treatment or prophylaxis of HBV infection, which method comprises administering an effective amount of a compound of formula (I) or a pharmaceutically acceptable salt thereof.

### DETAILED DESCRIPTION OF THE INVENTION

### Definitions

Unless otherwise defined, all technical and scientific terms used herein have the same meaning as commonly understood by one of ordinary skill in the art to which this invention belongs. Furthermore, the following definitions are set forth to illustrate and define the meaning and scope of the various terms used to describe the invention.

The nomenclature used in this application is based on IUPAC systematic nomenclature, unless indicated otherwise.

The term “chiral” denotes the ability of non-superimposability with the mirror image, while the term “achiral” refers to embodiments which are superimposable with their mirror image. Chiral molecules are optically active, i.e., they have the ability to rotate the plane of plane-polarized light. Whenever a chiral center is present in a chemical structure, it is intended that all stereoisomers associated with that chiral center are encompassed by the present invention.

The term “compound(s) of this invention” and “compound(s) of the present invention” refers to compounds of formula (I) and stereoisomers, solvates or salts thereof (e.g., pharmaceutically acceptable salts).

The term “substituent” denotes an atom or a group of atoms replacing a hydrogen atom on the parent molecule.

As used herein, the term “C₁₋₆alkyl” alone or in combination signifies a saturated, linear- or branched chain alkyl group containing 1 to 6, particularly 2 to 6 or 1 to 4 carbon atoms, for example methyl, ethyl, propyl, isopropyl, butyl, isobutyl, tert-butyl and the like. Particular “C₁₋₆alkyl” groups are methyl and ethyl.

The term “C₁₋₆alkoxy” alone or in combination signifies a group C₁₋₆alkyl-O—, wherein the “C₁₋₆alkyl” is as defined above; for example methoxy, ethoxy, propoxy, iso-propoxy, n-butoxy, iso-butoxy, 2-butoxy, tert-butoxy, pentoxy, hexyloxy and the like. Particular “C₁₋₆alkoxy” groups are methoxy and ethoxy and propoxy.

The term “C₃₋₇cycloalkyl” denotes to a saturated carbon ring containing from 3 to 7 carbon atoms, particularly from 3 to 6 carbon atoms, for example, cyclopropyl, cyclobutyl, cyclopentyl, cyclohexyl, cycloheptyl and the like. Particular “C₃₋₇cycloalkyl” group is cyclopropyl. The term “C₅₋₁₂cycloalkyl” denotes to a saturated carbon ring containing from 5 to 12 carbon atoms, for example, bicyclo[1.1.1]pentanyl.

The term “halogen” or “Halo” denotes fluoro, chloro, bromo, or iodo.

The term “haloC₁₋₆alkyl” denotes an alkyl group wherein at least one of the hydrogen atoms of the alkyl group is replaced by same or different halogen atoms, particularly fluoro atoms. Examples of haloC₁₋₆alkyl include monochloro-, difluoro- or trifluoro-methyl, -ethyl or -propyl, for example difluoromethyl.

The term “carbonyl” alone or in combination refers to the group —C(O)—.

The term “heteroaryl” denotes a monovalent aromatic heterocyclic mono- or bicyclic ring system of 5 to 12 ring atoms, comprising 1, 2, 3 or 4 heteroatoms selected from N, O and S, the remaining ring atoms being carbon. Examples of heteroaryl moieties include, but not limited to, pyrrolyl, furanyl, thienyl, imidazolyl, oxazolyl, thiazolyl, triazolyl, oxadiazolyl, thiadiazolyl, tetrazolyl, pyridinyl, pyrazinyl, pyrazolyl, pyridazinyl, pyrimidinyl, triazinyl, isoxazolyl, benzofuranyl, isothiazolyl, benzothienyl, indolyl, isoindolyl, isobenzofuranyl, benzimidazolyl, benzoxazolyl, benzoisoxazolyl, benzothiazolyl, benzoisothiazolyl, benzooxadiazolyl, benzothiadiazolyl, benzotriazolyl, purinyl, quinolinyl, isoquinolinyl, quinazolinyl or quinoxalinyl. Heteroaryl can be further substituted by halogen, C₁₋₆alkyl, haloC₁₋₆alkyl, cyano, C₃₋₇cycloalkyl, (C₁₋₆alkyl)₂amino or C₁₋₆alkoxy.

The term “sulfonyl” alone or in combination refers to the group —S(O)₂—.

The term “sulfinyl” alone or in combination refers to the group —SO—.

The term “sulfanyl” alone or in combination refers to the group —S—.

The compounds according to the present invention may exist in the form of their pharmaceutically acceptable salts. The term “pharmaceutically acceptable salt” refers to conventional acid-addition salts or base-addition salts that retain the biological effectiveness and properties of the compounds of formula (I) and are formed from suitable non-toxic organic or inorganic acids or organic or inorganic bases. Acid-addition salts include for example those derived from inorganic acids such as hydrochloric acid, hydrobromic acid, hydroiodic acid, sulfuric acid, sulfamic acid, phosphoric acid and nitric acid, and those derived from organic acids such as p-toluenesulfonic acid, salicylic acid, methanesulfonic acid, oxalic acid, succinic acid, citric acid, malic acid, lactic acid, fumaric acid, and the like. Base-addition salts include those derived from ammonium, potassium, sodium and, quaternary ammonium hydroxides, such as for example, tetramethyl ammonium hydroxide. The chemical modification of a pharmaceutical compound into a salt is a technique well known to pharmaceutical chemists in order to obtain improved physical and chemical stability, hygroscopicity, flowability and solubility of compounds. It is for example described in Bastin R. J., et al., Organic Process Research & Development 2000, 4, 427-435. Particular are the sodium salts of the compounds of formula (I).

The term “therapeutically effective amount” denotes an amount of a compound or molecule of the present invention that, when administered to a subject, (i) treats or prevents the particular disease, condition or disorder, (ii) attenuates, ameliorates or eliminates one or more symptoms of the particular disease, condition, or disorder, or (iii) prevents or delays the onset of one or more symptoms of the particular disease, condition or disorder described herein. The therapeutically effective amount will vary depending on the compound, the disease state being treated, the severity of the disease treated, the age and relative health of the subject, the route and form of administration, the judgement of the attending medical or veterinary practitioner, and other factors.

The term “pharmaceutical composition” denotes a mixture or solution comprising a therapeutically effective amount of an active pharmaceutical ingredient together with pharmaceutically acceptable excipients to be administered to a mammal, e.g., a human in need thereof.

### HBV Inhibitors

The present invention relates to (i) a compound of formula (I),

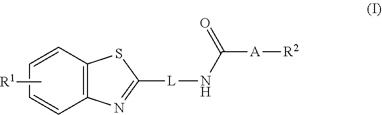

wherein
R¹ is hydrogen, halogen, C₂₋₆alkynyl, cyano, morpholinyl, or C₁₋₆alkoxyC₁₋₆alkoxy;
L is a C₅₋₁₂cycloalkyl, wherein L is a monocyclic ring or a bicyclic ring, and wherein the bicyclic ring is a bridged, spiro or fused ring;
A is a 5 or 6 membered heteroaryl containing one to three heteroatoms independently selected from N, O, and S;
R² is C₁₋₆alkylsulfonylC₁₋₆alkyl, C₁₋₆alkylsulfonylC₃₋₇cycloalkyl, halo(C₁₋₆alkylsulfonyl)C₁₋₆alkyl, carbamoyl(C₁₋₆alkylsulfonyl)C₁₋₆alkyl, C₁₋₆alkylsulfonyl, C₁₋₆alkoxysulfonyl, (C₁₋₆alkoxyC₁₋₆alkyl)sulfonyl, C₃₋₇cycloalkylsulfonyl, C₁₋₆alkylsulfinylC₁₋₆alkyl, C₁₋₆alkylsulfinyl, C₃₋₇cycloalkylC₁₋₆alkylsulfinyl, C₃₋₇cycloalkylsulfinyl, C₁₋₆alkylsulfonimidoyl, C₃₋₇cycloalkylC₁₋₆alkylsulfonimidoyl, C₃₋₇cycloalkylsulfonimidoyl, C₁₋₆alkylsulfanyl, C₁₋₆alkylsulfanylC₁₋₆alkyl, haloC₁₋₆alkyl, haloC₁₋₆alkylsulfonylC₁₋₆alkyl, C₃₋₇cycloalkylC₁₋₆alkylsulfonyl, or (C₁₋₆alkylcarbonyl)sulfamoyl;
or a pharmaceutically acceptable salt thereof.

A further embodiment of present invention is (ii) the compound of formula (I), or a pharmaceutically acceptable salt thereof, according to (i), wherein R¹ is halogen or C₂₋₆alkynyl.

A further embodiment of present invention is (iii) the compound of formula (I), or a pharmaceutically acceptable salt thereof, according to (ii), wherein R¹ is fluoro, chloro, bromo, or ethynyl.

A further embodiment of present invention is (iv) the compound of formula (I), or a pharmaceutically acceptable salt thereof, according to according to any one of (i) to (iii), wherein L is

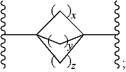

each of x, y, and z is independently an integer of 1, 2, or 3.

A further embodiment of present invention is (v) the compound of formula (I), or a pharmaceutically acceptable salt thereof, according to (iv), wherein L is

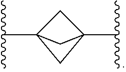 [SMILES: *C12CC(*)(C1)C2]

A further embodiment of present invention is (vi) the compound of formula (I), or a pharmaceutically acceptable salt thereof, according to according to any one of (i) to (v), wherein A is furanyl, oxadiazolyl, thiadiazolyl, oxazolyl, dihydrothiazolyl, thiazolyl, isothiazolyl, pyrazolyl, triazolyl, or pyridinyl.

A further embodiment of present invention is (vii) the compound of formula (I), or a pharmaceutically acceptable salt thereof, according to (vi), wherein

A is furanyl, thiadiazolyl, oxazolyl, or pyrazolyl.

A further embodiment of present invention is (viii) the compound of formula (I), or a pharmaceutically acceptable salt thereof, according to (vii), wherein

A is furanyl, 1,2,4-thiadiazolyl, oxazolyl, or pyrazolyl.

A further embodiment of present invention is (ix) the compound of formula (I), or a pharmaceutically acceptable salt thereof, according to according to any one of (i) to (viii), wherein R² is C₁₋₆alkylsulfonylC₃₋₇cycloalkyl, C₁₋₆alkylsulfonylC₁₋₆alkyl, haloC₁₋₆alkylsulfonylC₁₋₆alkyl, or C₁₋₆alkylsulfinylC₁₋₆alkyl.

A further embodiment of present invention is (x) the compound of formula (I), or a pharmaceutically acceptable salt thereof, according to (ix), wherein
R² is methylsulfonylcyclopropyl, 1-(methylsulfonyl)propyl, 2-(methylsulfonyl)propan-2-yl, 1-(methylsulfonyl)ethyl, bromo(methylsulfonyl)methyl, or 2-(methylsulfinyl)propan-2-yl.

A further embodiment of present invention is (xi) the compound of formula (I), or a pharmaceutically acceptable salt thereof, according to according to any one of (i) to (x), wherein
R¹ is halogen or C₂₋₆alkynyl;
L is

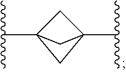 [SMILES: *C12CC(*)(C1)C2]

A is furanyl, thiadiazolyl, oxazolyl, or pyrazolyl; and
R² is C₁₋₆alkylsulfonylC₃₋₇cycloalkyl, C₁₋₆alkylsulfonylC₁₋₆alkyl, haloC₁₋₆alkylsulfonylC₁₋₆alkyl, or C₁₋₆alkylsulfinylC₁₋₆alkyl.

A further embodiment of present invention is (xii) the compound of formula (I), or a pharmaceutically acceptable salt thereof, according to (xi), wherein
R¹ is fluoro, chloro, bromo, or ethynyl;
L¹ is

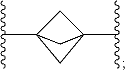 [SMILES: *C12CC(*)(C1)C2]

A is furanyl, 1,2,4-thiadiazolyl, oxazolyl, or pyrazolyl; and
   R² is methylsulfonylcyclopropyl, 1-(methylsulfonyl)propyl, 2-(methylsulfonyl)propan-2-yl, 1-(methylsulfonyl)ethyl, bromo(methylsulfonyl)methyl, or 2-(methylsulfinyl)propan-2-yl.

A further embodiment of present invention is (xiii) a compound selected from:
N-[3-(6-bromo-1,3-benzothiazol-2-yl)-1-bicyclo[1.1.1]pentanyl]-5-(methylsulfonylmethyl)furan-2-carboxamide;
N-[3-(6-bromo-1,3-benzothiazol-2-yl)-1-bicyclo[1.1.1]pentanyl]-5-(1-methylsulfonylcyclopropyl)furan-2-carboxamide;
N-[3-(6-bromo-1,3-benzothiazol-2-yl)-1-bicyclo[1.1.1]pentanyl]-5-(1-methylsulfonylcyclopropyl)-1,3,4-oxadiazole-2-carboxamide;
N-[3-(6-bromo-1,3-benzothiazol-2-yl)-1-bicyclo[1.1.1]pentanyl]-3-(1-methylsulfonylcyclopropyl)-1,2,4-thiadiazole-5-carboxamide;
N-[3-(5-bromo-1,3-benzothiazol-2-yl)-1-bicyclo[1.1.1]pentanyl]-5-(methylsulfonylmethyl)furan-2-carboxamide;
N-(3-(5-bromobenzo[d]thiazol-2-yl)bicyclo[1.1.1]pentan-1-yl)-5-(1-(methylsulfonyl)cyclopropyl)furan-2-carboxamide;
N-[3-(5-bromo-1,3-benzothiazol-2-yl)-1-bicyclo[1.1.1]pentanyl]-3-(1-methylsulfonylcyclopropyl)-1,2,4-thiadiazole-5-carboxamide;
N-[3-(6-cyano-1,3-benzothiazol-2-yl)-1-bicyclo[1.1.1]pentanyl]-5-(1-methylsulfonylcyclopropyl)furan-2-carboxamide;
N-[3-(5-cyano-1,3-benzothiazol-2-yl)-1-bicyclo[1.1.1]pentanyl]-5-(1-methylsulfonylcyclopropyl)furan-2-carboxamide;
N-[3-(6-ethynyl-1,3-benzothiazol-2-yl)-1-bicyclo[1.1.1]pentanyl]-5-(1-methylsulfonylcyclopropyl)furan-2-carboxamide;
N-[3-(6-fluoro-1,3-benzothiazol-2-yl)-1-bicyclo[1.1.1]pentanyl]-5-(1-methylsulfonylcyclopropyl)furan-2-carboxamide;
N-[3-(6-chloro-1,3-benzothiazol-2-yl)-1-bicyclo[1.1.1]pentanyl]-5-cyclopropylsulfonyl-furan-2-carboxamide;
N-[3-(6-chloro-1,3-benzothiazol-2-yl)-1-bicyclo[1.1.1]pentanyl]-5-(methylsulfonylmethyl)furan-2-carboxamide;
N-[3-(6-chloro-1,3-benzothiazol-2-yl)-1-bicyclo[1.1.1]pentanyl]-5-(1-methylsulfonylpropyl)furan-2-carboxamide;
N-(3-(6-chlorobenzo[d]thiazol-2-yl)bicyclo[1.1.1]pentan-1-yl)-5-(1-(methylsulfonyl)cyclopropyl)furan-2-carboxamide;
N-[3-(6-chloro-1,3-benzothiazol-2-yl)-1-bicyclo[1.1.1]pentanyl]-5-(1-methyl-1-methylsulfonyl-ethyl)furan-2-carboxamide;
N-[3-(6-chloro-1,3-benzothiazol-2-yl)-1-bicyclo[1.1.1]pentanyl]-5-(1-methylsulfonylethyl)furan-2-carboxamide;
5-[bromo(methylsulfonyl)methyl]-N-[3-(6-chloro-1,3-benzothiazol-2-yl)-1-bicyclo[1.1.1]pentanyl]furan-2-carboxamide;
5-(2-amino-1-methylsulfonyl-2-oxo-ethyl)-N-[3-(6-chloro-1,3-benzothiazol-2-yl)-1-bicyclo[1.1.1]pentanyl]furan-2-carboxamide;
N-[3-(6-chloro-1,3-benzothiazol-2-yl)-1-bicyclo[1.1.1]pentanyl]-2-(methylsulfonylmethyl)oxazole-5-carboxamide;
N-[3-(6-chloro-1,3-benzothiazol-2-yl)-1-bicyclo[1.1.1]pentanyl]-2-(1-methylsulfonylethyl)oxazole-5-carboxamide;
N-[3-(6-chloro-1,3-benzothiazol-2-yl)-1-bicyclo[1.1.1]pentanyl]-2-(1-methyl-1-methylsulfonyl-ethyl)oxazole-5-carboxamide;
N-[6-(5-chloro-1,3-benzoxazol-2-yl)spiro[3.3]heptan-2-yl]-2-(methylsulfamoylamino)pyridine-4-carboxamide;
N-[3-(6-chloro-1,3-benzothiazol-2-yl)-1-bicyclo[1.1.1]pentanyl]-2-(1-methylsulfonylcyclopropyl)thiazole-5-carboxamide;
N-[3-(6-chloro-1,3-benzothiazol-2-yl)-1-bicyclo[1.1.1]pentanyl]-2-(1-methyl-1-methylsulfonyl-ethyl)thiazole-5-carboxamide;
N-[3-(6-chloro-1,3-benzothiazol-2-yl)-1-bicyclo[1.1.1]pentanyl]-3-(methylsulfonylmethyl)isothiazole-5-carboxamide;
N-[3-(6-chloro-1,3-benzothiazol-2-yl)-1-bicyclo[1.1.1]pentanyl]-3-(1-methyl-1-methylsulfonyl-ethyl)isothiazole-5-carboxamide;
N-[3-(6-chloro-1,3-benzothiazol-2-yl)-1-bicyclo[1.1.1]pentanyl]-5-(1-methyl-1-methylsulfonyl-ethyl)isothiazole-3-carboxamide;
N-[3-(6-chloro-1,3-benzothiazol-2-yl)-1-bicyclo[1.1.1]pentanyl]-5-(1-methylsulfonylcyclopropyl)isothiazole-3-carboxamide;
N-[3-(6-chloro-1,3-benzothiazol-2-yl)-1-bicyclo[1.1.1]pentanyl]-3-(methylsulfonylmethyl)-1,2,4-thiadiazole-5-carboxamide;
N-[3-(6-chloro-1,3-benzothiazol-2-yl)-1-bicyclo[1.1.1]pentanyl]-3-(1-methylsulfonylethyl)-1,2,4-thiadiazole-5-carboxamide;
N-[3-(6-chloro-1,3-benzothiazol-2-yl)-1-bicyclo[1.1.1]pentanyl]-3-(1-methyl-1-methylsulfonyl-ethyl)-1,2,4-thiadiazole-5-carboxamide;
N-[3-(6-chloro-1,3-benzothiazol-2-yl)-1-bicyclo[1.1.1]pentanyl]-1-(methylsulfonylmethyl)pyrazole-3-carboxamide;
N-[3-(6-chloro-1,3-benzothiazol-2-yl)-1-bicyclo[1.1.1]pentanyl]-1-(1-methylsulfonylethyl)pyrazole-3-carboxamide;
N-[3-(6-chloro-1,3-benzothiazol-2-yl)-1-bicyclo[1.1.1]pentanyl]-3-(methylsulfonylmethyl)-1,2,4-oxadiazole-5-carboxamide;
N-[3-(6-chloro-1,3-benzothiazol-2-yl)-1-bicyclo[1.1.1]pentanyl]-5-(1-methylsulfonylcyclopropyl)-1,3,4-oxadiazole-2-carboxamide;
N-[3-(6-chloro-1,3-benzothiazol-2-yl)-1-bicyclo[1.1.1]pentanyl]-2-(methylsulfonylmethyl)triazole-4-carboxamide;
N-[3-(6-chloro-1,3-benzothiazol-2-yl)-1-bicyclo[1.1.1]pentanyl]-2-(trifluoromethyl)pyridine-4-carboxamide;
N-(3-(5-chlorobenzo[d]thiazol-2-yl)bicyclo[1.1.1]pentan-1-yl)-5-(methylthio)furan-2-carboxamide;
N-(3-(5-chlorobenzo[d]thiazol-2-yl)bicyclo[1.1.1]pentan-1-yl)-5-(methylsulfonyl)furan-2-carboxamide;
N-(3-(5-chlorobenzo[d]thiazol-2-yl)bicyclo[1.1.1]pentan-1-yl)-5-(cyclopropylsulfonyl)furan-2-carboxamide;
N-(3-(5-chlorobenzo[d]thiazol-2-yl)bicyclo[1.1.1]pentan-1-yl)-5-((methylsulfonyl)methyl)furan-2-carboxamide;
5-(bromo(methylsulfonyl)methyl)-N-(3-(5-chlorobenzo[d]thiazol-2-yl)bicyclo[1.1.1]pentan-1-yl)furan-2-carboxamide;
N-(3-(5-chlorobenzo[d]thiazol-2-yl)bicyclo[1.1.1]pentan-1-yl)-5-(1-(methylsulfonyl)propyl)furan-2-carboxamide;
N-(3-(5-chlorobenzo[d]thiazol-2-yl)bicyclo[1.1.1]pentan-1-yl)-5-(2-(methylsulfonyl)propan-2-yl)furan-2-carboxamide;
N-[3-(5-chloro-1,3-benzothiazol-2-yl)-1-bicyclo[1.1.1]pentanyl]-5-(1-methylsulfonylethyl)furan-2-carboxamide;
N-(3-(5-chlorobenzo[d]thiazol-2-yl)bicyclo[1.1.1]pentan-1-yl)-5-(1-(methylsulfonyl)cyclopropyl)furan-2-carboxamide;
N-(3-(5-chlorobenzo[d]thiazol-2-yl)bicyclo[1.1.1]pentan-1-yl)-5-((2-methoxyethyl)sulfonyl)furan-2-carboxamide;
N-(3-(5-chlorobenzo[d]thiazol-2-yl)bicyclo[1.1.1]pentan-1-yl)-5-((methylthio)methyl)furan-2-carboxamide;
N-(3-(5-chlorobenzo[d]thiazol-2-yl)bicyclo[1.1.1]pentan-1-yl)-5-(2-(methylsulfinyl)propan-2-yl)furan-2-carboxamide;
N-[6-(5-chloro-1,3-benzoxazol-2-yl)spiro[3.3]heptan-2-yl]-1,1-dioxo-thietane-3-carboxamide;
N-[3-(5-chloro-1,3-benzothiazol-2-yl)-1-bicyclo[1.1.1]pentanyl]-2-isopropylsulfonyl-pyridine-4-carboxamide;
2-tert-butylsulfonyl-N-[3-(5-chloro-1,3-benzothiazol-2-yl)-1-bicyclo[1.1.1]pentanyl]pyridine-4-carboxamide;
N-[3-(5-chloro-1,3-benzothiazol-2-yl)-1-bicyclo[1.1.1]pentanyl]-2-(1-methylsulfonylethyl)pyridine-4-carboxamide;
N-[3-(5-chloro-1,3-benzothiazol-2-yl)-1-bicyclo[1.1.1]pentanyl]-2-(1-methyl-1-methylsulfonyl-ethyl)pyridine-4-carboxamide;
N-[3-(6-chloro-1,3-benzothiazol-2-yl)-1-bicyclo[1.1.1]pentanyl]-5-[(1R)-1-methylsulfonylethyl]furan-2-carboxamide;
N-[3-(6-chloro-1,3-benzothiazol-2-yl)-1-bicyclo[1.1.1]pentanyl]-5-[(1S)-1-methylsulfonylethyl]furan-2-carboxamide;
N-[3-(5-chloro-1,3-benzothiazol-2-yl)-1-bicyclo[1.1.1]pentanyl]-5-[(1R)-1-methylsulfonylethyl]furan-2-carboxamide;
N-[3-(6-chloro-1,3-benzothiazol-2-yl)-1-bicyclo[1.1.1]pentanyl]-5-[(1S)-1-methylsulfonylethyl]furan-2-carboxamide;
N-[3-(6-chloro-1,3-benzothiazol-2-yl)-1-bicyclo[1.1.1]pentanyl]-3-(1-methylsulfonylethyl)pyrazole-1-carboxamide;
N-[3-(6-chloro-1,3-benzothiazol-2-yl)-1-bicyclo[1.1.1]pentanyl]-3-(methylsulfonylmethyl)pyrazole-1-carboxamide;
N-[3-(6-chloro-1,3-benzothiazol-2-yl)-1-bicyclo[1.1.1]pentanyl]-3-(1-methyl-1-methylsulfonyl-ethyl)pyrazole-1-carboxamide;
N-[3-(5-bromo-1,3-benzothiazol-2-yl)-1-bicyclo[1.1.1]pentanyl]-3-(1-methyl-1-methylsulfonyl-ethyl)pyrazole-1-carboxamide;
N-[3-(5-bromo-1,3-benzothiazol-2-yl)-1-bicyclo[1.1.1]pentanyl]-3-(1-methylsulfonylcyclopropyl)pyrazole-1-carboxamide;
N-[3-(6-chloro-1,3-benzothiazol-2-yl)-1-bicyclo[1.1.1]pentanyl]-3-(1-methylsulfonylcyclopropyl)pyrazole-1-carboxamide;
N-(3-(5-chlorobenzo[d]thiazol-2-yl)bicyclo[1.1.1]pentan-1-yl)-5-(S-methylsulfonimidoyl)furan-2-carboxamide;
N-[3-(6-chloro-1,3-benzothiazol-2-yl)-1-bicyclo[1.1.1]pentanyl]-5-(cyclopropylsulfonimidoyl)furan-2-carboxamide;
N-[3-(6-chloro-1,3-benzothiazol-2-yl)-1-bicyclo[1.1.1]pentanyl]-5-(cyclopropylmethylsulfonimidoyl)furan-2-carboxamide;
N-[3-(6-chloro-1,3-benzothiazol-2-yl)-1-bicyclo[1.1.1]pentanyl]-2-(1-methylsulfonylcyclopropyl)oxazole-5-carboxamide;
N-[3-(6-chloro-1,3-benzothiazol-2-yl)-1-bicyclo[1.1.1]pentanyl]-5-(1-methylsulfonylcyclopropyl)thiazole-2-carboxamide;
N-[3-(6-chloro-1,3-benzothiazol-2-yl)-1-bicyclo[1.1.1]pentanyl]-5-(1-methylsulfonylcyclopropyl)oxazole-2-carboxamide;
N-[3-(6-chloro-1,3-benzothiazol-2-yl)-1-bicyclo[1.1.1]pentanyl]-5-(cyclopropylmethylsulfonyl)furan-2-carboxamide;
N-[3-(6-chloro-1,3-benzothiazol-2-yl)-1-bicyclo[1.1.1]pentanyl]-5-(cyclopropylmethylsulfinyl)furan-2-carboxamide;
N-[3-(6-chloro-1,3-benzothiazol-2-yl)-1-bicyclo[1.1.1]pentanyl]-5-methylsulfinyl-furan-2-carboxamide;
N-[3-(6-chloro-1,3-benzothiazol-2-yl)-1-bicyclo[1.1.1]pentanyl]-5-cyclopropylsulfinyl-furan-2-carboxamide;
N-(3-(5-chlorobenzo[d]thiazol-2-yl)bicyclo[1.1.1]pentan-1-yl)-5-(dichloro(methylsulfonyl)methyl)furan-2-carboxamide;
5-(chloro(methylsulfonyl)methyl)-N-(3-(5-chlorobenzo[d]thiazol-2-yl)bicyclo[1.1.1]pentan-1-yl)furan-2-carboxamide;
N-[3-(6-chloro-1,3-benzothiazol-2-yl)-1-bicyclo[1.1.1]pentanyl]-3-(1-methylsulfonylcyclopropyl)-1,2,4-oxadiazole-5-carboxamide;
N-[3-(6-bromo-1,3-benzothiazol-2-yl)-1-bicyclo[1.1.1]pentanyl]-5-(1-methylsulfonylcyclopropyl)-1,2,4-oxadiazole-3-carboxamide;
N-[3-(5-bromo-1,3-benzothiazol-2-yl)-1-bicyclo[1.1.1]pentanyl]-5-(1-methylsulfonylcyclopropyl)-1,2,4-oxadiazole-3-carboxamide;
5-(1-methylsulfonylcyclopropyl)-N-[3-(6-morpholino-1,3-benzothiazol-2-yl)-1-bicyclo[1.1.1]pentanyl]furan-2-carboxamide;
5-(1-methylsulfonylcyclopropyl)-N-[3-(5-morpholino-1,3-benzothiazol-2-yl)-1-bicyclo[1.1.1]pentanyl]furan-2-carboxamide;
N-[3-[6-(2-methoxyethoxy)-1,3-benzothiazol-2-yl]-1-bicyclo[1.1.1]pentanyl]-5-(1-methylsulfonylcyclopropyl)furan-2-carboxamide;
N-[3-(6-chloro-1,3-benzothiazol-2-yl)-1-bicyclo[1.1.1]pentanyl]-3-(1-methylsulfonylethyl)-1H-pyrazole-5-carboxamide;
5-(butanoylsulfamoyl)-N-[3-(6-chloro-1,3-benzothiazol-2-yl)-1-bicyclo[1.1.1]pentanyl]furan-2-carboxamide;

or a pharmaceutically acceptable salt thereof.

### Pharmaceutical Compositions and Administration

The invention also relates to a compound of formula (I) or a pharmaceutically acceptable salt thereof for use as therapeutically active substance. Another embodiment provides pharmaceutical compositions or medicaments containing the compounds of the invention and a therapeutically inert carrier, diluent or excipient, as well as methods of using the compounds of the invention to prepare such compositions and medicaments. In one example, compounds of formula (I) may be formulated by mixing at ambient temperature at the appropriate pH, and at the desired degree of purity, with physiologically acceptable carriers, i.e., carriers that are non-toxic to recipients at the dosages and concentrations employed into a galenical administration form. The pH of the formulation depends mainly on the particular use and the concentration of compound, but preferably ranges anywhere from about 3 to about 8. In one example, a compound of formula (I) is formulated in an acetate buffer, at pH 5. In another embodiment, the compounds of formula (I) are sterile. The compound may be stored, for example, as a solid or amorphous composition, as a lyophilized formulation or as an aqueous solution.

Compositions are formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The “effective amount” of the compound to be administered will be governed by such considerations, and is the minimum amount necessary to reduction of HBsAg and HBeAg in HBV patients. For example, such amount may be below the amount that is toxic to normal cells, or the mammal as a whole.

In one example, the pharmaceutically effective amount of the compound of the invention administered parenterally per dose will be in the range of about 0.1 to 100 mg/kg, alternatively about 0.1 to 50 mg/kg of patient body weight per day, with the typical initial range of compound used being 0.3 to 15 mg/kg/day. In another embodiment, oral unit dosage forms, such as tablets and capsules, preferably contain from about 25 to about 1000 mg of the compound of the invention.

The compounds of the invention may be administered by any suitable means, including oral, topical (including buccal and sublingual), rectal, vaginal, transdermal, parenteral, subcutaneous, intraperitoneal, intrapulmonary, intradermal, intrathecal and epidural and intranasal, and, if desired for local treatment, intralesional administration. Parenteral infusions include intramuscular, intravenous, intraarterial, intraperitoneal, or subcutaneous administration.

The compounds of the present invention may be administered in any convenient administrative form, e.g., tablets, powders, capsules, solutions, dispersions, suspensions, syrups, sprays, suppositories, gels, emulsions, patches, etc. Such compositions may contain components conventional in pharmaceutical preparations, e.g., diluents, carriers, pH modifiers, sweeteners, bulking agents, and further active agents.

A typical formulation is prepared by mixing a compound of the present invention and a carrier or excipient. Suitable carriers and excipients are well known to those skilled in the art and are described in detail in, e.g., Ansel, Howard C., et al., Ansel's Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams & Wilkins, 2000; and Rowe, Raymond C. Handbook of Pharmaceutical Excipients. Chicago, Pharmaceutical Press, 2005. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, diluents and other known additives to provide an elegant presentation of the drug (i.e., a compound of the present invention or pharmaceutical composition thereof) or aid in the manufacturing of the pharmaceutical product (i.e., medicament).

An example of a suitable oral dosage form is a tablet containing about 25 to 500 mg of the compound of the invention compounded with about 90 to 30 mg anhydrous lactose, about 5 to 40 mg sodium croscarmellose, about 5 to 30 mg polyvinylpyrrolidone (PVP) K30, and about 1 to 10 mg magnesium stearate. The powdered ingredients are first mixed together and then mixed with a solution of the PVP. The resulting composition can be dried, granulated, mixed with the magnesium stearate and compressed to tablet form using conventional equipment. An example of an aerosol formulation can be prepared by dissolving the compound, for example 5 to 400 mg, of the invention in a suitable buffer solution, e.g. a phosphate buffer, adding a tonicifier, e.g. a salt such sodium chloride, if desired. The solution may be filtered, e.g., using a 0.2 micron filter, to remove impurities and contaminants.

An embodiment, therefore, includes a pharmaceutical composition comprising a compound of formula (I), or a pharmaceutically acceptable salt thereof.

In a further embodiment includes a pharmaceutical composition comprising a compound of formula (I), or a pharmaceutically acceptable salt thereof, together with a pharmaceutically acceptable carrier or excipient.

Another embodiment includes a pharmaceutical composition comprising a compound of formula (I), or a pharmaceutically acceptable salt thereof for use in the treatment of HBV infection.

The following embodiments illustrate typical compositions of the present invention, but serve merely as representative thereof.

Composition A

A compound of the present invention can be used in a manner known per se as the active ingredient for the production of tablets of the following composition:

| | Per tablet |
|---|---|
| Active ingredient | 200 mg |
| Microcrystalline cellulose | 155 mg |
| Corn starch | 25 mg |
| Talc | 25 mg |
| Hydroxypropylmethylcellulose | 20 mg |
| | 425 mg |

Composition B

A compound of the present invention can be used in a manner known per se as the active ingredient for the production of capsules of the following composition:

| | Per capsule |
|---|---|
| Active ingredient | 100.0 mg |
| Corn starch | 20.0 mg |
| Lactose | 95.0 mg |
| Talc | 4.5 mg |
| Magnesium stearate | 0.5 mg |
| | 220.0 mg |

### Indications and Methods of Treatment

The compounds of the invention have anti-HBV activity. Accordingly, the compounds of the invention are useful for the treatment or prophylaxis of HBV infection.

The invention also relates to the use of a compound of formula (I) for the inhibition of HBeAg.

The invention further relates to the use of a compound of formula (I) for the inhibition of HBsAg.

The invention relates to the use of a compound of formula (I) for the inhibition of HBV DNA.

The invention relates to the use of a compound of formula (I) for use in the treatment or prophylaxis of HBV infection.

The use of a compound of formula (I) for the preparation of medicaments useful in the treatment or prophylaxis diseases that are related to HBV infection is an object of the invention.

The invention relates in particular to the use of a compound of formula (I) for the preparation of a medicament for the treatment or prophylaxis of HBV infection.

Another embodiment includes a method for the treatment or prophylaxis of HBV infection, which method comprises administering an effective amount of a compound of formula (I), or a pharmaceutically acceptable salt thereof.

The invention relates in particular to a compound of formula (I) for use in the treatment or prophylaxis of HBV infection.

### Synthesis

The compounds of the present invention can be prepared by any conventional means. Suitable processes for synthesizing these compounds as well as their starting materials are provided in the schemes below and in the examples. All substituents, in particular, R¹, R², L and A are as defined above unless otherwise indicated. Furthermore, and unless explicitly otherwise stated, all reactions, reaction conditions, abbreviations and symbols have the meanings well known to a person of ordinary skill in organic chemistry.

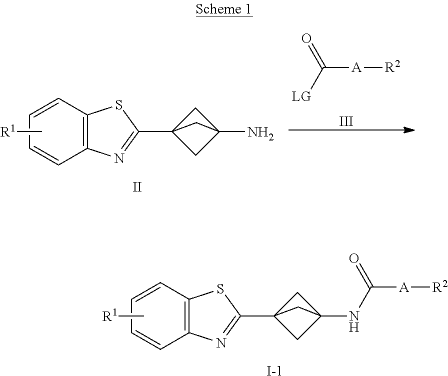

wherein LG is OH or C₁₋₆alkoxy.

Compound of formula II (which can be synthesized from 6-bromo-2-hydroxybenzothiazole (CAS No: 62266-82-4)) reacts with compound of formula III (which are synthesized from commercially available starting materials) in the presence of a coupling reagent, such as HATU, T₃P or AlMe₃, and a base such as TEA or DIPEA or no base, in a solvent such as DMF, DCM or toluene, to afford compound of formula I-1.

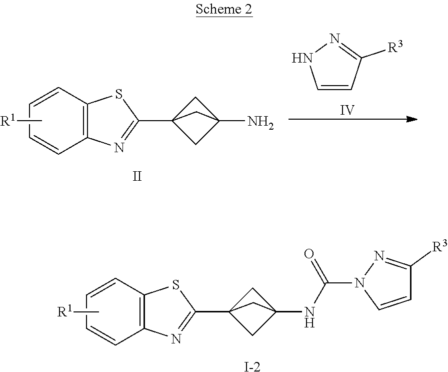

wherein R³ is C₁₋₆alkylsulfonylC₁₋₆alkyl or C₁₋₆alkylsulfonylC₃₋₇cycloalkyl.

Compound of formula II is treated with triphosgene in the presence of a base such as TEA or DIPEA, in a suitable solvent such as THF or DCM, then reacts with compound of formula IV (which was synthesized from 1H-pyrazole-3-carboxylate (CAS No: 15366-34-4)), to afford compound of formula I-2.

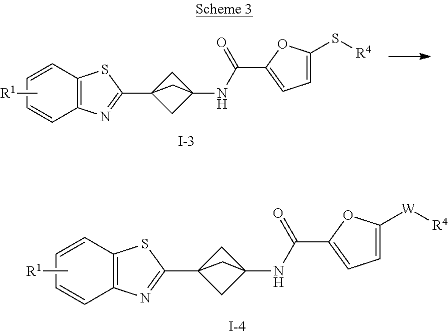

wherein R⁴ is C₁₋₆alkyl, C₃₋₇cycloalkyl, C₃₋₇cycloalkylC₁₋₆alkyl, C₁₋₆alkoxyC₁₋₆alkyl, or C₁₋₆alkylcarbonyl; W is S(O), S(O)₂ or S(O)(NH).

Oxidation of compound of formula I-3 in the presence of an oxidate, such as m-CPBA, or PhI(OAc)₂ and (NH₄)₂CO₃, in a suitable solvent, such as MeOH or DCM, affords compound of formula I-4.

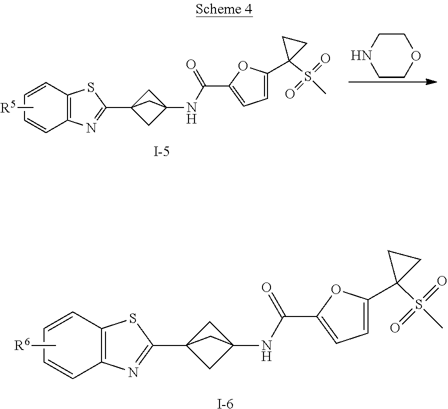

wherein R⁵ is halogen; R⁶ is morpholinyl.

Compound of formula I-5 reacts with morpholine in the presence of X-phos G3, and a base such as Cs₂CO₃, in a suitable solvent such as dioxane, affords compound of formula I-6.

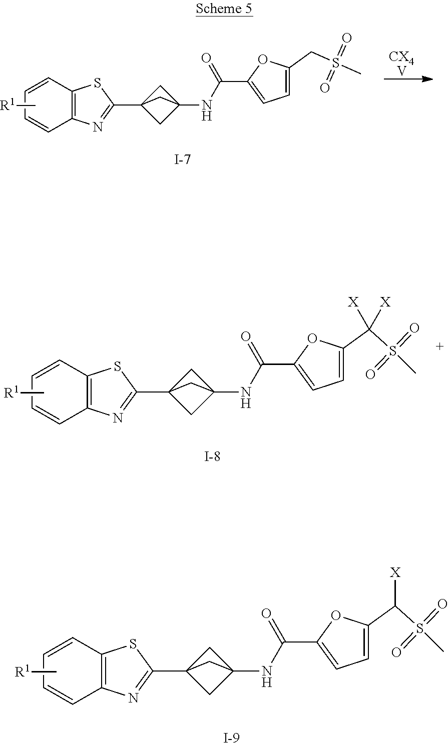

wherein X is halogen.

Compound of formula I-7 reacts with compound of formula CX₄ in the presence of a base as NaOH or KOH, in a suitable solvent such as DMF, affords compound of formula I-8 and compound of formula I-9.

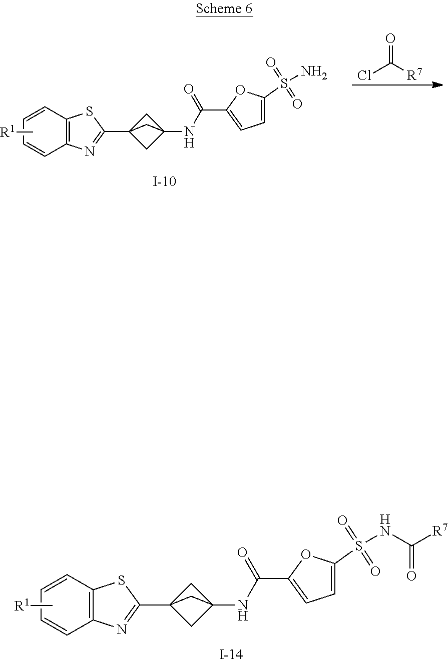

wherein R⁷ is C₁₋₆alkyl.

Compound of formula I-10 reacts with acyl chloride VI in the presence of a base, such as TEA, DIPEA or K₂CO₃, in a solvent such as DCM or DMF, to afford compound of formula I-11.

### EXAMPLES

The invention will be more fully understood by reference to the following examples. They should not, however, be construed as limiting the scope of the invention.

Abbreviations used herein are as follows:
ACN Acetonitrile
AIBN Azodiisobutyronitrile
BPO Dibenzoyl peroxide
CDCl₃ Deuterated chloroform
CD₃OD Deuterated methanol
DCM Dichloromethane
DIEA N,N-diisopropylethylamine
DIPEA N-ethyldiisopropylamine
DMAP 4-Dimethylaminopyridine
DMF Dimethylformamide
DMFDMA N,N-dimethylformamide dimethyl acetal
DMSO-d₆ Deuterated dimethylsulfoxide
EDCI 1-Ethyl-3-(3-dimethylaminopropyl)carbodiimide hydrochloride
EtOAc Ethyl acetate
HATU O-(7-aza-1H-benzotriazole-1-yl)-N,N,N′,N′-tetramethyluronium hexafluorophosphate
HPLC High performance liquid chromatography
h Hour
IC₅₀ The half maximal inhibitory concentration
LC-MS Liquid chromatography-mass spectrometry
m-CPBA 3-chloroperbenzoic acid
MeOH Methanol
M Molarity
MHz Megahertz
min Minute
mL Milliliter
mmol Millimole
MS (ESI) Mass spectroscopy (electron spray ionization)
NBS N-bromosuccinimide
obsd. Observed
OMe Methoxy
OEt Ethyoxy
PIDA (Diacetoxyiodo)benzene
PPA Polyphosphoric acid
Pd(dppf)Cl₂ Dichloro(1,1′-bis(diphenylphosphanyl)ferrocene)palladium(II) complex
Pd₂(dba)₃ Tris(dibenzylideneacetone)dipalladium(0)
PhI(OAc)₂ Iodobenzene diacetate
SEMCl 2-(Trimethylsilyl)ethoxymethyl chloride
SFC Supercritical fluid chromatography
TEA Triethylamine
TFA Trifluoroacetic acid
THF Tetrahydrofuran
TMSCN Trimethylsilyl cyanide
T₃P 2,4,6-Tripropyl-1,3,5,2,4,6-trioxatriphosphinane 2,4,6-trioxide
TsCl 4-Toluenesulfonyl chloride
Xantphos 9,9-Dimethyl-4,5-bis(diphenylphosphino)xanthene
X-phos G3 Methanesulfonato(2-dicyclohexylphosphino-2′,4′,6′-tri-i-propyl-1,1′-biphenyl)(2′-amino-1,1′-biphenyl-2-yl)palladium(II)
δ chemical shift

### GENERAL EXPERIMENTAL CONDITIONS

Intermediates and final compounds were purified by flash chromatography using one of the following instruments: i) Biotage SP1 system and the Quad 12/25 Cartridge module. ii) ISCO combi-flash chromatography instrument. Silica gel Brand and pore size: i) KP-SIL 60 Å, particle 5 size: 40-60 μm; ii) CAS registry NO: Silica Gel: 63231-67-4, particle size: 47-60 micron silica gel; iii) ZCX from Qingdao Haiyang Chemical Co., Ltd, pore: 200-300 or 300-400.

Intermediates and final compounds were purified by preparative HPLC on reversed phase column using X Bridge™ Perp Cis (5 μm, OBD™ 30×100 mm) column or SunFire™ Perp C₁₈ (5 μm, OBD™ 30×100 mm) column.

LC-MS spectra were obtained using an Acquity Ultra Performance LC—3100 Mass Detector or Acquity Ultra Performance LC—SQ Detector. Standard LC-MS conditions were as follows (running time 3 minutes):

Acidic condition: A: 0.1% formic acid in H₂O; B: 0.1% formic acid in acetonitrile;

Basic condition: A: 0.05% NH₃·H₂O in H₂O; B: acetonitrile;

Neutral condition: A: H₂O; B: acetonitrile.

Mass spectra (MS): generally only ions which indicate the parent mass are reported, and unless otherwise stated the mass ion quoted is the positive mass ion (M+H)+.

The microwave assisted reactions were carried out in a Biotage Initiator Sixty or CEM Discover.

NMR Spectra were obtained using Bruker Avance 400 MHz.

All reactions involving air-sensitive reagents were performed under an argon atmosphere. Reagents were used as received from commercial suppliers without further purification unless otherwise noted.

### PREPARATIVE EXAMPLES

The invention will be more fully understood by reference to the following examples. They should not, however, be construed as limiting the scope of the invention.

### Intermediate Int-1

### 3-(6-bromo-1,3-benzothiazol-2-yl)bicyclo[1.1.1]pentan-1-amine

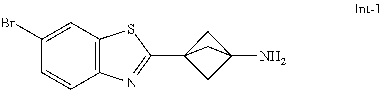 [SMILES: NC12CC(c3nc4ccc(Br)cc4s3)(C1)C2]

The title compound was prepared according to the following scheme:

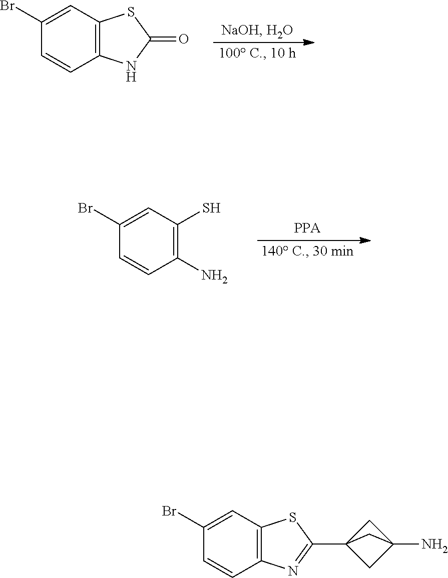

### Step 1: Preparation of 2-amino-5-bromo-benzenethiol

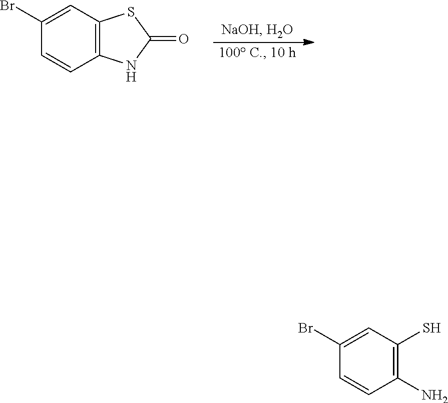

To a solution of NaOH (0.45 g, 434 mmol) in water (200 mL) was added 6-bromo-2-benzothiazolinone (20.0 g, 87 mmol, CAS No: 62266-82-4). Then the reaction was stirred at 100° C. for 10 h. The reaction mixture was acidified by aqueous HCl (1 M) to pH=3. The precipitate was filtrated and washed with water to give the crude product 2-amino-5-bromo-benzenethiol (15.0 g) as a yellow solid.

MS obsd. (ESI⁺) [(M+H)⁺]: 204.0.

### Step 2: Preparation of 3-(6-bromo-1,3-benzothiazol-2-yl)bicyclo[1.1.1]pentan-1-amine (Int-1)

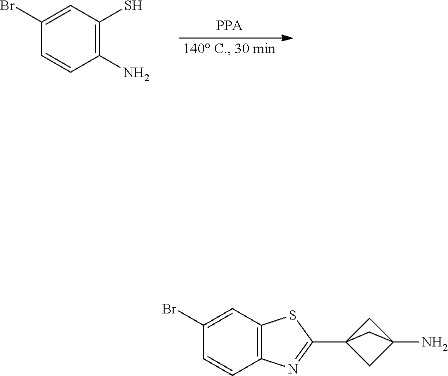 [SMILES: Nc1ccc(Br)cc1S ; "PPA" ; "140° C., 30 min" ; NC12CC(c3nc4ccc(Br)cc4s3)(C1)C2]

A mixture of 2-amino-5-bromo-benzenethiol (1.0 g, 4.9 mmol) and 3-(tert-butoxycarbonylamino) bicyclo [1.1.1] pentane-2-carboxylic acid (1.23 g, 5.4 mmol, CAS No: 303752-38-7) in PPA (5 mL) was stirred at 140° C. for 30 min. After cooling to 0° C., the reaction was quenched with aqueous ammonium hydroxide solution (4 M). The resulting solution was diluted with water (30 mL) and extracted with EtOAc (20 mL×3). The organic layers were concentrated in vacuum to give 3-(6-bromo-1,3-benzothiazol-2-yl)bicyclo[1.1.1]pentan-1-amine (1.45 g) as a yellow solid, which was used in next step without further purification.

MS obsd. (ESI⁺) [(M+H)⁺]: 295.2.

### Intermediate Int-2

### 3-(5-bromo-1,3-benzothiazol-2-yl)bicyclo[1.1.1]pentan-1-amine

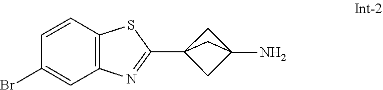 [SMILES: NC12CC(c3nc4cc(Br)ccc4s3)(C1)C2]

The title compound was prepared in analogy to the procedure described for the preparation of 3-(6-bromo-1,3-benzothiazol-2-yl)bicyclo[1.1.1]pentan-1-amine (Int-1), by using 5-bromo-2-benzothiazolinone instead of 6-bromo-2-benzothiazolinone.

### Intermediate Int-3

### 2-(3-amino-1-bicyclo[1.1.1]pentanyl)-1,3-benzothiazole-6-carbonitrile

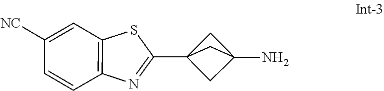 [SMILES: N#Cc1ccc2nc(C34CC(N)(C3)C4)sc2c1]

The title compound was prepared according to the following scheme:

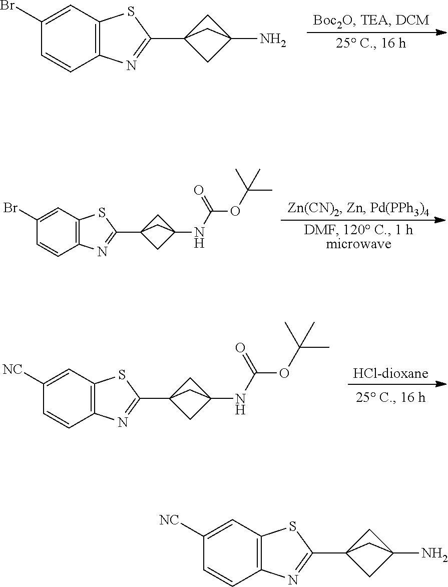

### Step 1: Preparation of tert-butyl N-[1-(6-bromo-1,3-benzothiazol-2-yl)-3-bicyclo [1.1.1]pentanyl] carbamate

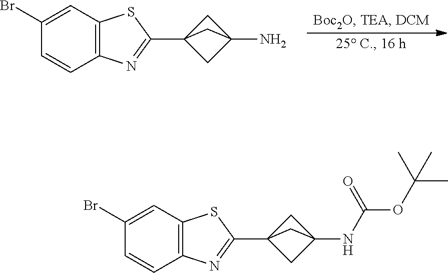 [SMILES: NC12CC(c3nc4ccc(Br)cc4s3)(C1)C2 ; "25° C., 16 h" ; "Boc2O, TEA, DCM" ; CC(C)(C)OC(=O)NC12CC(c3nc4ccc(Br)cc4s3)(C1)C2]

To a solution of 3-(6-bromo-1,3-benzothiazol-2-yl)-1-methyl-bicyclo [1.1.1] pentan-2-amine (1.45 g, 4.68 mmol) in DCM (10 mL) was added Boc₂O (1.00 g, 4.68 mmol) and triethylamine (1.00 g, 9.37 mmol). The mixture was stirred at 25° C. for 16 h and then concentrated in vacuum. The residue was diluted with EtOAc (50 mL) and washed with water (30 mL×2) and brine (30 mL). The organic layers were dried over Na₂SO₄ and concentrated in vacuum to give tert-butyl N-[1-(6-bromo-1,3-benzothiazol-2-yl)-3-bicyclo [1.1.1] pentanyl]carbamate (1.12 g) as a yellow solid.

MS obsd. (ESI⁺) [(M+H)⁺]: 395.2.

### Step 2: Preparation of tert-butyl N-[1-(6-cyano-1,3-benzothiazol-2-yl)-3-bicyclo[1.1.1]pentanyl]carbamate

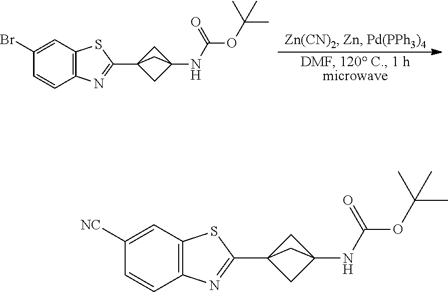

To a suspension of [3-(6-bromo-1,3-benzothiazol-2-yl)-1-bicyclo[1.1.1]pentanyl]methanamine (1.0 g, 2.53 mmol), zinc cyanide (0.59 g, 5.06 mmol), zinc powder (0.016 g, 0.25 mmol) in DMF (5 mL) was added Pd(PPh₃)₄ (0.29 g, 0.253 mmol) at 25° C. under nitrogen atmosphere. Then the reaction was stirred at 120° C. for 1 h under microwave. After cooled to ambient temperature, the resulted solution was diluted with EtOAc (60 mL) and washed with water (15 mL×2) and brine (15 mL). The organic layer was dried over Na₂SO₄ and concentrated in vacuum. The residue was purified by silica gel column eluted with EtOAc/PE=30/70 to give tert-butyl N-[1-(6-cyano-1,3-benzothiazol-2-yl)-3-bicyclo[1.1.1]pentanyl]carbamate (0.32 g) as a light yellow solid.

MS obsd. (ESI⁺) [(M+H)⁺]: 342.1.

### Step 3: Preparation of 2-(3-amino-1-bicyclo[1.1.1]pentanyl)-1,3-benzothiazole-6-carbonitrile (Int-3)

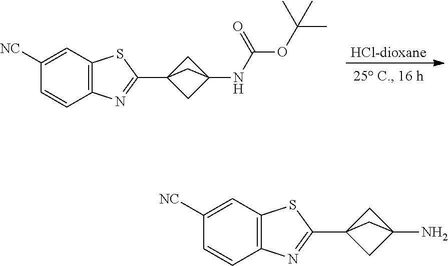 [SMILES: CC(C)(C)OC(=O)NC12CC(c3nc4ccc(C#N)cc4s3)(C1)C2 ; "25° C., 16 h" ; "HCl-dioxane" ; N#Cc1ccc2nc(C34CC(N)(C3)C4)sc2c1]

To a solution of HCl in dioxane (2 mL, 1 M) was added tert-butyl N-[1-(6-cyano-1,3-benzothiazol-2-yl)-3-bicyclo[1.1.1]pentanyl]carbamate (323 mg, 0.95 mmol). The mixture was stirred at 25° C. for 16 h. The resulting solution was concentrated in vacuum to give 2-(3-amino-1-bicyclo[1.1.1]pentanyl)-1,3-benzothiazole-6-carbonitrile (135 mg) as a light yellow solid.

MS obsd. (ESI⁺) [(M+H)⁺]: 242.1

### Intermediate Int-4

### 2-(3-amino-1-bicyclo[1.1.1]pentanyl)-1,3-benzothiazole-5-carbonitrile

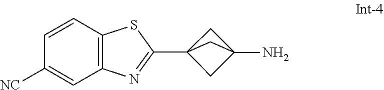 [SMILES: N#Cc1ccc2sc(C34CC(N)(C3)C4)nc2c1]

The title compound was prepared according to the following scheme:

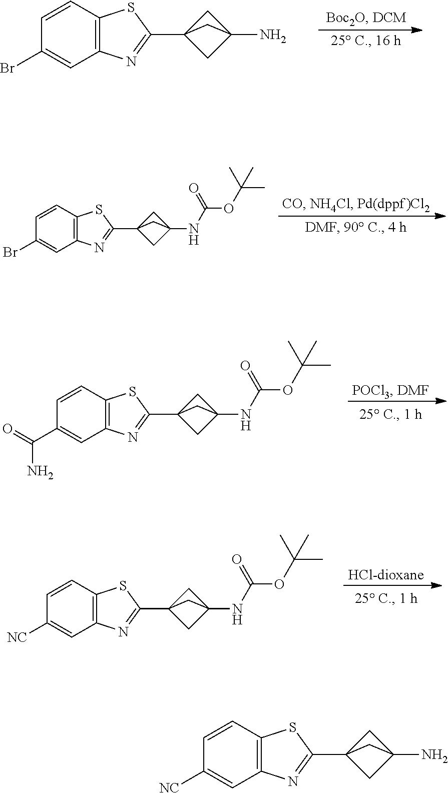

### Step 1: Preparation of tert-butyl N-[1-(5-bromo-1,3-benzothiazol-2-yl)-3-bicyclo[1.1.1]pentanyl]carbamate

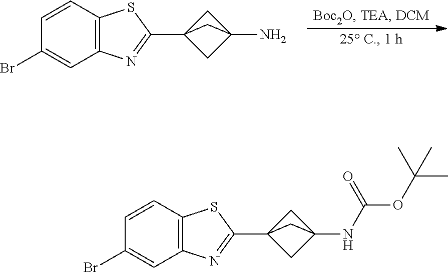 [SMILES: NC12CC(c3nc4cc(Br)ccc4s3)(C1)C2 ; "25° C., 1 h" ; "Boc2O, TEA, DCM" ; CC(C)(C)OC(=O)NC12CC(c3nc4cc(Br)ccc4s3)(C1)C2]

To a solution of 1-(5-bromo-1,3-benzothiazol-2-yl)bicyclo[1.1.1]pentan-3-amine (Int-2) (1.0 g, 3.39 mmol) in DCM (10 mL) was added Boc₂O (0.92 g, 4.07 mmol). The reaction was stirred at 25° C. for 1 h. The resulting solution was diluted with EtOAc (200 mL) and washed with water (100 mL×2). The organic layer was dried over Na₂SO₄ and concentrated in vacuum. The residue was purified by silica gel column eluted with PE/EtOAc=9/1 to give tert-butyl N-[1-(5-bromo-1,3-benzothiazol-2-yl)-3-bicyclo[1.1.1]pentanyl]carbamate (0.88 g) as a brown solid.

MS obsd. (ESI⁺) [(M+H)⁺]: 395.0.

### Step 2: Preparation of tert-butyl N-[1-(5-carbamoyl-1,3-benzothiazol-2-yl)-3-bicyclo[1.1.1]pentanyl]carbamate

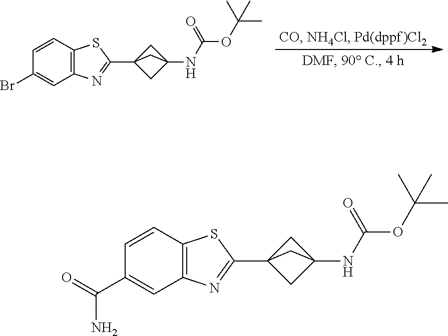 [SMILES: CC(C)(C)OC(=O)NC12CC(c3nc4cc(Br)ccc4s3)(C1)C2 ; "CO, NH4Cl, Pd(dppf)Cl2" ; "DMF, 90° C., 4 h" ; CC(C)(C)OC(=O)NC12CC(c3nc4cc(C(N)=O)ccc4s3)(C1)C2]

To a solution of tert-butyl N-[1-(5-bromo-1,3-benzothiazol-2-yl)-3-bicyclo[1.1.1]pentanyl]carbamate (400 mg, 1.01 mmol) in DMF (3 mL) was added ammonium chloride (65 mg, 1.21 mmol) and Pd(dppf)Cl₂ (74 mg, 0.100 mmol) under carbon monoxide atmosphere. The reaction was stirred at 90° C. for 4 h. After cooled to room temperature, the resulted solution was diluted with EtOAc (50 mL) and washed with water (20 mL×2). The organic layer was dried over Na₂SO₄ and concentrated in vacuum. The residue was purified by silica gel column eluted with PE/EtOAc=4/1 to give tert-butyl N-[1-(5-carbamoyl-1,3-benzothiazol-2-yl)-3-bicyclo[1.1.1]pentanyl]carbamate (316 mg) as a light brown solid.

MS obsd. (ESI⁺) [(M+H)⁺]: 360.1.

### Step 3: Preparation of tert-butyl N-[3-(5-cyano-1,3-benzothiazol-2-yl)-1-bicyclo[1.1.1]pentanyl]carbamate

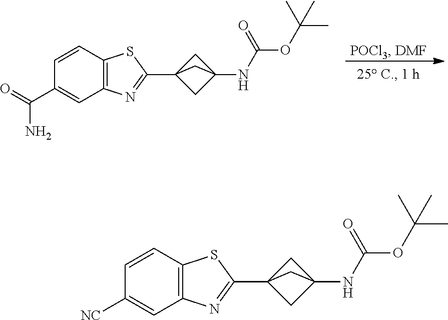

To a solution of tert-butyl N-[1-(5-carbamoyl-1,3-benzothiazol-2-yl)-3-bicyclo[1.1.1]pentanyl]carbamate (120 mg, 0.330 mmol) in DMF (4 mL) was added POCl₃ (51 mg, 0.330 mmol). The reaction was stirred at room temperature for 1 h. The resulting solution was diluted with EtOAc (20 mL) and washed with brine (10 mL×3). The organic layer was dried over Na₂SO₄ and concentrated in vacuum to give tert-butyl N-[1-(5-carbonocyanidoyl-1,3-benzothiazol-2-yl)-3-bicyclo[1.1.1]pentanyl]carbamate (89 mg) as a brown solid.

MS obsd. (ESI⁺) [(M+H)⁺]: 342.0.

### Step 4: Preparation of 2-(3-amino-1-bicyclo[1.1.1]pentanyl)-1,3-benzothiazole-5-carbonitrile (Int-4)

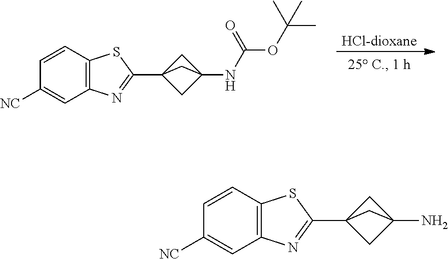 [SMILES: CC(C)(C)OC(=O)NC12CC(c3nc4cc(C#N)ccc4s3)(C1)C2 ; "25° C., 1 h" ; "HCl-dioxane" ; N#Cc1ccc2sc(C34CC(N)(C3)C4)nc2c1]

A mixture of tert-butyl N-[1-(5-carbonocyanidoyl-1,3-benzothiazol-2-yl)-3-bicyclo[1.1.1]pentanyl]carbamate (89 mg, 0.260 mmol) in HCl-dioxane (3 mL, 7M) was stirred at room temperature for 1 h. The mixture was concentrated in vacuum to give 2-(3-amino-1-bicyclo[1.1.1]pentanyl)-1,3-benzothiazole-5-carbonitrile (300 mg) as a brown solid.

MS obsd. (ESI⁺) [(M+H)⁺]: 242.0.

### Intermediate Int-5

### 3-(6-ethynyl-1,3-benzothiazol-2-yl)bicyclo[1.1.1]pentan-1-amine

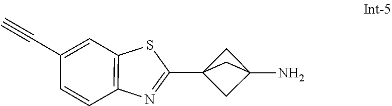 [SMILES: C#Cc1ccc2nc(C34CC(N)(C3)C4)sc2c1]

The title compound was prepared according to the following scheme:

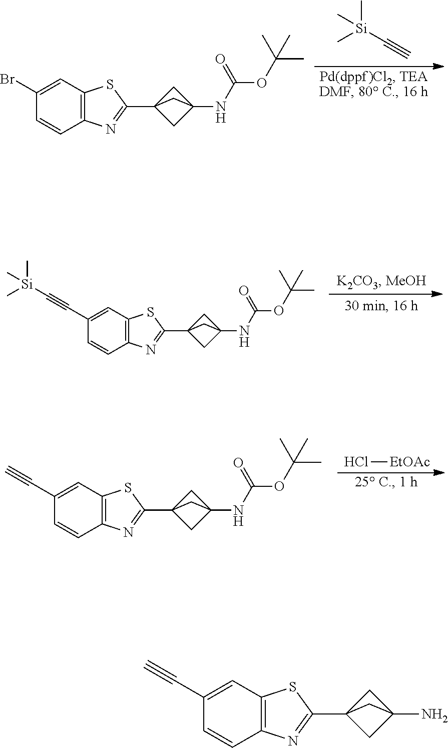

### Step 1: Preparation of tert-butyl N-[1-[6-(2-trimethylsilylethynyl)-1,3-benzothiazol-2-yl]-3-bicyclo [1.1.1] pentanyl] carbamate

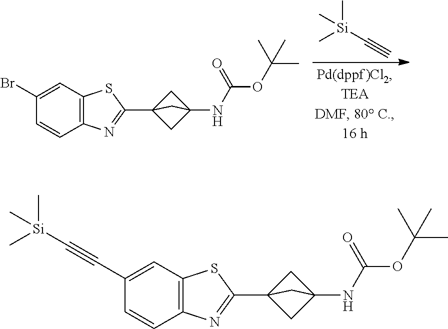

To a solution of [3-(6-bromo-1,3-benzothiazol-2-yl)-1-bicyclo [1.1.1] pentanyl]methanamine (300 mg, 0.761 mmol) in DMF (5 mL) were added trimethylsilylacetylene (86 mg, 0.990 mmol), triethylamine (331 mg, 3.27 mmol) and Pd(dppf)Cl₂ (55 mg, 0.076 mmol) at room temperature. The resulted mixture was stirred at 80° C. for 16 h under nitrogen atmosphere. After cooled to 25° C., the resulting solution was diluted with water (30 mL) and extracted with EtOAc (20 mL×3). The organic layers were dried over Na₂SO₄ and concentrated in vacuum. The residue was purified by silica gel column eluted with PE/EtOAc=1/1 to give tert-butyl N-[1-[6-(2-trimethylsilylethynyl)-1,3-benzothiazol-2-yl]-3-bicyclo [1.1.1] pentanyl] carbamate (130 mg) as a light yellow solid.

MS obsd. (ESI⁺) [(M+H)⁺]: 413.2.

### Step 2: Preparation of tert-butyl N-[1-(6-ethynyl-1,3-benzothiazol-2-yl)-3-bicyclo [1.1.1]pentanyl] carbamate

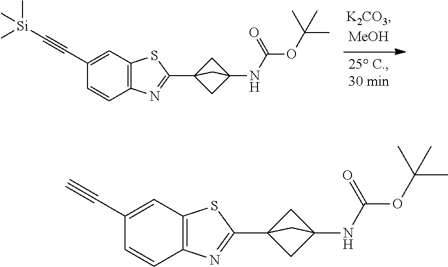

To a solution of tert-butyl N-[1-[6-(2-trimethylsilylethynyl)-1,3-benzothiazol-2-yl]-3-bicyclo [1.1.1] pentanyl] carbamate (113 mg, 0.315 mmol) in methanol (5 mL) was added potassium carbonate (87 mg, 0.630 mmol) at room temperature. The reaction mixture was stirred at 25° C. for 30 min. After removing the solvent, the residue was diluted with water (30 mL) and extracted with EtOAc (20 mL×3). The organic layers were concentrated in vacuum to give tert-butyl N-[1-(6-ethynyl-1,3-benzothiazol-2-yl)-3-bicyclo[1.1.1]pentanyl] (86 mg) as a light yellow solid.

MS obsd. (ESI⁺) [(M+H)⁺]: 341.1.

### Step 3: Preparation of 1-(6-ethynyl-1,3-benzothiazol-2-yl)bicyclo[1.1.1]pentan-3-amine (Int-5)

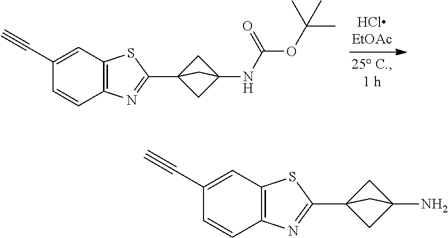

A solution of tert-butyl N-[1-(6-cyano-1,3-benzothiazol-2-yl)-3-bicyclo [1.1.1] pentanyl]carbamate (100 mg, 0.294 mmol) in HCl-EtOAc (4M, 10 mL) was stirred at 25° C. for 30 min. The resulting solution was concentrated in vacuum to give 3-(6-ethynyl-1,3-benzothiazol-2-yl)bicyclo[1.1.1]pentan-1-amine (43 mg) as a light yellow solid.

MS obsd. (ESI⁺) [(M+H)⁺]: 241.1.

### Intermediate Int-6

### 3-(6-fluoro-1,3-benzothiazol-2-yl)bicyclo[1.1.1]pentan-1-amine

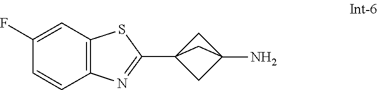 [SMILES: NC12CC(c3nc4ccc(F)cc4s3)(C1)C2]

The title compound was prepared in analogy to the procedure described for the preparation of 3-(6-bromo-1,3-benzothiazol-2-yl)bicyclo[1.1.1]pentan-1-amine (Int-1), by using 2-amino-5-fluoro-benzenethiol instead of 2-amino-5-bromo-benzenethiol.

MS obsd. (ESI⁺) [(M+H)⁺]: 235.1

### Intermediate Int-7

### 3-(6-chloro-1,3-benzothiazol-2-yl)bicyclo[1.1.1]pentan-1-amine

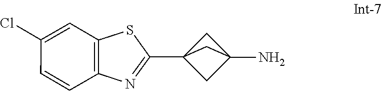 [SMILES: NC12CC(c3nc4ccc(Cl)cc4s3)(C1)C2]

The title compound was prepared in analogy to the procedure described for the preparation of 3-(6-bromo-1,3-benzothiazol-2-yl)bicyclo[1.1.1]pentan-1-amine (Int-1), by using 2-amino-6-chlorobenzothiazole instead of 2-amino-5-bromo-benzenethiol.

### Intermediate Int-8

### 3-(5-chloro-1,3-benzothiazol-2-yl)bicyclo[1.1.1]pentan-1-amine

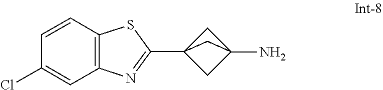 [SMILES: NC12CC(c3nc4cc(Cl)ccc4s3)(C1)C2]

The title compound was prepared in analogy to the procedure described for the preparation of 3-(6-bromo-1,3-benzothiazol-2-yl)bicyclo[1.1.1]pentan-1-amine (Int-1), by using 2-amino-4-chloro-benzenethiol instead of 2-amino-5-bromo-benzenethiol.

### Intermediate Int-9

### 5-(1-methylsulfonylcyclopropyl)furan-2-carboxylic Acid

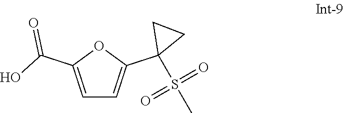

The title compound was prepared according to the following scheme:

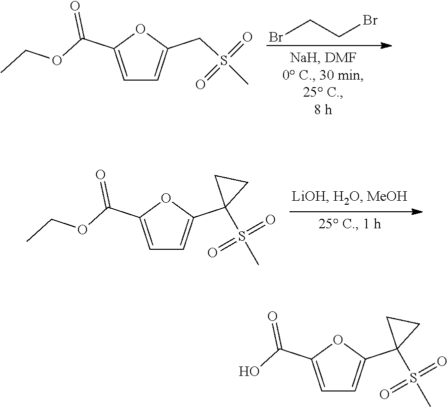

### Step 1: Preparation of ethyl 5-(1-methylsulfonylcyclopropyl)furan-2-carboxylate

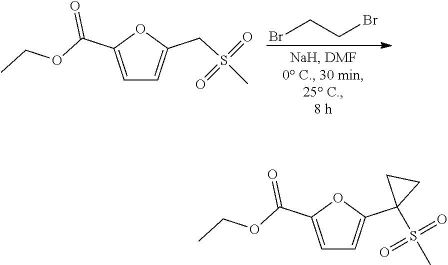

To a solution of ethyl 5-(methylsulfonylmethyl)furan-2-carboxylate (500 mg, 2.15 mmol) in anhydrous DMF (15 mL) was added sodium hydride (155 mg, 6.46 mmol, 60% in oil) at 0° C. After stirring at 0° C. for 30 min, 1,2-dibromoethane (0.19 mL, 2.15 mmol) was added. The reaction was stirred at 25° C. for 8 h and then quenched with water (1 mL). The resulting solution was diluted with EtOAc (100 mL) and washed with water (50 mL×4) and brine (100 mL). The organic layer was dried over Na₂SO₄ and concentrated in vacuum. The residue was purified by silica gel column eluted with PE/EtOAc=1/1 to give ethyl 5-(1-methylsulfonylcyclopropyl)furan-2-carboxylate (200 mg) as light yellow oil.

MS obsd. (ESI⁺) [(M+H)⁺]: 259.0.

### Step 2: Preparation of 5-(1-methylsulfonylcyclopropyl)furan-2-carboxylic Acid (Int-9)

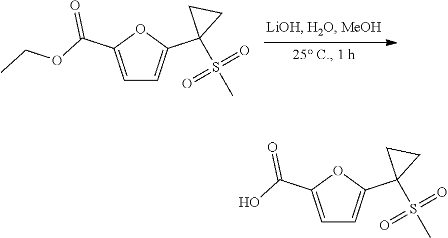 [SMILES: CCOC(=O)c1ccc(C2(S(C)(=O)=O)CC2)o1 ; CS(=O)(=O)C1(c2ccc(C(=O)O)o2)CC1]

To a solution of ethyl 5-(1-methylsulfonylcyclopropyl)furan-2-carboxylate (100 mg, 0.390 mmol) in methanol (5 mL) was added LiOH·H₂O (17 mg, 0.430 mmol). The reaction mixture was stirred at 25° C. for 1 h and then concentrated in vacuum to give 5-(1-methylsulfonylcyclopropyl)furan-2-carboxylic Acid (73 mg) as a light yellow solid. The crude was used for next step without further purification.

MS obsd. (ESI⁺) [(M+H)⁺]: 231.1.

### Intermediate Int-10

### [5-(1-methylsulfonylcyclopropyl)-1,3,4-oxadiazole-2-carbonyl]oxylithium

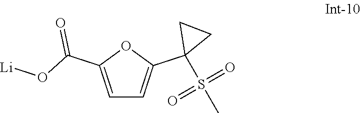

The title compound was prepared according to the following scheme:

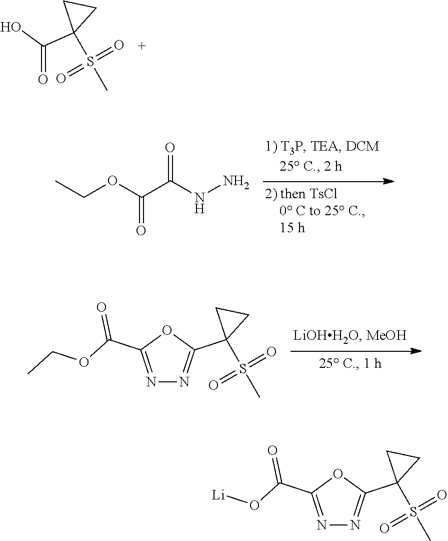

### Step 1: Preparation of ethyl 5-(1-methylsulfonylcyclopropyl)-1,3,4-oxadiazole-2-carboxylate

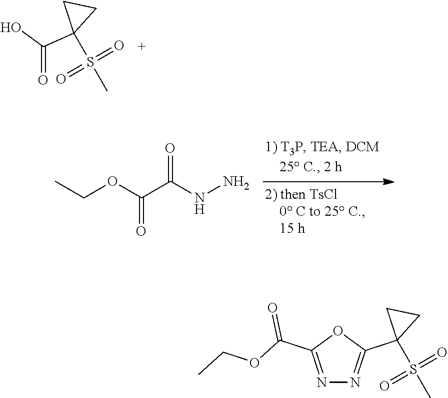

To a solution of 1-methylsulfonylcyclopropanecarboxylic acid (3.00 g, 18.3 mmol, CAS No: 1249197-58-7), ethyl 2-hydrazino-2-oxo-acetate (2.41 g, 18.3 mmol, CAS No: 35196-48-6) in DCM (100 mL) were added TEA (5.55 g, 54.8 mmol) and T₃P (11.6 g, 36.6 mmol). After stirring at room temperature for 2 h, TsCl (10.5 g, 54.8 mmol) was added at 0° C. The reaction mixture was warmed to room temperature and stirred for another 15 h. The resulting solution was concentrated in vacuum. The residue was purified by silica gel column eluted with PE/EtOAc=3/2 to afford ethyl 5-(1-methylsulfonylcyclopropyl)-1,3,4-oxadiazole-2-carboxylate (2.1 g) as yellow oil.

MS obsd. (ESI⁺) [(M+H)⁺]: 261.1. ¹H NMR (400 MHz, CDCl₃) δ ppm: 4.50 (q, J=7.2 Hz, 2H), 3.25 (s, 3H), 2.02-2.06 (m, 2H), 1.73-1.79 (m, 2H), 1.43 (t, J=7.2 Hz, 3H).

### Step 2: Preparation of [5-(1-methylsulfonylcyclopropyl)-1,3,4-oxadiazole-2-carbonyl]oxylithium (Int-10)

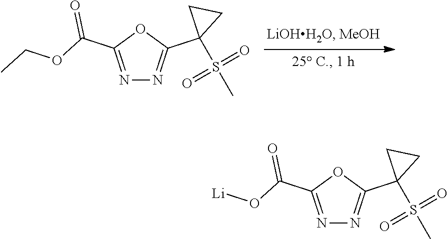

A solution of ethyl 5-(1-methylsulfonylcyclopropyl)-1,3,4-oxadiazole-2-carboxylate (350 mg, 1.34 mmol) and LiOH·H₂O (59 mg, 1.41 mmol) in methanol (10 mL) was stirred at 25° C. for 1 h. The reaction mixture was concentrated in vacuum to afford [5-(1-methylsulfonylcyclopropyl)-1,3,4-oxadiazole-2-carbonyl]oxylithium (300 mg) as a yellow solid.

The crude was used for next step without further purification.

MS obsd. (ESI⁺) [(M+H)⁺]: 233.1.

### Intermediate Int-11

### [3-(1-methylsulfonylcyclopropyl)-1,2,4-thiadiazole-5-carbonyl]oxylithium

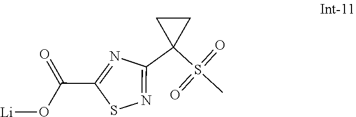

The title compound was prepared according to the following scheme:

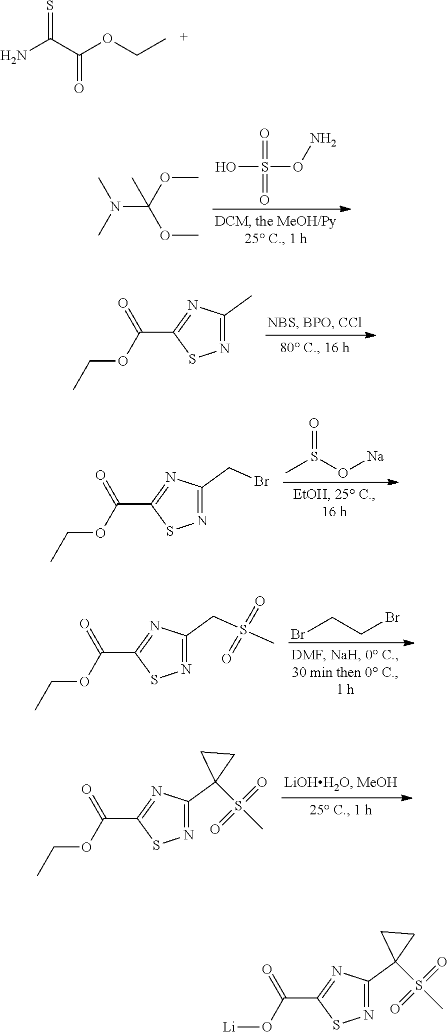

### Step 1: Preparation of ethyl 3-methyl-1,2,4-thiadiazole-5-carboxylate

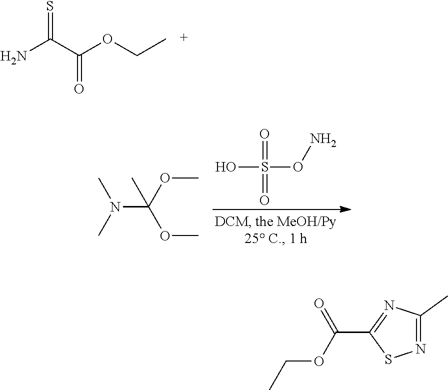

To a solution of N,N-dimethylacetamide dimethyl acetal (11.0 g, 82.6 mmol) in DCM (10 mL) was added ethyl thiooxamate (10.0 g, 75.1 mmol, CAS No: 16982-21-1). After stirred at 25° C. for 10 min, DCM was removed in vacuum. The residue was dissolved in methanol (10 mL) and then pyridine (12.2 mL, 150.2 mmol) and a solution of hydroxylamine-O-sulfonic acid (9.34 g, 82.6 mmol) in methanol (10 mL) were added at 0° C. slowly. The reaction mixture was warmed to room temperature and stirred for another 1 h. The resulting solution was concentrated in vacuum to remove methanol and the residue was dissolved in EtOAc (100 mL). The solution was washed with water (40 mL×2), brine (40 mL) and dried over Na₂SO₄. The organic layer was concentrated and the residue was purified by silica gel column eluted with PE/EtOAc=4/1 to afford ethyl 3-methyl-1,2,4-thiadiazole-5-carboxylate (8.18 g) as orange oil.

MS obsd. (ESI⁺) [(M+H)⁺]: 173.1.

### Step 2: Preparation of ethyl 3-(bromomethyl)-1,2,4-thiadiazole-5-carboxylate

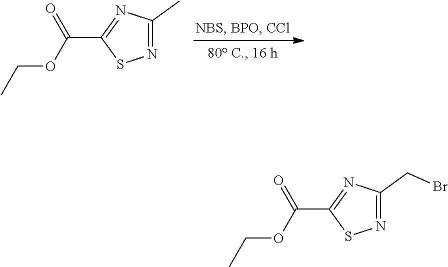

To a solution of ethyl 3-methyl-1,2,4-thiadiazole-5-carboxylate (10.0 g, 58.1 mmol) in CCl₄ (82 mL) were added NBS (12.0 g, 69.7 mmol) and BPO (21.1 g, 87.1 mmol) at 25° C. The reaction mixture was stirred at 80° C. for 16 h. The resulting solution was concentrated to remove CCl₄. The residue was re-dissolved in EtOAc (100 mL) and washed with water (40 mL×2) and brine (40 mL). The organic layer was dried over Na₂SO₄ and concentrated in vacuum. The residue was purified by silica gel column eluted with PE/EtOAc=4/1 to afford ethyl 3-(bromomethyl)-1,2,4-thiadiazole-5-carboxylate (7.26 g) as orange oil.

MS obsd. (ESI⁺) [(M+H)⁺]: 250.9.

### Step 3: Preparation of ethyl 3-(methylsulfonylmethyl)-1,2,4-thiadiazole-5-carboxylate

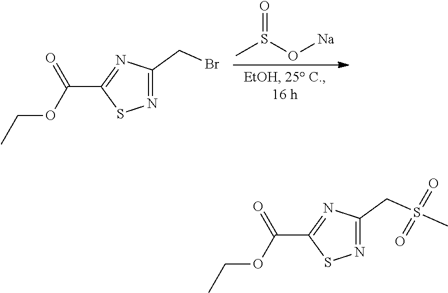

To a solution of ethyl 3-(bromomethyl)-1,2,4-thiadiazole-5-carboxylate (7.69 g, 30.6 mmol) in ethanol (120 mL) was added sodium methanesulfinate (4.69 g, 45.9 mmol) at 25° C. The reaction mixture was stirred at the same temperature for 16 h. The resulting solution was concentrated in vacuum. The residue was dissolved in EtOAc (100 mL), washed with water (40 ml×2) and brine (40 mL). The organic layer was dried over Na₂SO₄ and concentrated in vacuum. The residue was purified by silica gel column eluted with PE/EtOAc=2/3 to afford ethyl 3-(methylsulfonylmethyl)-1,2,4-thiadiazole-5-carboxylate (6.12 g) as a white solid.

MS obsd. (ESI⁺) [(M+H)⁺]: 251.1.

### Step 4: Preparation of ethyl 3-(1-methylsulfonylcyclopropyl)-1,2,4-thiadiazole-5-carboxylate

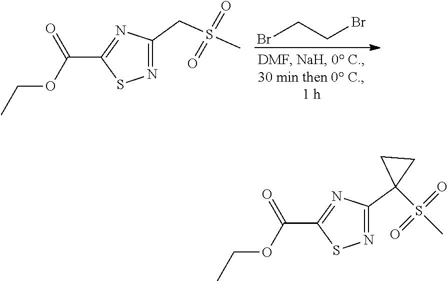

To a solution of ethyl 3-(methylsulfonylmethyl)-1,2,4-thiadiazole-5-carboxylate (500 mg, 2.00 mmol) in DMF (10 mL) was added sodium hydride in several portions (120 mg, 4.99 mmol, 60% in oil) under nitrogen atmosphere at 0° C. After stirred at 0° C. for 30 min, 1,2-dibromoethane (563 mg, 3.00 mmol) was added dropwise. The reaction mixture was stirred at 0° C. for 1 h. The resulted solution was diluted with EtOAc (100 mL) at 0° C. and then ice-water (50 mL). The organic layer was washed with brine (50 mL), dried over Na₂SO₄ and concentrated in vacuum. The residue was purified by silica gel column eluted with PE/EtOAc=7/3 to afford ethyl 3-(1-methylsulfonylcyclopropyl)-1,2,4-thiadiazole-5-carboxylate (298 mg) as a white solid.

MS obsd. (ESI⁺) [(M+H)⁺]: 277.0. ¹H NMR (400 MHz, CDCl₃) δ ppm: 4.52 (q, J=7.2 Hz, 2H), 3.37 (s, 3H), 1.98-2.04 (m, 2H), 1.81-1.87 (m, 2H), 1.45 (t, J=7.2 Hz, 3H).

### Step 5: Preparation of [3-(1-methylsulfonylcyclopropyl)-1,2,4-thiadiazole-5-carbonyl]oxylithium (Int-11)

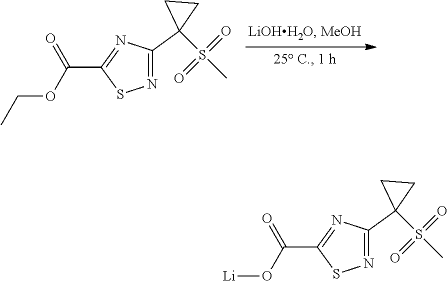

To a solution of ethyl 3-(1-methylsulfonylcyclopropyl)-1,2,4-thiadiazole-5-carboxylate (298 mg, 1.1 mmol) in MeOH (10 mL) was added LiOH·H₂O (88 mg, 1.1 mmol) and 1 drop of water. The reaction was stirred at 25° C. for 2 h. The resulting solution was concentrated in vacuum to give [3-(1-methylsulfonylcyclopropyl)-1,2,4-thiadiazole-5-carbonyl]oxylithium (279 mg) as a white solid. The crude was used for next step without further purification.

MS obsd. (ESI⁺) [(M+H)⁺]: 255.0.

### Intermediate Int-12

### 5-cyclopropylsulfonylfuran-2-carboxylic Acid

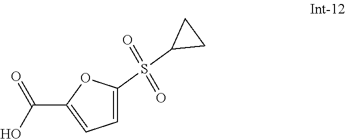 [SMILES: O=C(O)c1ccc(S(=O)(=O)C2CC2)o1]

The title compound was prepared according to the following scheme:

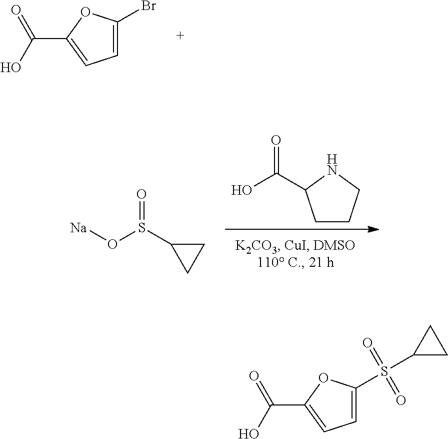

A mixture of 5-bromo-2-furoic acid (7.45 g, 39.0 mmol), cyclopropylsulfinyloxysodium (5.0 g, 39.0 mmol, CAS No: 910209-21-1), 2-pyrrolidinecarboxylic acid (1.8 g, 15.6 mmol), K₂CO₃ (2.16 g, 15.6 mmol) and CuI (2.97 g, 15.6 mmol) in DMSO (60 mL) was stirred at 110° C. for 21 h. The reaction mixture was acidified by HCl (5M) to adjust pH=3-4 and then extracted with EtOAc (50 mL×6). The combined organic layers were washed with brine (50 mL×3), dried over Na₂SO₄ and concentrated in vacuum. The residue was purified by silica gel column eluted with PE/EtOAc=3/2 to afford 5-cyclopropylsulfonylfuran-2-carboxylic acid (4.92 g) as a yellow solid.

MS obsd. (ESI⁺) [(M+H)⁺]: 217.0.

### Intermediate Int-13

### 5-(1-methylsulfonylpropyl)furan-2-carboxylic Acid

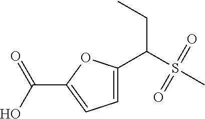

The title compound was prepared in analogy to the procedure described for the preparation of 5-(1-methylsulfonylcyclopropyl)furan-2-carboxylic acid (Int-9), by using iodoethane instead of 1,2-dibromoethane.

### Intermediates Int-14 & Int-15

### 5-(1-methyl-1-methylsulfonyl-ethyl)furan-2-carboxylic acid and 5-(1-methylsulfonylethyl)furan-2-carboxylic Acid

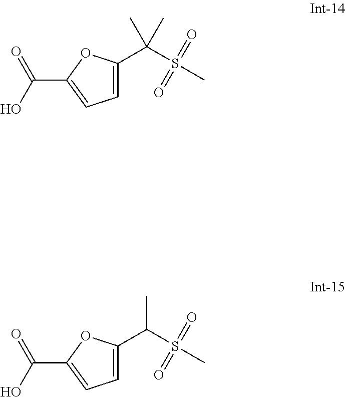

The title compounds Int-14 and Int-15 were prepared in analogy to the procedure described for the preparation of 5-(1-methylsulfonylcyclopropyl)furan-2-carboxylic acid (Int-9), by using iodomethane instead of 1,2-dibromoethane.

### Intermediate Int-16

### 5-[bromo(methylsulfonyl)methyl]furan-2-carboxylic Acid

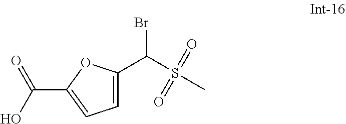

The title compound was prepared according to the following scheme:

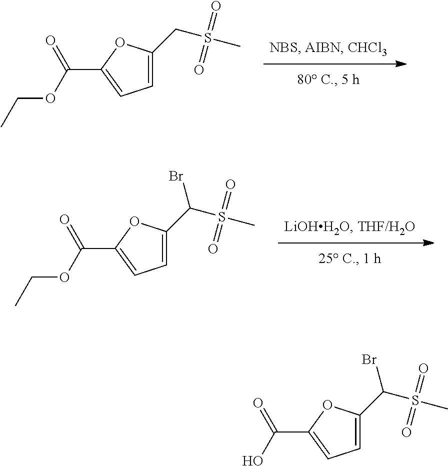 [SMILES: CCOC(=O)c1ccc(CS(C)(=O)=O)o1 ; CCOC(=O)c1ccc(C(Br)S(C)(=O)=O)o1 ; CS(=O)(=O)C(Br)c1ccc(C(=O)O)o1]

### Step 1: Preparation of ethyl 5-[bromo(methylsulfonyl)methyl]furan-2-carboxylate

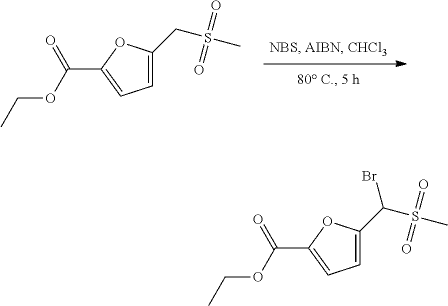 [SMILES: CCOC(=O)c1ccc(CS(C)(=O)=O)o1 ; CCOC(=O)c1ccc(C(Br)S(C)(=O)=O)o1]

To a solution of ethyl 5-(methylsulfonylmethyl)furan-2-carboxylate (20.0 g, 86.1 mmol) and AIBN (1.41 g, 8.61 mmol) in CHCl₃ (500 mL) was added NBS (18.4 g, 103.3 mmol). The reaction mixture was stirred at 80° C. for 5 h. After cooling to room temperature, the resulting solution was concentrated in vacuum. The residue was purified by silica gel column eluted with PE/EtOAc=4/1 to afford ethyl 5-[bromo(methylsulfonyl)methyl]furan-2-carboxylate (8.5 g) as a light yellow solid.

MS obsd. (ESI⁺) [(M+Na)+]: 333.0.

### Step 2: Preparation of ethyl 5-[bromo(methylsulfonyl)methyl]furan-2-carboxylic Acid (Int-16)

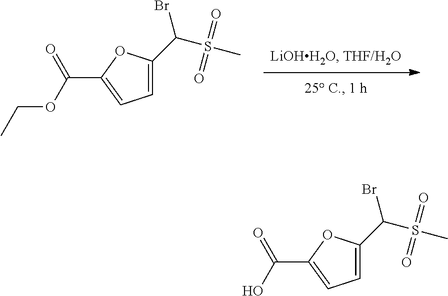 [SMILES: CCOC(=O)c1ccc(C(Br)S(C)(=O)=O)o1 ; CS(=O)(=O)C(Br)c1ccc(C(=O)O)o1]

To a solution of ethyl 5-[bromo(methylsulfonyl)methyl]furan-2-carboxylate (3000 mg, 9.64 mmol) in THF (30 mL) and water (10 mL) was added LiOH·H₂O (462 mg, 19.3 mmol). The reaction mixture was stirred at 25° C. for 1 h. The reaction mixture was concentrated in vacuum to remove the THF. The resulting solution was adjusted to pH=2-4 with aqueous HCl solution (2 M) and extracted with DCM (50 mL×3). The combined organic layers were washed with brine (50 mL), dried over Na₂SO₄ and concentrated to afford 5-[bromo(methylsulfonyl)methyl]furan-2-carboxylic acid (1000 mg) as a yellow solid.

MS obsd. (ESI⁺) [(M+H)⁺]: 305.0.

### Intermediate Int-17

### 5-(2-amino-1-methylsulfonyl-2-oxo-ethyl)furan-2-carboxylic Acid

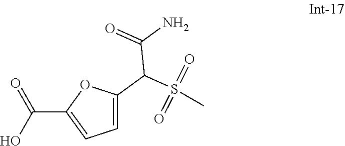 [SMILES: CS(=O)(=O)C(C(N)=O)c1ccc(C(=O)O)o1]

The title compound was prepared according to the following scheme:

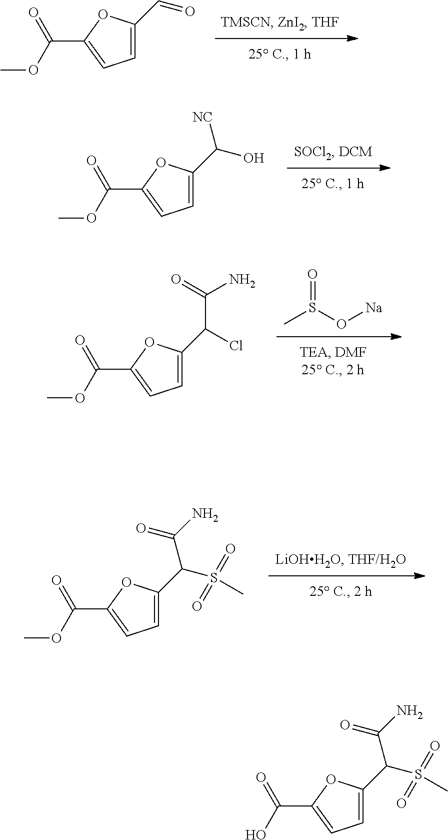

### Step 1: Preparation of methyl 5-[cyano(hydroxy)methyl]furan-2-carboxylate

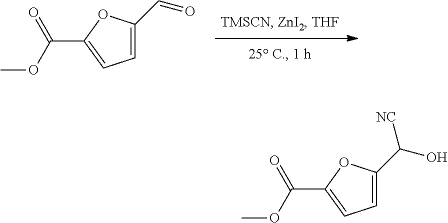

To a solution of methyl 5-formylfuran-2-carboxylate (42 mg, 0.270 mmol) in THF (5 mL) was added ZnI₂ (4.4 mg, 0.010 mmol) and then TMSCN (0.04 mL, 0.330 mmol) dropwise at 0° C. After warming to 25° C., the mixture was stirred for 1 h. The resulting solution was diluted with water (20 mL) and acidified with HCl (3 M) to pH=3. The mixture was extracted with diisopropylether (10 mL×3). The organic phase was dried over Na₂SO₄ and concentrated in vacuum to afford methyl 5-[cyano(hydroxy)methyl]furan-2-carboxylate (45 mg) as light yellow oil.

MS obsd. (ESI⁺) [(M+H)⁺]: 182.1.

### Step 2: Preparation of methyl 5-(2-amino-1-chloro-2-oxo-ethyl)furan-2-carboxylate

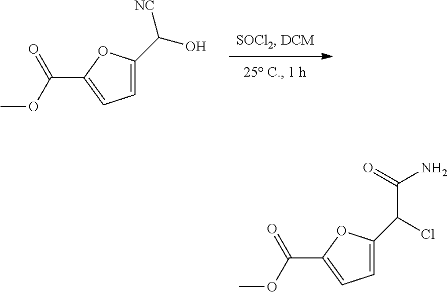

To a solution of methyl 5-[cyano(hydroxy)methyl]furan-2-carboxylate (182 mg, 1.0 mmol) in DCM (8 mL) was added SOCl₂ (0.08 mL, 1.1 mmol) at 0° C. The mixture was stirred at 25° C. for 1 h. The resulting solution was concentrated in vacuum. The residue was purified by prep-TLC (PE/EtOAc=4/1) to give methyl 5-(2-amino-1-chloro-2-oxo-ethyl)furan-2-carboxylate (80 mg) as light yellow oil.

MS obsd. (ESI⁺) [(M+H)⁺]: 218.1.

### Step 3: Preparation of methyl 5-(2-amino-1-methylsulfonyl-2-oxo-ethyl)furan-2-carboxylate

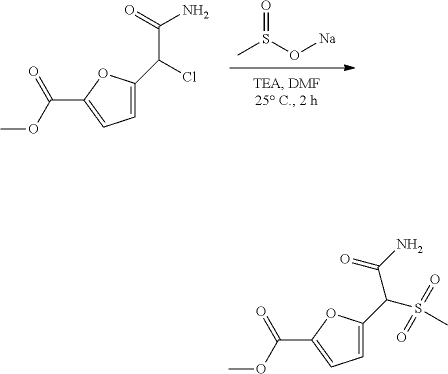

To a solution of methyl 5-(2-amino-1-chloro-2-oxo-ethyl)furan-2-carboxylate (50 mg, 0.23 mmol) in DMF (2 mL) were added sodium methanesulphinate (28.15 mg, 0.28 mmol) and TEA (0.04 mL, 0.28 mmol). The reaction was stirred at 25° C. for 2 h. The resulted solution was diluted with EtOAc (30 mL) and washed with water (20 mL×2). The organic layer was dried over Na₂SO₄ and concentrated in vacuum. The residue was purified by silica gel column eluted with PE/EtOAc=1/1 to afford methyl 5-(2-amino-1-methylsulfonyl-2-oxo-ethyl)furan-2-carboxylate (60 mg) as a brown solid.

MS obsd. (ESI⁺) [(M+H)⁺]: 262.0.

### Step 4: Preparation of 5-(2-amino-1-methylsulfonyl-2-oxo-ethyl)furan-2-carboxylic acid (Int-17)

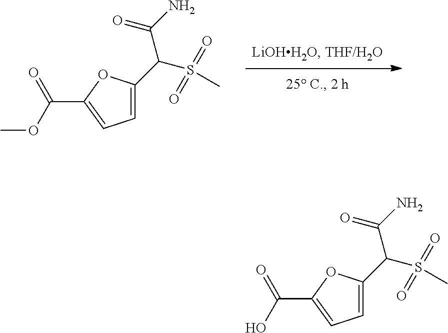

To a solution of methyl 5-(2-amino-1-methylsulfonyl-2-oxo-ethyl)furan-2-carboxylate (80 mg, 0.31 mmol) in THF (3 mL) and water (0.5 mL) was added LiOH·H₂O (9 mg, 0.37 mmol). The reaction was stirred at 25° C. for 2 h. The resulted solution was diluted with water (20 mL), adjusted to pH=5 with HCl (2 M) and extracted with EtOAc (20 mL×2). The combined organic layers were dried over Na₂SO₄ and concentrated to afford the crude product 5-(2-amino-1-methylsulfonyl-2-oxo-ethyl)furan-2-carboxylic acid (50 mg) as colorless oil.

MS obsd. (ESI⁺) [(M+H)⁺]:248.0.

### Intermediate Int-18

### [2-(methylsulfonylmethyl)oxazole-5-carbonyl]oxylithium

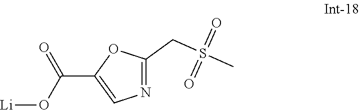

The title compound was prepared according to the following scheme:

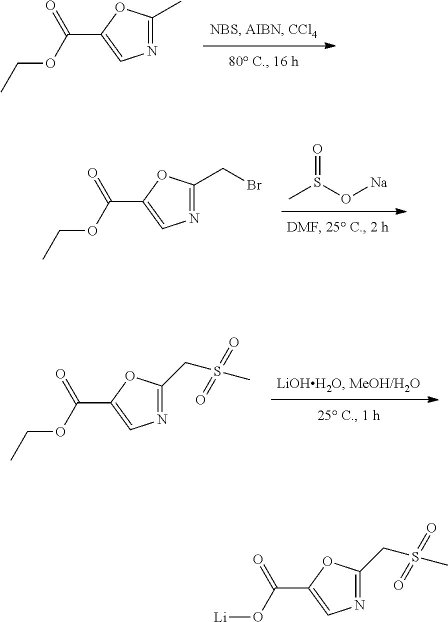

### Step 1: Preparation of ethyl 2-(bromomethyl)oxazole-5-carboxylate

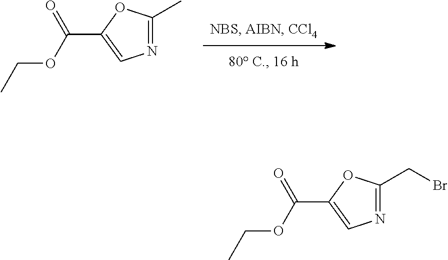

A mixture of ethyl 2-methyloxazole-5-carboxylate (2.5 g, 16.1 mmol), NBS (4.3 g, 24.2 mmol) and AIBN (1.06 g, 6.45 mmol) in CCl₄ (50 mL) was stirred at 80° C. for 16 h. The reaction mixture was filtered and the filtrate was concentrated in vacuum. The residue was purified by silica gel column eluted with PE/EtOAc=9/1 to afford ethyl 2-(bromomethyl)oxazole-5-carboxylate (3.77 g) as a light yellow solid.

MS obsd. (ESI⁺) [(M+H)⁺]: 234.0.

### Step 2: Preparation of ethyl 2-(methylsulfonylmethyl)oxazole-8_carboxylate

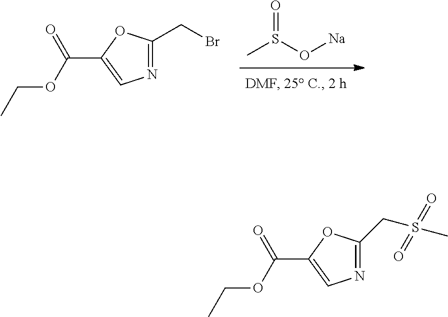

A mixture of ethyl 2-(bromomethyl)oxazole-5-carboxylate (3.77 g, 16.1 mmol) and sodium methanesulfinate (2.47 g, 24.2 mmol) in DMF (15 mL) was stirred at 25° C. for 2 h. The mixture was concentrated in vacuum and the residue was purified by silica gel column eluted with PE/EtOAc=7/3 to afford ethyl 2-(methylsulfonylmethyl)oxazole-5-carboxylate (600 mg) as a white solid.

MS obsd. (ESI⁺) [(M+H)⁺]: 234.1.

### Step 3: Preparation of [2-(methylsulfonylmethyl)oxazole-5-carbonyl]oxylithium (Int-18)

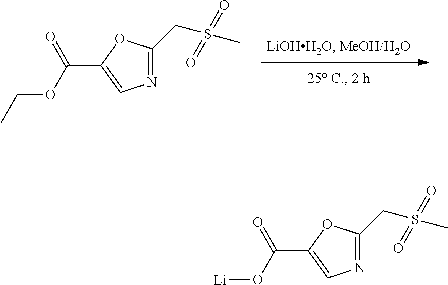

A mixture of ethyl 2-(methylsulfonylmethyl)oxazole-5-carboxylate (50 mg, 0.21 mmol) and LiOH H₂O (18 mg, 0.43 mmol) in methanol (5 mL) and water (0.1 mL) was stirred at 25° C. for 2 h. After that, the mixture was concentrated in vacuum to afford [2-(methylsulfonylmethyl)oxazole-5-carbonyl]oxylithium (44 mg) as a light red solid. The crude was used for in next step without further purification.

MS obsd. (ESI⁺) [(M+H)⁺]: 206.0.

### Intermediates Int-19 & Int-20

### 2-(1-methylsulfonylethyl)oxazole-5-carboxylic acid and 2-(1-methyl-1-methylsulfonyl-ethyl)oxazole-5-carboxylic Acid

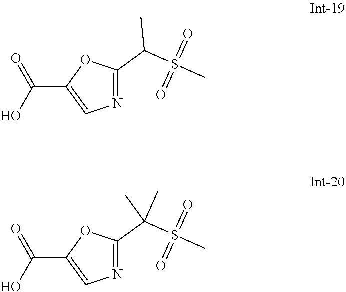

The title compounds Int-19 and Int-20 were prepared in analogy to the procedure described for the preparation of 5-(1-methylsulfonylcyclopropyl)furan-2-carboxylic acid (Int-9), by using iodomethane instead of 1,2-dibromoethane and ethyl 2-(methylsulfonylmethyl)oxazole-5-carboxylate instead of ethyl 5-(methylsulfonylmethyl)furan-2-carboxylate.

### Intermediate Int-21

### 2-(1-methylsulfonylethyl)-4,5-dihydrothiazole-4-carboxylic Acid

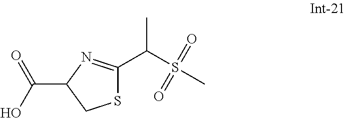

The title compound was prepared according to the following s e:

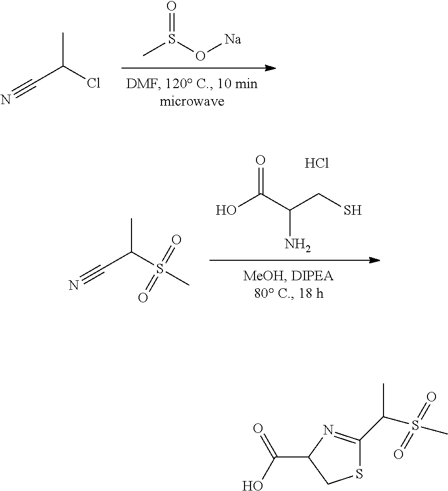

### Step 1: Preparation of 2-methylsulfonylpropanenitrile

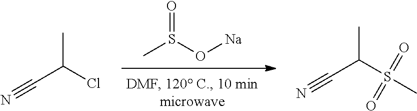

To a solution of 2-chloropropanenitrile (1.0 g, 11.2 mmol, CAS No: 1617-17-0) in DMF (5 ml) was added sodium methanesulfinate (1.3 g, 12.3 mmol) was added. Then the reaction was stirred at 120° C. for 10 min under microwave. The reaction mixture was concentrated in vacuum to afford the crude product 2-methylsulfonylpropanenitrile (0.36 g) as brown oil.

### Step 2: Preparation of 2-(1-methylsulfonylethyl)-4,5-dihydrothiazole-4-carboxylic Acid (Int-21)

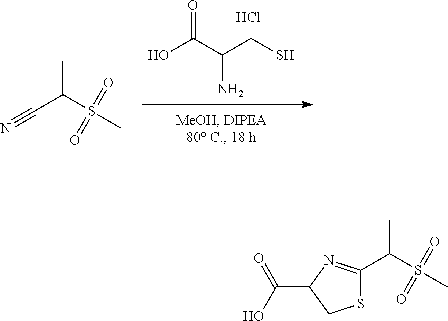

To a mixture of cysteine hydrochloride (592 mg, 3.75 mmol) and 2-methylsulfonylpropanenitrile (500 mg, 3.75 mmol) in MeOH (5 ml) was added DIPEA (1.97 mg, 11.3 mmol). Then the mixture was stirred at 80° C. for 18 h. The solvent was removed and the residue was purified by prep-HPLC to afford 2-(1-methylsulfonylethyl)-4,5-dihydrothiazole-4-carboxylic acid (463 mg) as a white solid.

### Intermediate Int-22

### 2-(1-methylsulfonylcyclopropyl)thiazole-5-carboxylic Acid

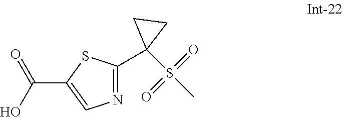

The title compound was prepared according to the following scheme:

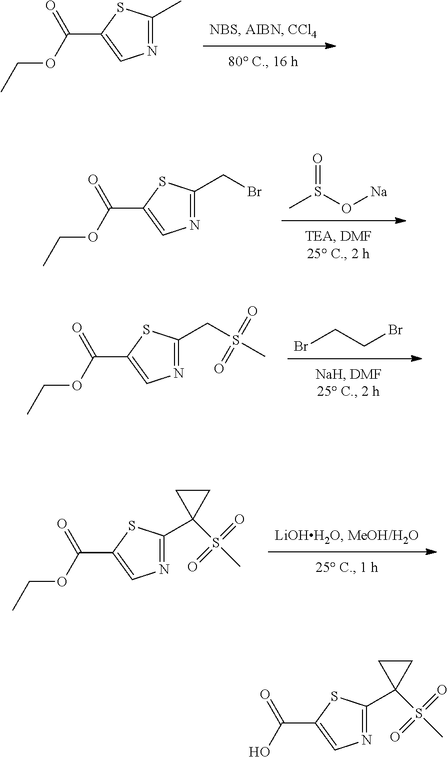

### Step 1: Preparation of ethyl 2-(bromomethyl)thiazole-5-carboxylate

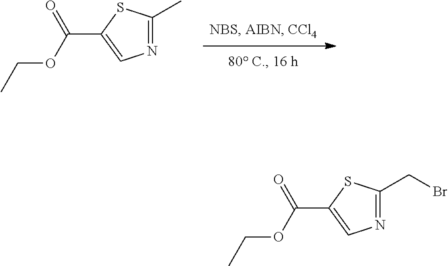

To a solution of ethyl 2-methylthiazole-5-carboxylate (1.0 g, 5.84 mmol) in CCl₄ (20 ML) were added NBS (1.04 g, 5.84 mmol) and AIBN (1.41 g, 5.84 mmol). The reaction was stirred at 80° C. for 3 h. The resulting solution was concentrated in vacuum. The residue was purified by silica gel column eluted with PE/EtOAc=20/1 to afford ethyl 2-(bromomethyl)thiazole-5-carboxylate (960 mg) as orange oil.

MS obsd. (ESI⁺) [(M+H)⁺]:250.0.

### Step 2: Preparation of ethyl 2-(methylsulfonylmethyl)thiazole-5-carboxylate

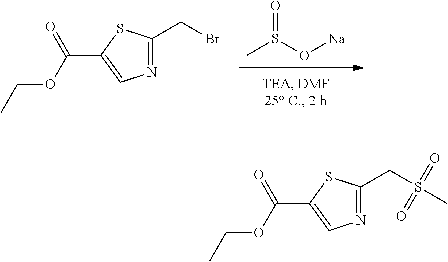

To a solution of ethyl 2-(bromomethyl)thiazole-5-carboxylate (2.1 g, 8.4 mmol) in DMF (60 mL) were added TEA (1.4 mL, 10.1 mmol) and sodium methanesulfinate (1.03 g, 10.1 mmol). The reaction was stirred at 25° C. for 2 h. The resulting mixture was diluted with EtOAc (60 mL) and washed with water (20 mL×2) and brine (20 mL×2). The organic layer was dried over Na₂SO₄ and concentrated in vacuum. The residue was purified by silica gel column eluted with PE/EtOAc=1/1 to afford ethyl 2-(methylsulfonylmethyl)thiazole-5-carboxylate (1.3 g) as yellow oil.

MS obsd. (ESI⁺) [(M+H)⁺]: 250.0

### Step 3: Preparation of ethyl 2-(1-methylsulfonylcyclopropyl)thiazole-5-carboxylate

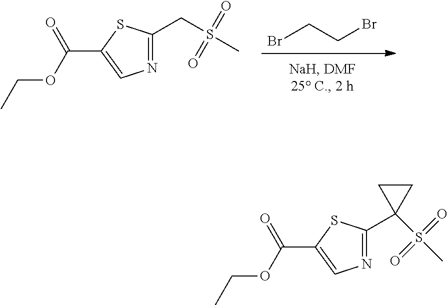

To a solution of ethyl 2-(methylsulfonylmethyl)thiazole-5-carboxylate (200 mg, 0.80 mmol) in DMF (5 mL) were added sodium hydride (85 mg, 2.13 mmol, 60% in oil) and 1,2-dibromoethane (0.09 mL, 1.02 mmol). The reaction was stirred at 25° C. for 2 h. The resulted mixture was diluted with EtOAc (60 mL) and washed with water (20 mL×2) and brine (20 mL×2). The organic layer was dried over Na₂SO₄ and concentrated in vacuum. The residue was purified by reversed flash column (eluting with H₂O/ACN=95/5) to afford ethyl 2-(1-methylsulfonylcyclopropyl)thiazole-5-carboxylate (96 mg) as a white solid.

MS obsd. (ESI⁺) [(M+H)⁺]: 276.0

### Step 4: Preparation of 2-(1-methylsulfonylcyclopropyl)thiazole-5-carboxylic Acid (Int-22)

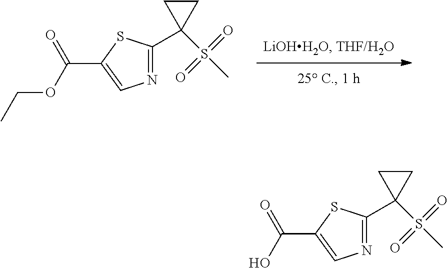 [SMILES: CCOC(=O)c1cnc(C2(S(C)(=O)=O)CC2)s1 ; CS(=O)(=O)C1(c2ncc(C(=O)O)s2)CC1]

To a solution of ethyl ethyl 2-(1-methylsulfonylcyclopropyl)thiazole-5-carboxylate (96 mg, 0.349 mmol) in THF (5 mL) and water (1 mL) was added LiOH·H₂O (20 mg, 0.480 mmol). The reaction was stirred at 25° C. for 2 h. The resulting solution was diluted with water (20 mL), adjusted to pH=6 with HCl (2 M) and extracted with EtOAc (20 mL×2). The combined organic layers were dried over Na₂SO₄ and concentrated in vacuum to afford 2-(1-methylsulfonylcyclopropyl)thiazole-5-carboxylic acid (80 mg) as a brown solid.

MS obsd. (ESI⁺) [(M+H)⁺]: 248.0.

### Intermediate Int-23

### 2-(1-methyl-1-methylsulfonyl-ethyl)thiazole-5-carboxylic Acid

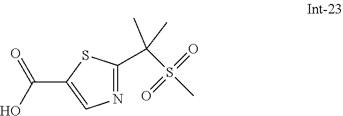

The title compound was prepared in analogy to the procedure described for the preparation of 2-(1-methylsulfonylcyclopropyl)thiazole-5-carboxylic acid (Int-22), by using iodomethane instead of 1,2-dibromoethane.

### Intermediate Int-24

### 3-(methylsulfonylmethyl)isothiazole-5-carboxylic Acid

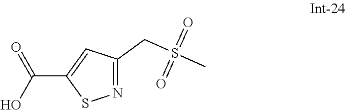

The title compound was prepared in analogy to the procedure described for the preparation of [2-(methylsulfonylmethyl)oxazole-5-carbonyl]oxylithium (Int-18), by using methyl 3-methylisothiazole-5-carboxylate instead of ethyl 2-methyloxazole-5-carboxylate.

### Intermediate Int-25

### 3-(1-methyl-1-methylsulfonyl-ethyl)isothiazole-5-carboxylic Acid

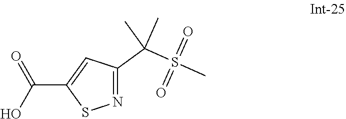

The title compound was prepared in analogy to the procedure described for the preparation of 2-(1-methylsulfonylcyclopropyl)thiazole-5-carboxylic acid (Int-22), by using methyl 3-methylisothiazole-5-carboxylate instead of ethyl 2-methylthiazole-5-carboxylate and iodomethane instead of ethylene dibromide.

### Intermediate Int-26

### 5-(1-methyl-1-methylsulfonyl-ethyl)isothiazole-3-carboxylic Acid

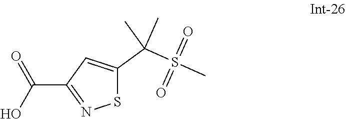

The title compound was prepared in analogy to the procedure described for the preparation of 2-(1-methylsulfonylcyclopropyl)thiazole-5-carboxylic acid (Int-22), by using methyl 5-methylisothiazole-3-carboxylate instead of ethyl 2-methylthiazole-5-carboxylate and iodomethane instead of 1,2-dibromoethane.

### Intermediate Int-27

### 5-(1-methylsulfonylcyclopropyl)isothiazole-3-carboxylic Acid

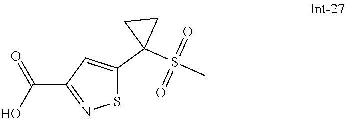

The title compound was prepared in analogy to the procedure described for the preparation of 2-(1-methylsulfonylcyclopropyl)thiazole-5-carboxylic acid (Int-22), by using methyl 5-methylisothiazole-3-carboxylate instead of ethyl 2-methylthiazole-5-carboxylate.

### Intermediate Int-28

### 3-(methylsulfonylmethyl)-1,2,4-thiadiazole-5-carboxylic Acid

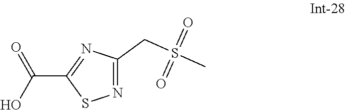

The title compound was prepared according to the following scheme:

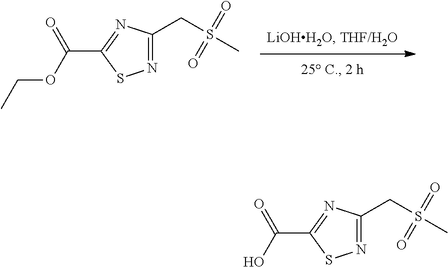

To a solution of ethyl 3-(methylsulfonylmethyl)-1,2,4-thiadiazole-5-carboxylate (62 mg, 0.25 mmol, CAS No: 2283137-86-8) in THF (2 mL) and Water (1 mL) was added LiOH·H₂O (16 mg, 0.37 mmol). The reaction mixture was stirred at 25° C. for 2 h. The resulting solution was diluted with water (30 mL), adjusted to pH=5 with hydrochloric acid (2 M) and extracted with EtOAc (20 mL×3). The combined organic layers were dried over Na₂SO₄ and concentrated in vacuum. The residue was purified by silica gel column eluted with DCM/MeOH=20/1 to afford 3-(methylsulfonylmethyl)-1,2,4-thiadiazole-5-carboxylic acid (51 mg) as a white solid.

MS obsd. (ESI⁺) [(M+H)⁺]: 223.1.

### Intermediates Int-29 & Int-30

### 3-(1-methylsulfonylethyl)-1,2,4-thiadiazole-5-carboxylic Acid and 3-(1-methyl-1-methylsulfonyl-ethyl)-1,2,4-thiadiazole-5-carboxylic Acid

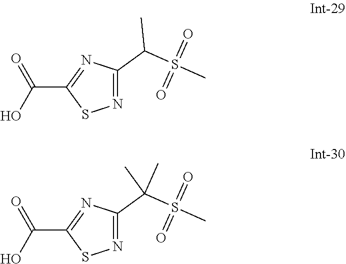

The title compounds were prepared in analogy to the procedure described for the preparation of [3-(1-methylsulfonylcyclopropyl)-1,2,4-thiadiazole-5-carbonyl]oxylithium (Int-11), by using iodomethane instead of 1,2-dibromoethane.

### Intermediate Int-31

### [1-(methylsulfonylmethyl)pyrazole-3-carbonyl]oxylithium

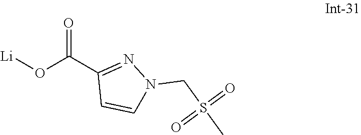

The title compound was prepared according to the following scheme:

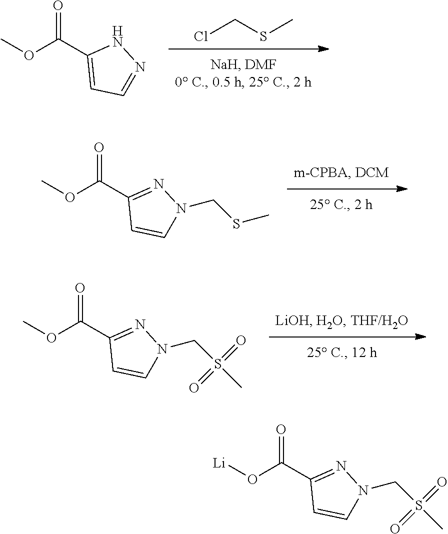

### Step 1: Preparation of methyl 1-(methylsulfanylmethyl)pyrazole-3-carboxylate

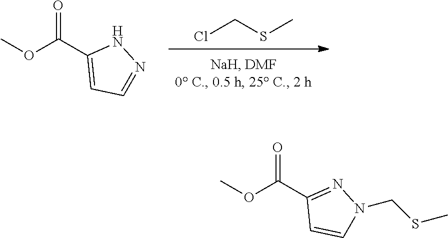

To a solution of methyl 1H-pyrazole-5-carboxylate (1100 mg, 8.72 mmol, CAS No: 15366-34-4) in DMF (15 mL) was added sodium hydride (251 mg, 10.5 mmol, 60% in oil) at 0° C. After stirred at 0° C. for 30 min, chloromethyl methyl sulfide (1011 mg, 10.5 mmol, CAS No: 2373-51-5) was added. The reaction mixture was warmed to room temperature and stirred for another 2 h. The reaction was quenched with saturated aqueous NH₄Cl solution. The resulted solution was diluted with EtOAc (100 mL) and washed with brine (50 mL×3). The organic layer was dried over anhydrous Na₂SO₄ and concentrated in vacuum. The residue was purified by silica gel column eluted with PE/EtOAc=3/2 to afford methyl 1-(methylsulfanylmethyl)pyrazole-3-carboxylate (417 mg) as light yellow oil.

MS obsd. (ESI⁺) [(M+H)⁺]: 187.1. ¹H NMR (400 MHz, DMSO-d₆) δ ppm: 7.98 (d, J=2.4 Hz, 1H), 6.81 (d, J=2.4 Hz, 1H), 5.36 (s, 2H), 3.81 (s, 3H), 2.13 (s, 3H).

### Step 2: Preparation of methyl 1-(methylsulfonylmethyl)pyrazole-3-carboxylate

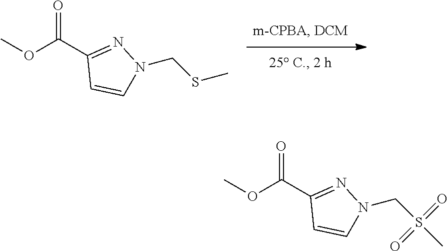

A mixture of methyl 1-(methylsulfanylmethyl)pyrazole-3-carboxylate (417 mg, 2.24 mmol) and m-CPBA (1159 mg, 6.72 mmol) in DCM (10 mL) was stirred at 25° C. for 2 h. The reaction was diluted with DCM (50 mL) and washed with brine (50 mL×3). The organic layer was dried over Na₂SO₄ and concentrated in vacuum. The residue was purified by silica gel column eluted with PE/EtOAc=1/1 to afford methyl 1-(methylsulfonylmethyl)pyrazole-3-carboxylate (347 mg) as a white solid.

MS obsd. (ESI+) [(M+H)+]: 219.1.

### Step 3: Preparation of [1-(methylsulfonylmethyl)pyrazole-3-carbonyl]oxylithium (Int-31)

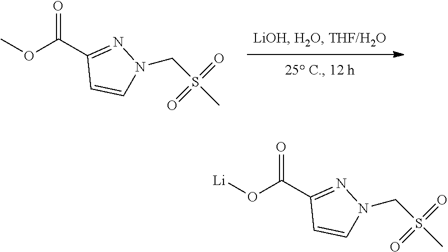

To a solution of methyl 1-(methylsulfonylmethyl)pyrazole-3-carboxylate (30 mg, 0.140 mmol) in mixed solution of THF (2.5 mL) and water (0.5 mL) was added LiOH·H₂O (7 mg, 0.160 mmol). The reaction was stirred at 25° C. for 12 h and then concentrated in vacuum to afford [1-(methylsulfonylmethyl)pyrazole-3-carbonyl]oxylithium (20 mg) as a light yellow solid. The crude was used for next step without further purification.

MS obsd. (ESI⁺) [(M+H)⁺]: 205.1

### Intermediate Int-32

### [1-(1-methylsulfonylethyl)pyrazole-3-carbonyl]oxylithium

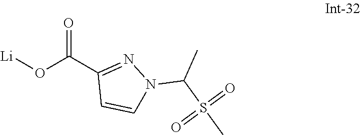

The title compound was prepared according to the following scheme:

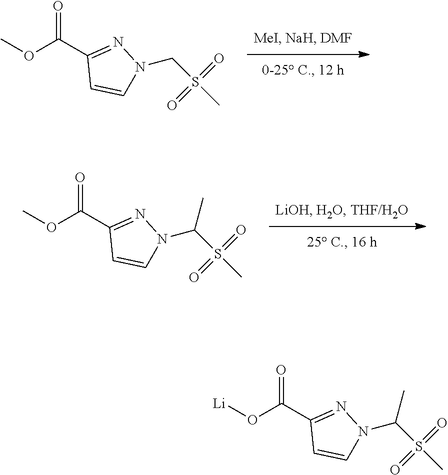

### Step 1: Preparation of methyl 1-(1-methylsulfonylethyl)pyrazole-3-carboxylate

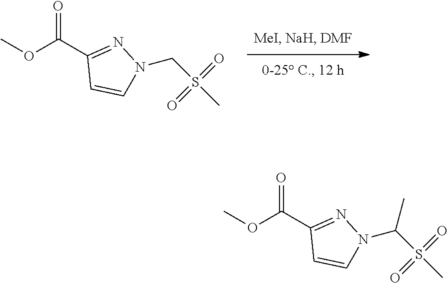

To a solution of methyl 1-(methylsulfonylmethyl)pyrazole-3-carboxylate (100 mg, 0.460 mmol) in DMF (3 mL) were added sodium hydride (33 mg, 1.38 mmol, 60% in oil) and then iodomethane (0.05 mL, 0.850 mmol) at 0° C. The reaction was stirred at 25° C. for 12 h under N₂ atmosphere and then quenched with saturated aqueous NH₄Cl (50 mL) solution. The resulted solution was extracted with EtOAc (15 mL×3). The combined organic layers were washed with brine (20 mL), dried over anhydrous Na₂SO₄ and concentrated in vacuum to afford methyl 1-(1-methylsulfonylethyl)pyrazole-3-carboxylate (100 mg) as a white solid.

MS obsd. (ESI⁺) [(M+H)⁺]: 233.1.

### Step 2: Preparation of [1-(1-methylsulfonylethyl)pyrazole-3-carbonyl]oxylithium (Int-32)

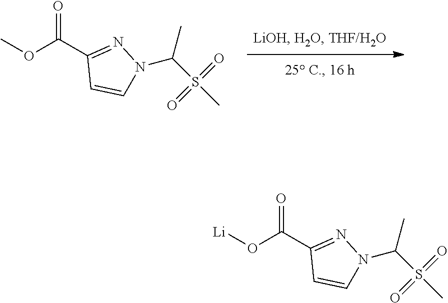

To a solution of methyl 1-(1-(methylsulfonyl)ethyl)-1H-pyrazole-3-carboxylate (100 mg, 0.5 mmol) in THF (3 mL) and water (1 mL) was added LiOH·H₂O (23 mg). The reaction was stirred at 25° C. for 16 h and then concentrated in vacuum to give [1-(1-methylsulfonylethyl)pyrazole-3-carbonyl]oxylithium (93.5 mg) as a light yellow solid. The crude was used for next step without further purification.

MS obsd. (ESI⁺) [(M+H)⁺]: 219.1.

### Intermediate Int-33

### 3-(methylsulfonylmethyl)-1,2,4-oxadiazole-5-carboxylic Acid

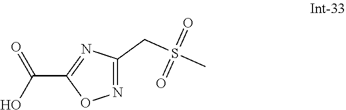

The title compound was prepared in analogy to the procedure described for the preparation of [2-(methylsulfonylmethyl)oxazole-5-carbonyl]oxylithium (Int-18), by using ethyl 3-(chloromethyl)-1,2,4-oxadiazole-5-carboxylate instead of ethyl 2-(bromomethyl)oxazole-5-carboxylate.

### Intermediate Int-34

### [2-(methylsulfonylmethyl)triazole-4-carbonyl]oxylithium

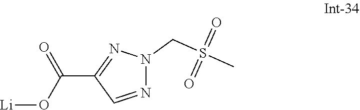

The title compound was prepared in analogy to the procedure described for the preparation of [1-(methylsulfonylmethyl)pyrazole-3-carbonyl]oxylithium (Int-31), by using methyl 1H-triazole-4-carboxylate instead of methyl 1H-pyrazole-5-carboxylate.

### Intermediate Int-35

### 5-methylsulfanylfuran-2-carboxylic Acid

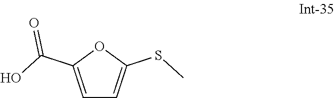

The title compound was prepared according to the following scheme:

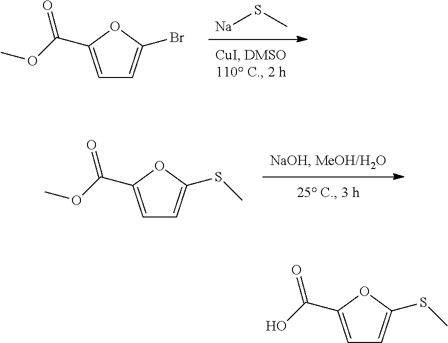

### Step 1: Preparation of methyl 5-methylsulfanylfuran-2-carboxylate

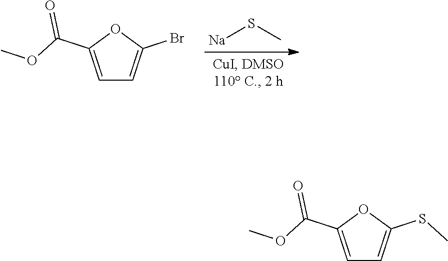

To a solution of methyl 5-bromofuran-2-carboxylate (10 g, 48.8 mmol, Eq: 1) in DMSO (20 ml) was added the sodium thiomethoxide (6.84 g, 97.6 mmol) followed by CuI (10.2 g, 53.7 mmol). The mixture was then heated to 110° C. for 2 h. The reaction was diluted with water (100 mL) and EtOAc (100 mL) and filtered through celite. The aqueous layer was separated and extracted with EtOAc (50 mL×3). The combined organic layers were washed with brine (50 mL), dried over Na₂SO₄, filtered and concentrated. The residue was purified by silica gel column eluted with PE/EtOAc=12/1 to afford methyl 5-methylsulfanylfuran-2-carboxylate (3.5 g) as colorless oil.

### Step 2: Preparation of 5-methylsulfanylfuran-2-carboxylic Acid (Int-35)

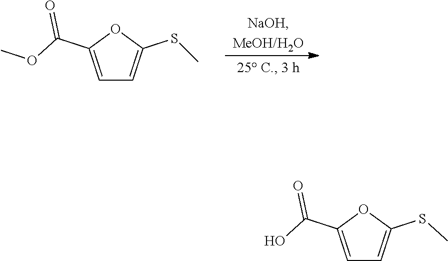

A mixture of methyl 5-methylsulfanylfuran-2-carboxylate (500 mg, 2.90 mmol) and NaOH (929 mg, 23.2 mmol) in MeOH (5 ml) and Water (2 ml) was stirred at room temperature for 3 h. The solution was concentrated in vacuum and the residue was re-dissolved in water (3 ml). The mixture was acidified by 1N HCl to pH=3-4. The precipitate was collected by filtration and washed with water. The filter cake was dried in vacuum to afford 5-methylsulfanylfuran-2-carboxylic acid (232 mg) as a yellow solid.

MS obsd. (ESI⁺) [(M+H)⁺]: 158.9

### Intermediate Int-36

### 5-methylsulfonylfuran-2-carboxylic Acid

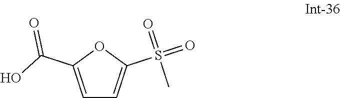

The title compound was prepared according to the following scheme:

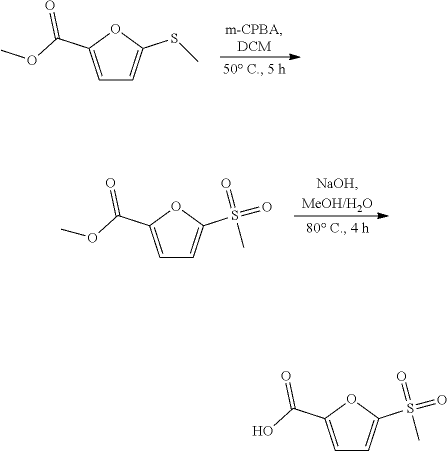

### Step 1: Preparation of methyl 5-methylsulfonylfuran-2-carboxylate

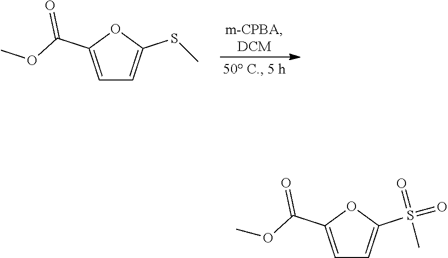

A mixture of methyl 5-methylsulfanylfuran-2-carboxylate (150 mg, 0.87 mmol) and m-CPBA (390 mg, 1.74 mmol) in DCM (5 ml) was stirred at 50° C. for 5 h. The reaction mixture was washed with saturated aqueous Na₂CO₃ (10 ml). The aqueous phase was extracted with DCM (5 ml×3). The organic layers were washed with brine (15 ml), dried over Na₂SO₄ and concentrated in vacuum. The crude product was used for next step without further purification.

### Step 2: Preparation of 5-methylsulfonylfuran-2-carboxylic Acid (Int-36)

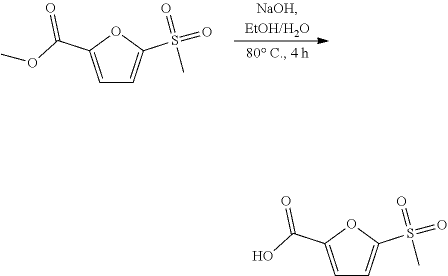

To a mixture of methyl 5-methylsulfonylfuran-2-carboxylate (215 mg, 1.05 mmol) in a mixed solution of EtOH (5 ml) and H₂O (2 ml) was added NaOH (337 mg, 8.42 mmol). Then the reaction mixture was stirred at 80° C. for 4 h. The solution was concentrated in vacuum to remove ethanol. The residue was acidified with 1 N HCl to pH=6-7. The precipitate was collected by filtration and dried in vacuum to afford 5-methylsulfonylfuran-2-carboxylic acid (215 mg) as a light yellow solid.

### Intermediate Int-37

### [5-(2-methoxyethylsulfonyl)furan-2-carbonyl]oxylithium

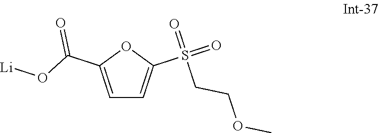

The title compound was prepared according to the following scheme:

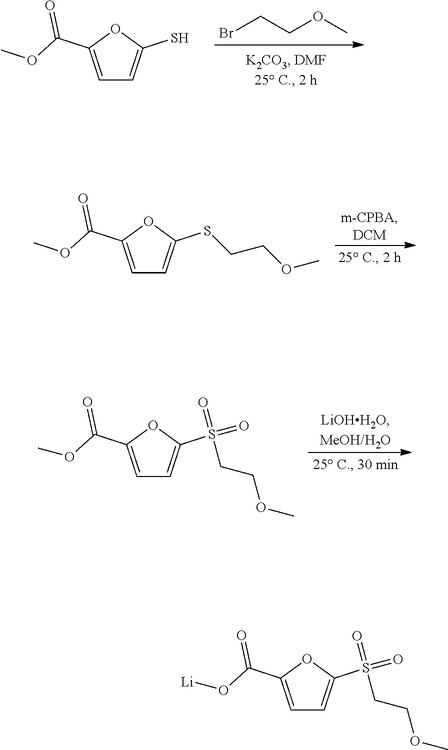

### Step 1: Preparation of methyl 5-(2-methoxyethylsulfanyl)furan-2-carboxylate

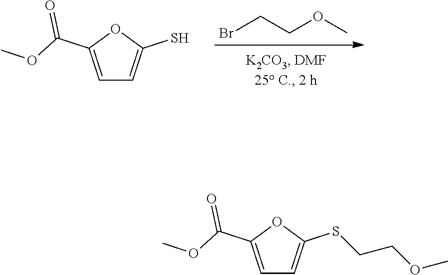

To a mixture of methyl 5-sulfanylfuran-2-carboxylate (300.0 mg, 1.9 mmol, 1 eq) in DMF (3 mL) was added K₂CO₃ (786 mg, 5.69 mmol) and 2-bromoethyl methyl ether (0.71 mL, 7.59 mmol). The reaction was stirred at 25° C. for 2 h. The mixture was diluted with DCM (30 mL×3) and water (20 mL×2). The organic layers were dried over Na₂SO₄ and concentrated. The residue was purified by prep-TLC (PE/EtOAc=19/1) to afford methyl 5-(2-methoxyethylsulfanyl)furan-2-carboxylate (40 mg) as light yellow oil.

MS obsd. (ESI⁺) [(M+H)⁺]: 217.1

### Step 2: Preparation of methyl 5-(2-methoxyethylsulfonyl)furan-2-carboxylate

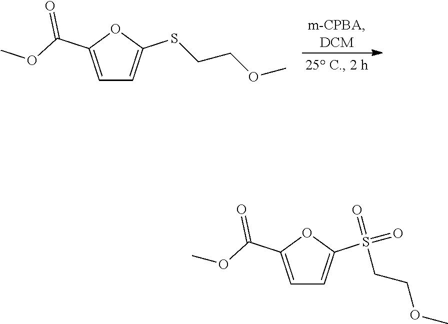

To a solution of methyl 5-(2-methoxyethylsulfanyl)furan-2-carboxylate (10.0 mg, 0.050 mmol) in DCM (2 mL) was added m-CPBA (16 mg, 0.090 mmol). The mixture was stirred at 25° C. for 2 h. The mixture was diluted with DCM (10 mL×3) and water (10 mL×2). The organic layers were dried over Na₂SO₄ and concentrated. The residue was purified by prep-TLC (PE/EtOAc=9/1) to afford methyl 5-(2-methoxyethylsulfonyl)furan-2-carboxylate (7 mg, 0.030 mmol) as light yellow oil.

MS obsd. (ESI⁺) [(M+H)⁺]: 249.1

### Step 3: Preparation of [5-(2-methoxyethylsulfonyl)furan-2-carbonyl]oxylithium (Int-37)

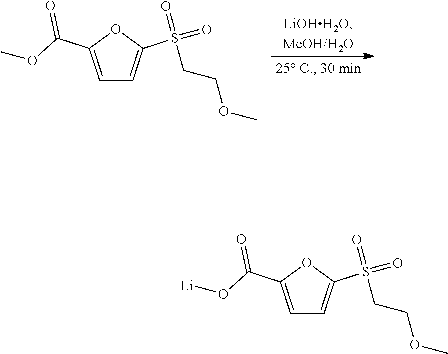

To a solution of methyl 5-(2-methoxyethylsulfonyl)furan-2-carboxylate (150.0 mg, 0.60 mmol) in a mixed solution of MeOH (12 mL) and H₂O (4 mL) was added lithium hydroxide (0.04 mL, 2.42 mmol). The mixture was stirred at 25° C. for 30 min. The reaction mixture was concentrated to remove MeOH. The aqueous layer was lyophilized to afford [5-(2-methoxyethylsulfonyl)furan-2-carbonyl]oxylithium (130 mg) as a yellow solid.

MS obsd. (ESI⁺) [(M+H)⁺]: 235.1

### Intermediate Int-38

### 5-(methylsulfanylmethyl)furan-2-carboxylic Acid

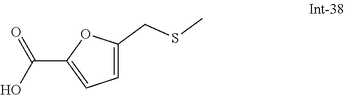

The title compound was prepared in analogy to the procedure described for the preparation of 5-methylsulfanylfuran-2-carboxylic acid (Int-35), by using ethyl 5-(chloromethyl)furan-2-carboxylate instead of methyl 5-bromofuran-2-carboxylate.

### Intermediate Int-39

### 5-(1-methyl-1-methylsulfinyl-ethyl)furan-2-carboxylic Acid

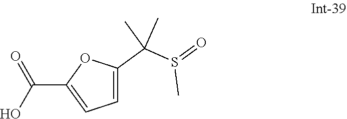

The title compound was prepared according to the following scheme:

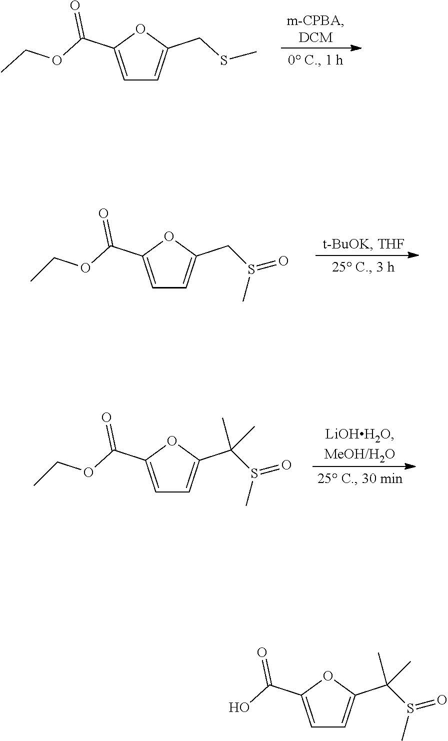 [SMILES: CCOC(=O)c1ccc(CSC)o1 ; CCOC(=O)c1ccc(CS(C)=O)o1 ; CCOC(=O)c1ccc(C(C)(C)S(C)=O)o1 ; CS(=O)C(C)(C)c1ccc(C(=O)O)o1]

### Step 1: Preparation of ethyl 5-(methylsulfinylmethyl)furan-2-carboxylate

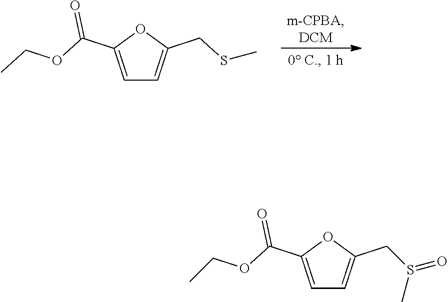

To a solution of ethyl 5-((methylthio)methyl)furan-2-carboxylate (2 g, 9.99 mmol) in DCM (20 ml) was added m-CPBA (2.46 g, 9.99 mmol) portionwise at 0° C. Then the reaction was stirred at the same temperature for 1 h. The reaction solution was washed with saturated aqueous NaHCO₃ (50 ml), dried over Na₂SO₄ and concentrated in vacuum. The residue was purified by silica gel column eluted with DCM/MeOH=40/1 to afford ethyl 5-(methylsulfinylmethyl)furan-2-carboxylate (1.9 g) as colorless oil.

MS obsd. (ESI⁺) [(M+H)⁺]: 217.1

### Step 2: Preparation of ethyl 5-(1-methyl-1-methylsulfinyl-ethyl)furan-2-carboxylate

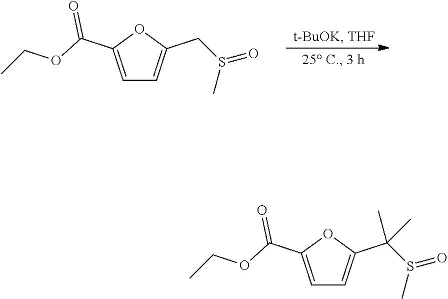 [SMILES: CCOC(=O)c1ccc(CS(C)=O)o1 ; CCOC(=O)c1ccc(C(C)(C)S(C)=O)o1]

To a mixture of ethyl 5-(methylsulfinylmethyl)furan-2-carboxylate (200 mg, 0.93 mmol) in THF (2 ml) was added t-BuOK (259 mg, 2.31 mmol) portionwise. After stirring at 25° C. for 30 min, iodomethane (158 mg, 1.11 mmol) was added. The reaction was stirred at 25° C. for 3 h. The reaction was filtered and washed with DCM (10 mL). The filtrate was concentrated in vacuum and the residue was purified by silica gel column eluted with DCM/MeOH=30/1 to afford ethyl 5-(1-methyl-1-methylsulfinyl-ethyl)furan-2-carboxylate (90 mg) as a white solid.

MS obsd. (ESI⁺) [(M+Na)+]: 267.2

### Step 3: Preparation of 5-(1-methyl-1-methylsulfinyl-ethyl)furan-2-carboxylic Acid (Int-39)

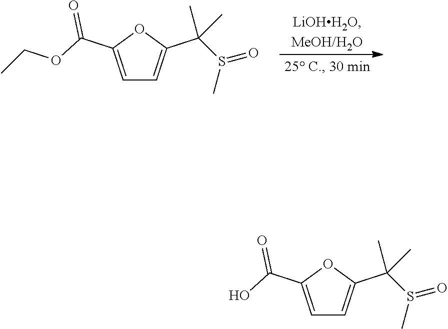 [SMILES: CCOC(=O)c1ccc(C(C)(C)S(C)=O)o1 ; CS(=O)C(C)(C)c1ccc(C(=O)O)o1]

To a mixture of ethyl 5-(1-methyl-1-methylsulfinyl-ethyl)furan-2-carboxylate (80 mg, 0.327 mmol) in MeOH (2 ml) and Water (0.5 ml) was added lithium hydroxide hydrate (55 mg, 1.31 mmol). Then the reaction was stirred at 25° C. for 30 min. The reaction mixture was concentrated in vacuum. The residue was acidified by 1N HCl to pH=3-4. The resulting mixture was concentrated in vacuum to afford 5-(1-methyl-1-methylsulfinyl-ethyl)furan-2-carboxylic acid (140 mg) as a yellow solid. The crude was used for next step without further purification.

MS obsd. (ESI⁺) [(M+H)⁺]: 217.0

### Intermediate Int-40

### 2-cyclopropylsulfonylpyridine-4-carboxylic Acid

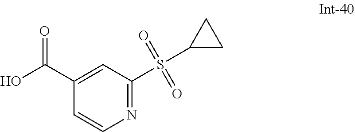 [SMILES: O=C(O)c1ccnc(S(=O)(=O)C2CC2)c1]

The title compound was prepared in analogy to the procedure described for the preparation of 5-cyclopropylsulfonylfuran-2-carboxylic acid (Int-12), by using 2-bromoisonicotinic acid instead of 5-bromo-2-furoic acid.

### Intermediate Int-41

### 2-isopropylsulfonylpyridine-4-carboxylic Acid

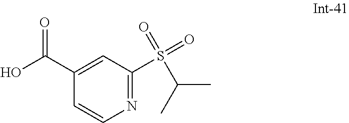

The title compound was prepared according to the following scheme:

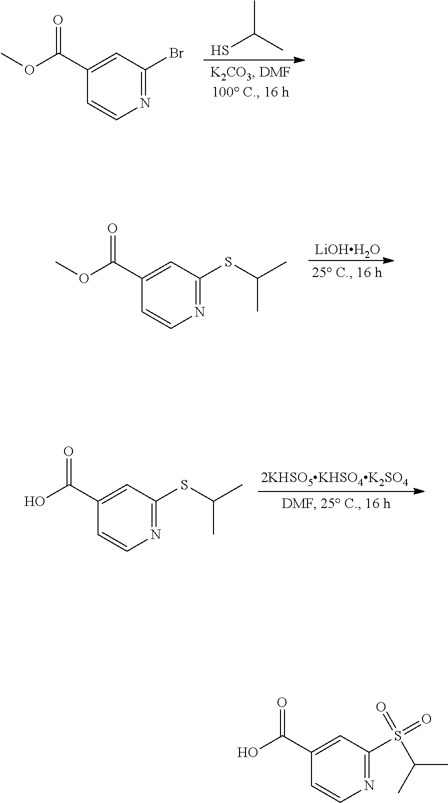

### Step 1: Preparation of methyl 2-isopropylsulfanylpyridine-4-carboxylate

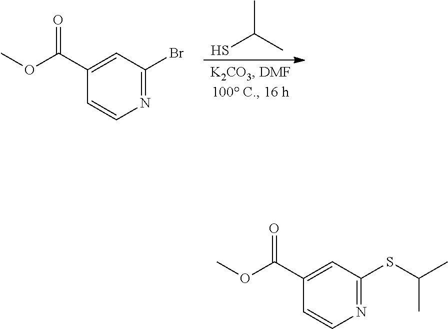

To a solution of methyl 2-bromoisonicotinate (2 g, 9.26 mmol) in DMF (19 mL) was added dipotassium carbonate (1.54 g, 11.1 mmol) and 2-propanethiol (1.41 g, 18.5 mmol). The reaction mixture was irradiated in a microwave reactor at 100° C. for 16 h. The reaction mixture was filtered and the cake was washed with DCM (20 mL). The filtrate was concentrated and the residue was purified by silica gel column eluted with DCM/MeOH=10/1 to 5/1 to afford methyl 2-isopropylsulfanylpyridine-4-carboxylate (1.8 g) as light yellow oil.

MS obsd. (ESI⁺) [(M+H)⁺]: 212.1

### Step 2: Preparation of 2-isopropylsulfanylpyridine-4-carboxylic Acid

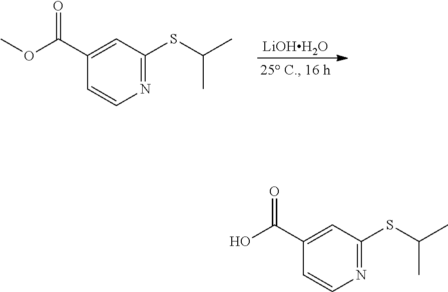

To a solution of methyl 2-isopropylsulfanylpyridine-4-carboxylate (1.6 g, 7.57 mmol) in methanol (32 mL) and was added LiOH·H₂O (0.65 g, 27.2 mmol). The reaction was stirred at 25° C. for 16 h. The mixture was poured into water (20 mL), concentrated in vacuum to remove most of MeOH and adjusted to pH=3 with 31% HCl, a white solid was formed. The mixture was filtered to give 2-isopropylsulfanylpyridine-4-carboxylic acid (1.4 g) as a white solid.

MS obsd. (ESI⁺) [(M+H)⁺]: 198.2

### Step 3: Preparation of 2-isopropylsulfonylpyridine-4-carboxylic Acid

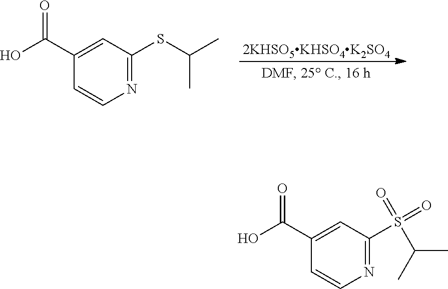

To a solution of 2-isopropylsulfanylpyridine-4-carboxylic acid (1.4 g, 7.1 mmol) in DMF (10 mL) and THF (50 mL) was added 2KHSO₅·KHSO₄·K₂SO₄ (1.57 g, 25.5 mmol). The reaction mixture was stirred at 25° C. for 16 h. The mixture was filtered and the filtrate was concentrated.

The residue was purified by prep-HPLC to give 2-isopropylsulfonylpyridine-4-carboxylic acid (1.6 g) as a light yellow solid.

MS obsd. (ESI⁺) [(M+H)⁺]: 230.2

### Intermediate Int-42

### 2-cyclopropylsulfonylpyridine-4-carboxylic Acid

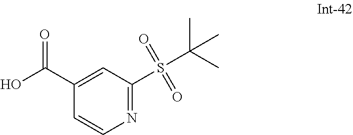 [SMILES: CC(C)(C)S(=O)(=O)c1cc(C(=O)O)ccn1]

The title compound was prepared in analogy to the procedure described for the preparation of 2-isopropylsulfonylpyridine-4-carboxylic acid (Int-41), by using 2-methyl-2-propanethiol instead of 2-propanethiol.

### Intermediate Int-43

### 2-(1-methylsulfonylethyl)pyridine-4-carboxylic Acid

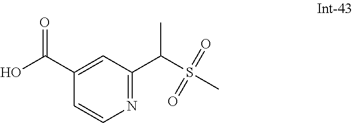

The title compound was prepared according to the following scheme:

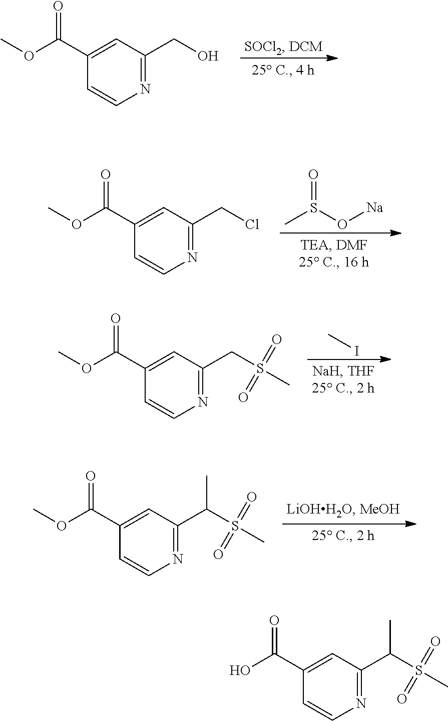

### Step 1: Preparation of methyl 2-(chloromethyl)pyridine-4-carboxylate

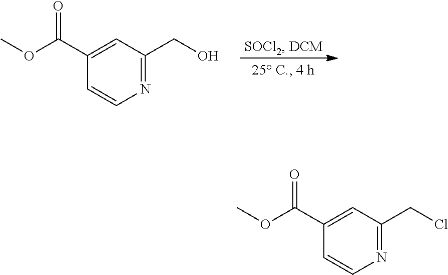

To a solution of methyl 2-(hydroxymethyl)pyridine-4-carboxylate (10.0 g, 59.8 mmol) in DCM (250 mL) was added thionyl chloride (8.68 mL, 119.7 mmol). The reaction mixture was stirred at 25° C. for 4 h. The mixture was concentrated in vacuum to afford methyl 2-(chloromethyl)pyridine-4-carboxylate (11.1 g) as a light yellow solid.

MS obsd. (ESI⁺) [(M+H)⁺]: 186.0

### Step 2: Preparation of methyl 2-(methylsulfonylmethyl)pyridine-4-carboxylate

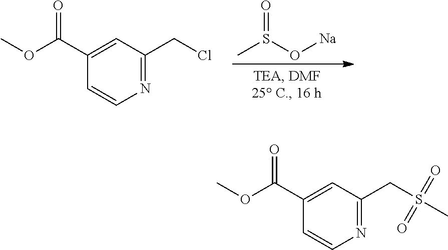

To a solution of methylsulfinyloxysodium (9.2 g, 89.7 mmol) in DMF (250 mL) was added methyl 2-(chloromethyl)pyridine-4-carboxylate (11.1 g, 59.8 mmol) and TEA (16.7 ml, 119.7 mmol). The reaction mixture was stirred for 16 h at 25° C. The reaction mixture was concentrated to dryness and the residue was diluted with EtOAc (80 mL). 1 N HCl was added to adjust pH=2-3. The separated organic phase was washed with brine (80 mL), dried over Na₂SO₄ and concentrated in vacuum. The residue was purified by silica gel column eluted with PE/EtOAc=1/1 to give methyl 2-(methylsulfonylmethyl)pyridine-4-carboxylate (6.0 g) as a light yellow solid.

MS obsd. (ESI⁺) [(M+H)⁺]: 230.1

### Step 3: Preparation of methyl 2-(1-methylsulfonylethyl)pyridine-4-carboxylate

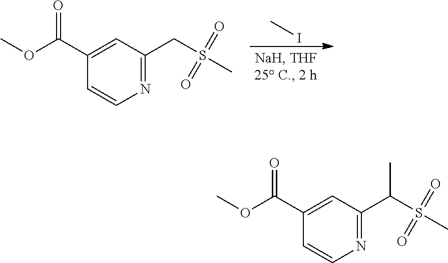

To a solution of methyl 2-(methylsulfonylmethyl)pyridine-4-carboxylate (4.7 g, 20.5 mmol) in THF (150 mL) was added sodium hydride (820 mg, 20.5 mmol). The reaction was stirred for 30 min, then iodomethane (2.55 mL, 41 mmol) was added and stirred for 2 h at 25° C. The reaction mixture was quenched with MeOH (15 mL), concentrated and purified by silica gel column eluted with PE/EtOAc=4/1 to give the product methyl 2-(1-methylsulfonylethyl)pyridine-4-carboxylate (2.18 g) as a light yellow solid.

MS obsd. (ESI⁺) [(M+H)⁺]: 244.1

### Step 4: Preparation of 2-(1-methylsulfonylcyclopropyl)thiazole-5-carboxylic Acid (Int-43)

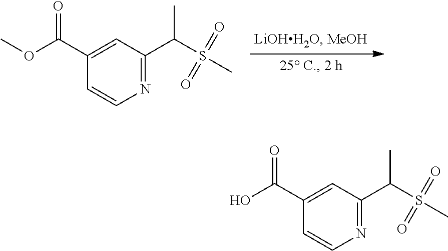

To a solution of methyl 2-(1-methylsulfonylethyl)pyridine-4-carboxylate (760 mg, 3.12 mmol) in MeOH (20 mL) was added LiOH·H₂O (300 mg, 12.5 mmol). The reaction mixture was stirred at 25° C. for 2 h. The mixture was concentrated and purified by prep-HPLC to afford 2-(1-methylsulfonylethyl)pyridine-4-carboxylic acid (523 mg) (Int-43) as a white solid.

MS obsd. (ESI⁺) [(M+H)⁺]: 230.1

### Intermediate Int-44

### 2-(1-methyl-1-methylsulfonyl-ethyl)pyridine-4-carboxylic Acid

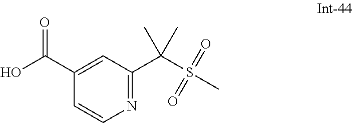 [SMILES: CC(C)(c1cc(C(=O)O)ccn1)S(C)(=O)=O]

The title compound was prepared in analogy to the procedure described for the preparation of 2-isopropylsulfonylpyridine-4-carboxylic acid (Int-43), by extending the reaction time from 2 h to 16 h of step 3.

### Intermediate Int-45

### 3-(1-methylsulfonylethyl)-1H-pyrazole

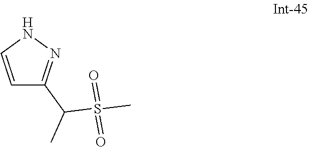

The title compound was prepared according to the following scheme:

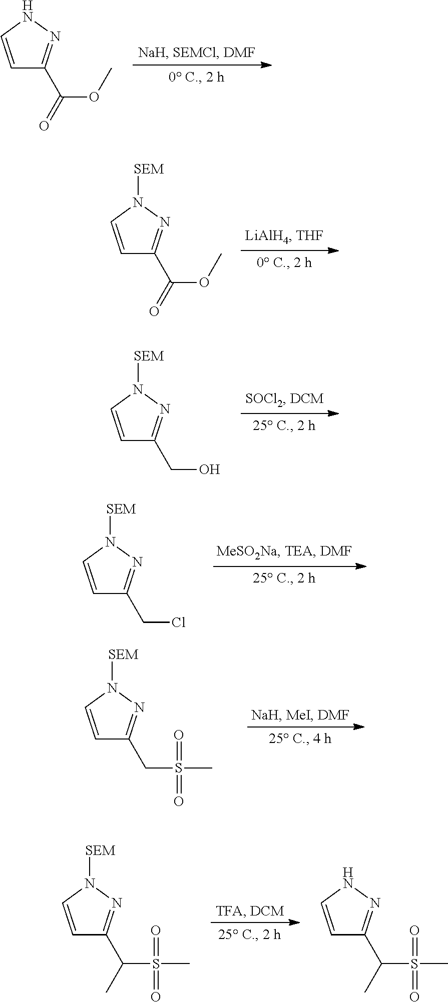

### Step 1: Preparation of methyl 1-(2-trimethylsilylethoxymethyl)pyrazole-3-carboxylate

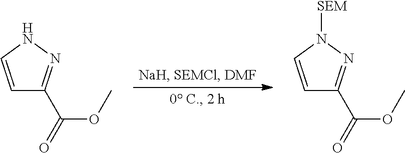

To a solution of methyl 1H-pyrazole-3-carboxylate (20.0 g, 159 mmol) in DMF (200 mL) was added sodium hydride (4.57 g, 190 mmol, 60% in oil) at 0° C. After stirring at 0° C. for 30 min, SEMCl (31.7 g, 190 mmol) was added portionwise at 0° C. Then the reaction mixture was stirred at 0° C. for another 2 h. The reaction was quenched with water (10 mL). The resulting solution was diluted with EtOAc (500 mL), washed with water (800 mL×2) and brine (1000 mL). The organic layer was dried over Na₂SO₄ and concentrated in vacuum. The residue was purified by silica gel column eluted with PE/EtOAc=4/1 to give methyl 1-(2-trimethylsilylethoxymethyl) pyrazole-3-carboxylate (40 g) as light yellow oil

MS obsd. (ESI⁺) [(M+H)⁺]: 257.2.

### Step 2: Preparation of [1-(2-trimethylsilylethoxymethyl)pyrazol-3-yl]methanol

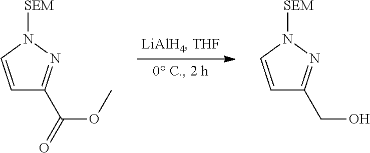 [SMILES: COC(=O)c1ccn(COCC[Si](C)(C)C)n1 ; C[Si](C)(C)CCOCn1ccc(CO)n1]

To a solution of methyl 1-(2-trimethylsilylethoxymethyl) pyrazole-3-carboxylate (3.0 g, 11.1 mmol) in THF (30 mL) was added LiAlH₄ (0.51 g, 13.3 mmol) at 0° C. The reaction mixture was stirred at 0° C. for 2 h. The reaction was quenched with H₂O (1 mL). After filtered through celite, the filtrate was concentrated in vacuum to give [1-(2-trimethylsilylethoxymethyl)pyrazol-3-yl]methanol (1.7 g) as light yellow oil. The crude was used directly in next step.

MS obsd. (ESI⁺) [(M+H)⁺]: 229.2.

### Step 3: Preparation of 2-[[3-(chloromethyl)pyrazol-1-yl]methoxy]ethyl-trimethyl-silane

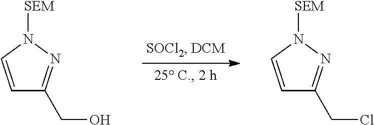 [SMILES: C[Si](C)(C)CCOCn1ccc(CO)n1 ; C[Si](C)(C)CCOCn1ccc(CCl)n1]

To a solution of [1-(2-trimethylsilylethoxymethyl)pyrazol-3-yl]methanol (1.7 g, 7.44 mmol) in DCM (20 mL) was added SOCl₂ (0.81 mL, 11.2 mmol) slowly. The mixture was stirred at 25° C. for 2 h and then concentrated in vacuum to give 2-[[3-(chloromethyl)pyrazol-1-yl]methoxy]ethyl-trimethyl-silane (1.85 g) as light yellow oil.

MS obsd. (ESI⁺) [(M+H)⁺]: 247.2.

### Step 4: Preparation of trimethyl-[2-[[3-(methylsulfonylmethyl)pyrazol-1-yl]methoxy]ethyl]silane

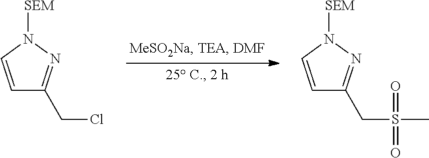 [SMILES: C[Si](C)(C)CCOCn1ccc(CCl)n1 ; C[Si](C)(C)CCOCn1ccc(CS(C)(=O)=O)n1]

A mixture of 2-[[3-(chloromethyl)pyrazol-1-yl]methoxy]ethyl-trimethyl-silane (1.85 g, 7.5 mmol), sodium methanesulfinate (1.15 g, 11.2 mmol) and TEA (3.1 mL, 22.5 mmol) in DMF (15 mL) was stirred at 25° C. for 2 h. After filtration, the filtrate was purified by reversed flash column (eluting with ACN/H₂O=50/50) to give trimethyl-[2-[[3-(methylsulfonylmethyl)pyrazol-1-yl]methoxy]ethyl]silane (600 mg) as a light yellow solid.

MS obsd. (ESI⁺) [(M+H)⁺]: 313.1.

### Step 5: Preparation of trimethyl-[2-[[3-(1-methylsulfonylethyl)pyrazol-1-yl]methoxy]ethyl]silane

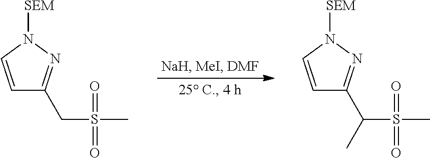 [SMILES: C[Si](C)(C)CCOCn1ccc(CS(C)(=O)=O)n1 ; CC(c1ccn(COCC[Si](C)(C)C)n1)S(C)(=O)=O]

To a solution of trimethyl-[2-[[3-(methylsulfonylmethyl)pyrazol-1-yl]methoxy]ethyl]silane (150 mg, 0.52 mmol) in DMF (5 mL) was added sodium hydride (25 mg, 0.62 mmol). After stirring at 25° C. for 1 h, iodomethane (88 mg, 0.62 mmol) was added. The reaction mixture was stirred for another 3 h. The resulting solution was purified by reversed flash column (eluting with acetonitrile/H₂O=50/50) to give trimethyl-[2-[[3-(1-methylsulfonylethyl)pyrazol-1-yl]methoxy]ethyl]silane (100 mg) as light yellow oil.

MS obsd. (ESI⁺) [(M+H)⁺]: 305.2.

### Step 6: Preparation of 3-(1-methylsulfonylethyl)-1H-pyrazole

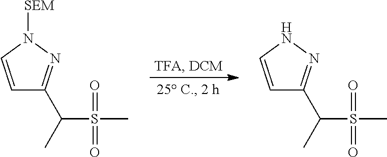 [SMILES: CC(c1ccn(COCC[Si](C)(C)C)n1)S(C)(=O)=O ; CC(c1cc[nH]n1)S(C)(=O)=O]

A mixture of trimethyl-[2-[[3-(1-methylsulfonylethyl)pyrazol-1-yl]methoxy]ethyl]silane (150 mg, 0.49 mmol) and TFA (1.0 mL, 13.5 mmol) in DCM (3 mL) was stirred at 25° C. for 2 h. After filtration, the filtrate was concentrated in vacuum to give 3-(1-methylsulfonylethyl)-JH-pyrazole (85 mg) (Int-45) as light red oil. The crude was used for next step without further purification.

MS obsd. (ESI⁺) [(M+H)⁺]: 175.1.

### Intermediate Int-46

### 3-(methylsulfonylmethyl)-1H-pyrazole

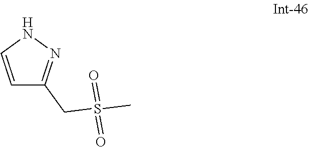

The title compound was prepared according to the following scheme:

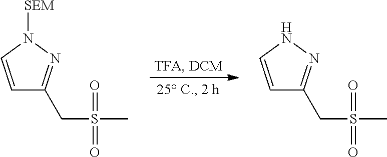

A mixture of trimethyl-[2-[[3-(methylsulfonylmethyl)pyrazol-1-yl]methoxy]ethyl]silane (150 mg, 0.52 mmol) and TFA (1.0 mL, 13.5 mmol) in DCM (3 mL) was stirred at 25° C. for 2 h. After filtration, the filtrate was concentrated in vacuum to give 3-(methylsulfonylmethyl)-JH-pyrazole (80 mg) (Int-46) as light red oil. The crude was used for next step without further purification.

MS obsd. (ESI⁺) [(M+H)⁺]: 161.1.

### Intermediate Int-47

### 3-(methylsulfonylmethyl)-1H-pyrazole

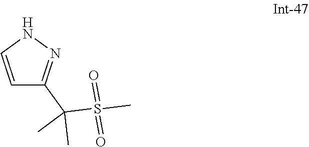

The title compound was prepared in analogy to the procedure described for the preparation of 2-isopropylsulfonylpyridine-4-carboxylic acid (Int-45), by increasing the equivalent of the sodium hydride and iodomethane from 1.2 to 5.

### Intermediate Int-48

### 3-(1-methylsulfonylcyclopropyl)-1H-pyrazole

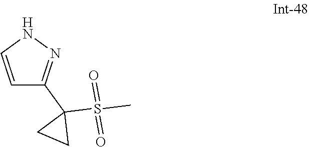

The title compound was prepared according to the following scheme:

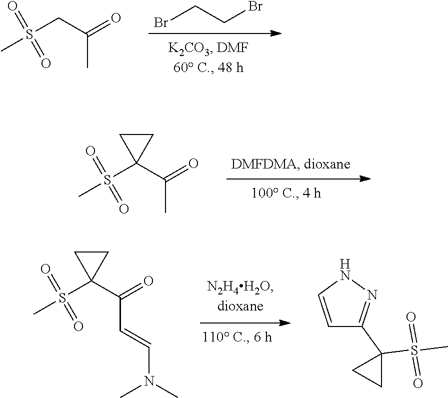

### Step 1: Preparation of 1-(1-methylsulfonylcyclopropyl)ethanone

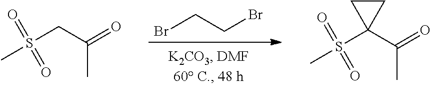

To a solution of methylsulfonylacetone (10.0 g, 73.4 mmol, CAS No: 5000-46-4) and K₂CO₃ (3.04 g, 220.3 mmol) in DMF (120 mL) was added 1,2-dibromoethane (9.49 mL, 110.2 mmol). The resulting mixture was heated to 60° C. and stirred for 48 h. After cooling to ambient temperature, the resulting solution was diluted with water (1000 mL) and extracted with EtOAc (150 mL×5). The combined organic layers were washed with brine (200 mL), dried over Na₂SO₄ and concentrated in vacuum. The residue was purified by silica gel column eluted with PE/EtOAc=3/2 to give 1-(1-methylsulfonylcyclopropyl)ethanone (5.1 g) as a colorless solid.

MS obsd. (ESI⁺) [(M+H)⁺]: 163.1. ¹H NMR (400 MHz, CDCl₃) δ ppm: 3.12 (s, 3H), 2.18 (s, 3H), 1.84-1.88 (m, 2H), 1.59-1.63 (m, 2H).

### Step 2: Preparation of (E)-3-(dimethylamino)-1-(1-methylsulfonylcyclopropyl)prop-2-en-1-one

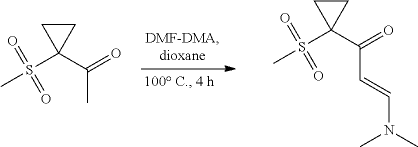

To a solution of 1-(1-methylsulfonylcyclopropyl)ethanone (5.0 g, 30.8 mmol) in 1,4-dioxane (50 mL) was added DMF-DMA (12.3 mL, 92.5 mmol). The resulting mixture was heated to 100° C. and stirred for 4 h. The resulting solution was concentrated in vacuum to give (E)-3-(dimethylamino)-1-(1-methylsulfonylcyclopropyl)prop-2-en-1-one (7.5 g) as yellow liquid. The crude was used for next step without further purification.

MS obsd. (ESI⁺) [(M+H)⁺]: 218.1.

### Step 3: Preparation of 3-(1-methylsulfonylcyclopropyl)-1H-pyrazole

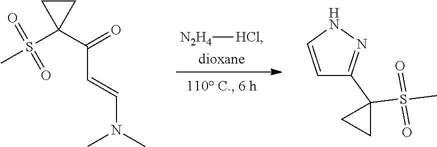

To a solution of (E)-3-(dimethylamino)-1-(1-methylsulfonylcyclopropyl)prop-2-en-1-one (3.29 g, 12.9 mmol) in 1,4-dioxane (50 mL) was added N₂H₄—HCl (0.93 g, 13.5 mmol) under nitrogen atmosphere. The reaction mixture was refluxed for 6 h. The resulting solution was concentrated in vacuum to give 3-(1-methylsulfonylcyclopropyl)-1H-pyrazole (2.8 g) (Int-48) as yellow liquid. The crude was used for next step without further purification.

MS obsd. (ESI⁺) [(M+H)⁺]:187.2.

### Intermediate Int-49

### 5-cyclopropylsulfanylfuran-2-carboxylic Acid

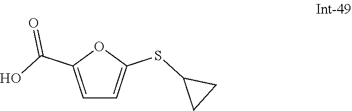 [SMILES: O=C(O)c1ccc(SC2CC2)o1]

The title compound was prepared according to the following scheme:

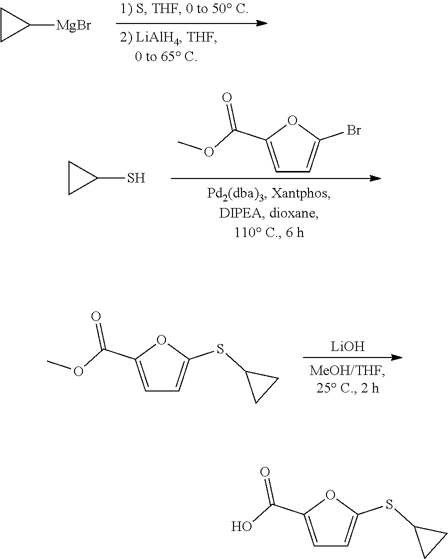

### Step 1: Preparation of cyclopropanethiol

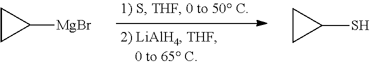

To a solution of cyclopropylmagnesium bromide (10 mL, 5 mmol) in THF was added sulfur (160 mg, 0.63 mmol) at 0° C. Then the solution was heated at 50° C. with stirring for 3 h. After being cooled in an ice-bath, lithium aluminum hydride (5 mL, 5 mmol) in THF was added. The resulting mixture was stirred at 65° C. for 0.5 h and quenched by H₂O (0.5 mL) at 0° C., then acidified by H₂SO₄ (5% v/v, 20 mL). The organic phase containing cyclopropanethiol was separated, dried over anhydrous Na₂SO₄ and used for the next step without purification.

### Step 2: Preparation of methyl 5-cyclopropylsulfanylfuran-2-carboxylate

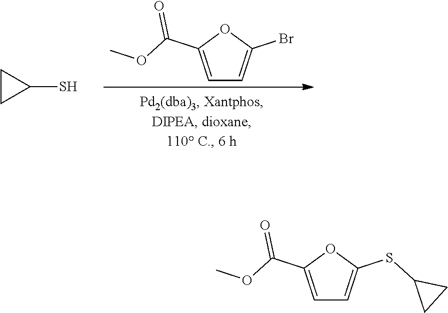

To a mixture of methyl 5-bromo-2-furoate (410 mg, 2 mmol), tris(dibenzylideneacetone)dipalladium (0) (183 mg, 0.2 mmol), 9,9-dimethyl-4,5-bis(diphenylphosphino)xanthene (116 mg, 0.2 mmol) and N,N-diisopropylethylamine (1.74 mL, 10 mmol) in 1,4-dioxane (15 mL) was added the above solution containing cyclopropanethiol. After being stirred at 110° C. for 6 h, the mixture was filtered through a short silica gel column. The filtration was concentrated and purified by silica gel column eluted with PE/EtOAc=99/1 to give methyl 5-cyclopropylsulfanylfuran-2-carboxylate (120 mg) as colorless oil.

MS obsd. (ESI⁺) [(M+H)⁺]: 199.1.

### Step 3: Preparation of 5-cyclopropylsulfanylfuran-2-carboxylic Acid

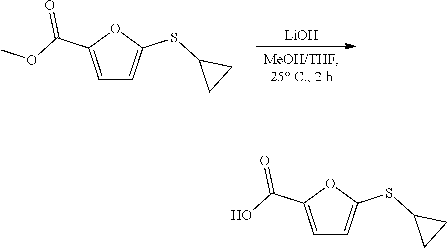

To a solution of methyl 5-cyclopropylsulfanylfuran-2-carboxylate (50 mg, 0.25 mmol) in a mixed solvent of THF (2 mL) and MeOH (2 mL) was added a solution of LiOH in water (1.9 mL, 2 M). After being stirred at 25° C. for 2 h, the mixture was acidified by HCl (1 M) to pH=5, then extracted by EtOAc (10 mL×3). The combined organic layer was dried over Na₂SO₄, filtered and concentrated to give 5-cyclopropylsulfanylfuran-2-carboxylic acid (46 mg) (Int-49) as a white solid.

MS obsd. (ESI⁺) [(M+H)⁺]: 185.1.

### Intermediate Int-50

### 5-(cyclopropylmethylsulfanyl)furan-2-carboxylic Acid

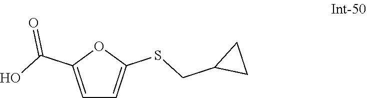 [SMILES: O=C(O)c1ccc(SCC2CC2)o1]

The title compound was prepared according to the following scheme:

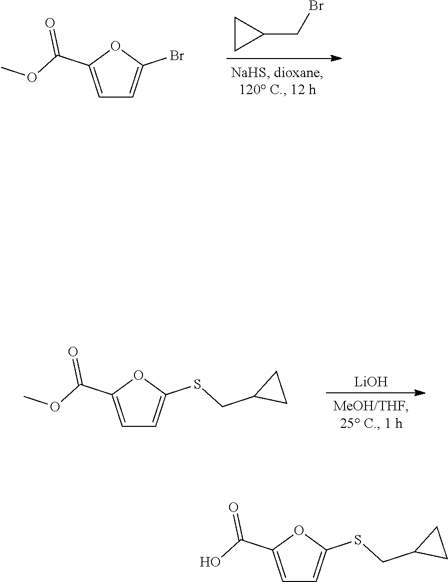

### Step 1: Preparation of methyl 5-(cyclopropylmethylsulfanyl)furan-2-carboxylate

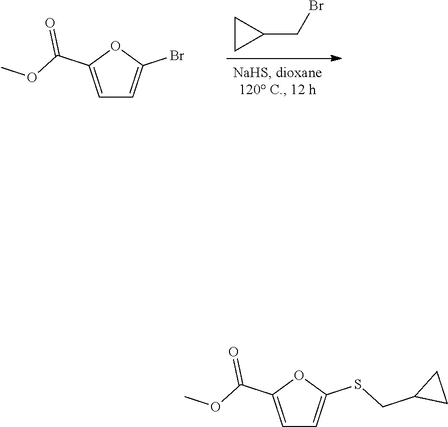

To a solution of methyl 5-bromo-2-furoate (2 g, 9.76 mmol) in 1,4-dioxane (20 mL) was added sodium hydrosulfide (5.5 g, 97.6 mmol) and (bromomethyl)cyclopropane (3.3 mL, 34.1 mmol). After being stirred at 120° C. for 12 h, the reaction mixture was quenched with H₂O (50 mL) and extracted with DCM (50 mL×3). The combined organic layer was washed with brine, dried over Na₂SO₄ and concentrated in vacuo. The residue was purified by flash column (eluting with 100% PE) to afford methyl 5-(cyclopropylmethylsulfanyl)furan-2-carboxylate (580 mg) as colorless oil.

MS obsd. (ESI⁺) [(M+H)⁺]: 213.1. ¹H NMR (400 MHz, CDCl₃) δ ppm: 7.14 (d, J=3.5 Hz, 1H), 6.50 (d, J=3.5 Hz, 1H), 3.89 (s, 3H), 2.86 (d, J=7.2 Hz, 2H), 0.95-1.10 (m, 1H), 0.51-0.61 (m, 2H), 0.14-0.24 (m, 2H).

### Step 2: Preparation of 5-(cyclopropylmethylsulfanyl)furan-2-carboxylic Acid

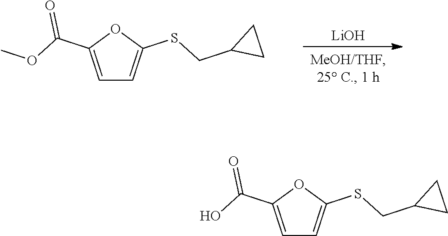

To a solution of methyl 5-(cyclopropylmethylsulfanyl)furan-2-carboxylate (308 mg, 1.45 mmol) in a mixed solvent of THF (2 mL) and methanol (2 mL) was added a solution of LiOH in water (2.2 mL, 2 M). After being stirred at 20° C. for 1 h, the mixture was acidified by HCl (2.5 mL, 2 M). The solvent was evaporated and the residue was separated by EtOAc (20 mL) and water (20 mL). The organic layer was dried over Na₂SO₄, filtered and concentrated to give the 5-(cyclopropylmethylsulfanyl)furan-2-carboxylic acid (280 mg) (Int-50) as a white solid.

MS obsd. (ESI⁺) [(M+H)⁺]: 199.1.

### Intermediate Int-51

### methyl 5-(methylsulfonylmethyl)thiazole-2-carboxylate

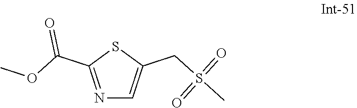

The title compound was prepared in analogy to the procedure described for the preparation of [2-(methylsulfonylmethyl)oxazole-5-carbonyl]oxylithium (Int-18), by using 5-methylthiazole-2-carboxylate instead of ethyl 2-methyloxazole-5-carboxylate.

### Intermediate Int-52

### ethyl 5-(methylsulfonylmethyl)oxazole-2-carboxylate

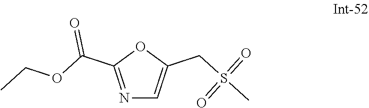

The title compound was prepared in analogy to the procedure described for the preparation of [2-(methylsulfonylmethyl)oxazole-5-carbonyl]oxylithium (Int-18), by using ethyl 5-methyloxazole-2-carboxylate instead of ethyl 2-methyloxazole-5-carboxylate.

### Intermediate Int-53

### Ethyl 3-(1-methylsulfonylcyclopropyl)-1,2,4-oxadiazole-5-carboxylate

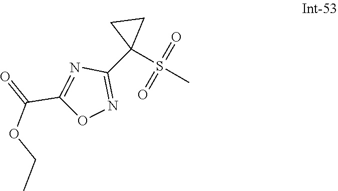 [SMILES: CCOC(=O)c1nc(C2(S(C)(=O)=O)CC2)no1]

The title compound was prepared according to the following scheme:

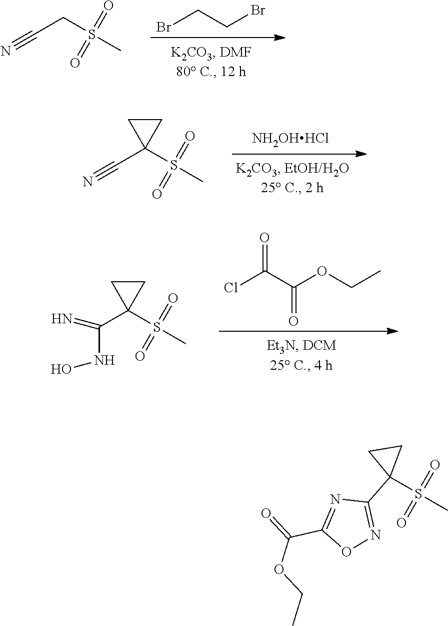

Step 1: Preparation of 1-methylsulfonylcyclopropanecarbonitrile

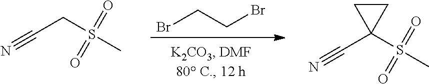

To a mixture of 2-(methylsulfonyl)acetonitrile (5.0 g, 42.0 mmol, CAS No: 2274-42-2) in DMF (50 mL) was added K₂CO₃ (11.6 g, 84.0 mmol) and 1,2-dibromoethane (15.61 g, 84.0 mmol). The mixture was stirred at 80° C. for 12 h. The resulting solution was diluted with water (300 mL) and extracted with EtOAc (80 mL×3). The combined organic layers were washed with brine (20 mL), dried over Na₂SO₄ and concentrated in vacuum to give 1-methylsulfonylcyclopropanecarbonitrile (4.5 g) as a white solid. The crude was used for next step without further purification.

MS obsd. (ESI⁺) [(M+H)⁺]: 146.1.

### Step 2: Preparation of N-hydroxy-1-methylsulfonyl-cyclopropanecarboxamidine

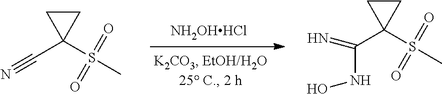

A solution of 1-(methylsulfonyl)cyclopropane-1-carbonitrile (500 mg, 3.45 mmol), hydroxylamine hydrochloride (476 mg, 6.9 mmol) and K₂CO₃ (952 mg, 6.9 mmol) in a mixed solvent of EtOH and H₂O (10 mL, v/v=4/1) was stirred at 25° C. for 2 h. The resulting solution was diluted with water (200 mL) and extracted with EtOAc (40 mL×3). The combined organic layers were washed with brine (30 mL), dried over Nₐ₂SO₄ and concentrated in vacuum to give N-hydroxy-1-methylsulfonyl-cyclopropanecarboxamidine (310 mg) as a white solid. The crude was used for next step without further purification.

MS obsd. (ESI⁺) [(M+H)⁺]: 179.1.

### Step 3: Preparation of ethyl 3-(1-methylsulfonylcyclopropyl)-1,2,4-oxadiazole-5-carboxylate

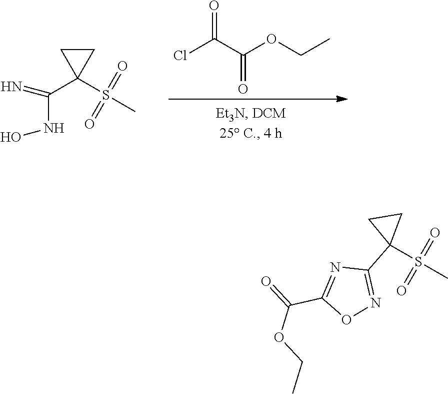

To a mixture of N-hydroxy-1-methylsulfonyl-cyclopropanecarboxamidine (250 mg, 1.4 mmol) in DCM (20 mL) were added Et₃N (283 mg, 2.8 mmol) and ethyl 2-chloro-2-oxoacetate (228 mg, 1.68 mmol). The reaction was stirred at 25° C. for 4 h. The resulting solution was diluted with DCM (100 mL) and washed with water (20 mL×3) and brine (10 mL). The organic layer was dried over Na₂SO₄ and concentrated in vacuum. The residue was purified by silica gel column eluted with PE/EtOAc=1/1 to give ethyl 3-(1-methylsulfonylcyclopropyl)-1,2,4-oxadiazole-5-carboxylate (155 mg) (Int-53) as yellow oil.

MS obsd. (ESI⁺) [(M+H)⁺]: 261.1.

### Intermediate Int-54

### Methyl 5-(1-methylsulfonylcyclopropyl)-1,2,4-oxadiazole-3-carboxylate

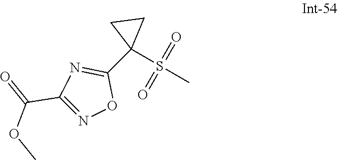 [SMILES: COC(=O)c1noc(C2(S(C)(=O)=O)CC2)n1]

The title compound was prepared according to the following scheme:

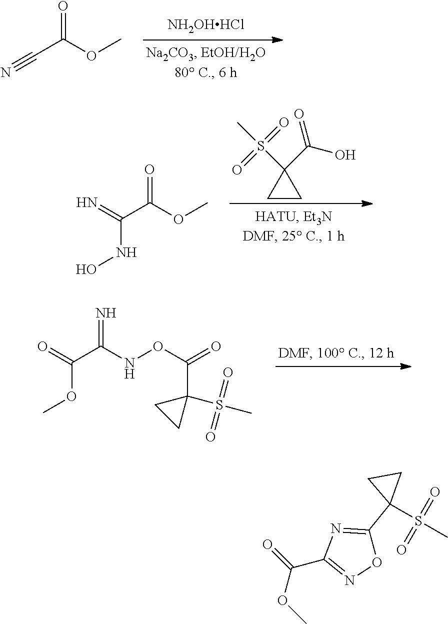

### Step 1: Preparation of methyl 2-(hydroxyamino)-2-imino-acetate

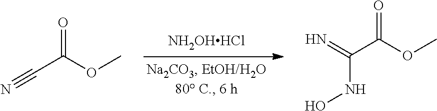

A solution of methyl cyanoformate (8.0 g, 94.1 mmol), sodium carbonate (19.94 g, 188.1 mmol) and NH₂OH·HCl (7.83 mL, 188.1 mmol) in a mixed solution of ethanol and water (250 mL, v/v=4/1) was heated at 80° C. for 6 h. After removed ethanol in vacuum, the resulting solution was diluted with water (100 mL) and extracted with EtOAc (100 mL×3). The combined organic layers were washed with brine (40 mL), dried over Na₂SO₄ and concentrated in vacuum to give methyl 2-(hydroxyamino)-2-imino-acetate (5.4 g) as a light yellow solid.

MS obsd. (ESI⁺) [(M+H)⁺]: 119.1.

### Step 2: Preparation of [(2-methoxy-2-oxo-ethanimidoyl)amino] 1-methylsulfonylcyclopropanecarboxylate

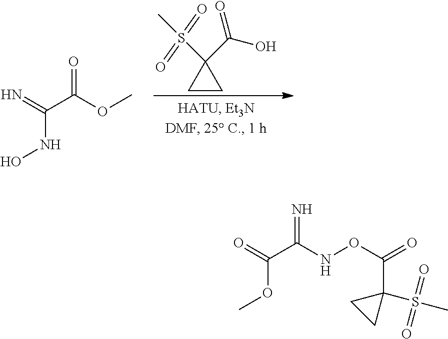

To a solution of 1-methylsulfonylcyclopropanecarboxylic acid (4.17 g, 25.4 mmol) in DMF (40 mL) were added triethylamine (10.6 mL, 76.21 mmol), HATU (10.6 g, 27.94 mmol) and methyl 2-(hydroxyamino)-2-imino-acetate (3.0 g, 25.4 mmol). The reaction mixture was stirred at 25° C. for 1 h. The resulting solution was diluted with H₂O (200 mL) and extracted with EtOAc (10 mL×3). The combined organic layers were washed with brine, dried over Na₂SO₄ and concentrated in vacuum to give [(2-methoxy-2-oxo-ethanimidoyl)amino] 1-methylsulfonylcyclopropanecarboxylate (6 g) as a red solid. The crude product was used for next step without further purification.

MS obsd. (ESI⁺) [(M+H)⁺]: 265.1.

### Step 3: Preparation of methyl 5-(1-methylsulfonylcyclopropyl)-1,2,4-oxadiazole-3-carboxylate

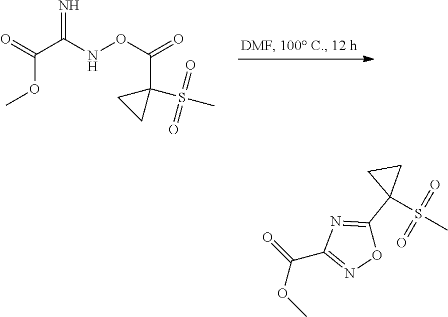 [SMILES: COC(=O)C(=N)NOC(=O)C1(S(C)(=O)=O)CC1 ; "DMF, 100° C., 12 h" ; COC(=O)c1noc(C2(S(C)(=O)=O)CC2)n1]

A solution of [(2-methoxy-2-oxo-ethanimidoyl)amino] 1-methylsulfonylcyclopropanecarboxylate (700 mg, 2.65 mmol) in DMF (10 mL) was heated to 100° C. and stirred for 12 h. After cooled to room temperature, the reaction mixture was filtered and concentrated in vacuum. The residue was purified by prep-HPLC to give methyl 5-(1-methylsulfonylcyclopropyl)-1,2,4-oxadiazole-3-carboxylate (200 mg) (Int-54) as a white solid.

MS obsd. (ESI⁺) [(M+H)⁺]: 247.0.

### Intermediate Int-55

### 5-(1-methylsulfonylethyl)-1-(2-trimethylsilylethoxymethyl)pyrazole-3-carboxylic Acid

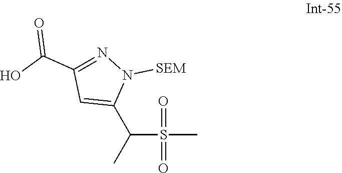 [SMILES: CC(c1cc(C(=O)O)nn1COCC[Si](C)(C)C)S(C)(=O)=O]

The title compound was prepared according to the following scheme:

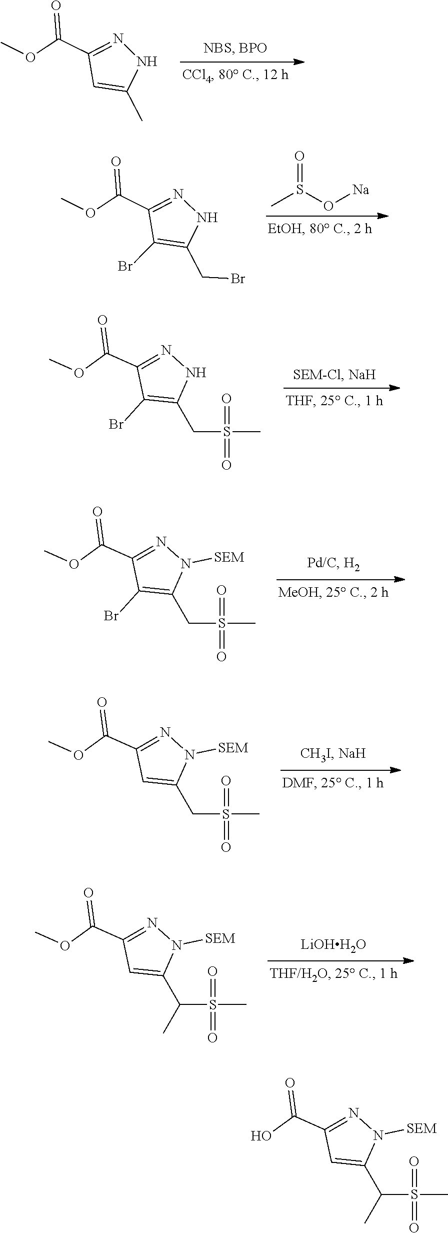

### Step 1: Preparation of methyl 4-bromo-5-(bromomethyl)-1H-pyrazole-3-carboxylate

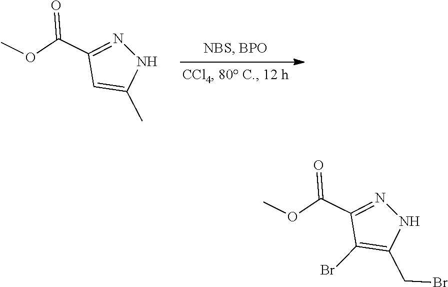

To a solution of methyl 5-methyl-1H-pyrazole-3-carboxylate (4000 mg, 28.54 mmol, CAS No: 25016-17-5) in CCl₄ (50 mL) were added NBS (10161 mg, 57.09 mmol) and BPO (1381 mg, 5.71 mmol). The reaction mixture was stirred for 12 h at 80° C. The resulting mixture was diluted with DCM (300 mL) and washed with water (100 mL×2) and brine (100 mL×2). The organic layer was dried over Na₂SO₄ and concentrated in vacuum. The residue was purified by silica gel column eluted with DCM/MeOH=19/1 to give methyl 4-bromo-5-(bromomethyl)-1H-pyrazole-3-carboxylate (2500 mg) as an off-white solid.

MS obsd. (ESI⁺) [(M+H)⁺]: 299.1.

### Step 2: Preparation of methyl 4-bromo-5-(methylsulfonylmethyl)-1H-pyrazole-3-carboxylate

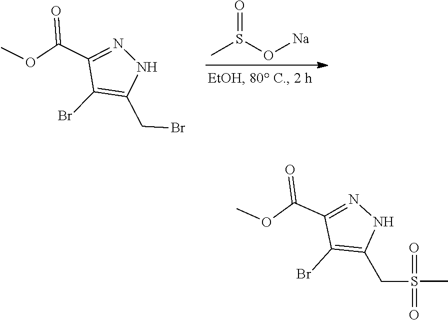

To a solution of methyl 4-bromo-5-(bromomethyl)-1H-pyrazole-3-carboxylate (2000 mg, 6.71 mmol) in ethanol (50 mL) was added sodium methanesulfinate (822 mg, 8.06 mmol). The mixture was stirred at 80° C. for 2 h. After removed ethanol in vacuum, the resulting mixture was diluted with EtOAc (400 mL) and washed with water (100 mL×2) and brine (100 mL×2). The organic layer was dried over Na₂SO₄ and concentrated in vacuum. The residue was purified by silica gel column eluted with DCM/MeOH=19/1 to give methyl 4-bromo-5-(methylsulfonylmethyl)-1H-pyrazole-3-carboxylate (1300 mg) as an off-white solid.

MS obsd. (ESI⁺) [(M+H)⁺]: 297.1.

### Step 3: Preparation of methyl 4-bromo-5-(methylsulfonylmethyl)-1-(2-trimethylsilylethoxymethyl)pyrazole-3-carboxylate

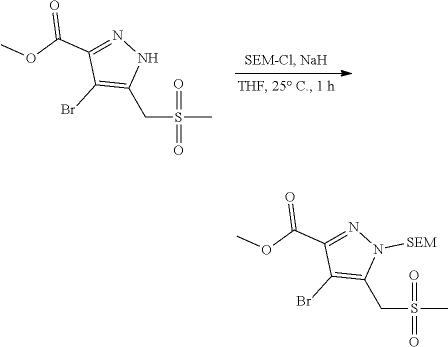

To a solution of methyl 4-bromo-5-(methylsulfonylmethyl)-JH-pyrazole-3-carboxylate (2000 mg, 6.73 mmol) in THF (80 mL) was added sodium hydride (323 mg, 8.08 mmol, 60% in oil) at 0° C. under N₂ atmosphere. After stirred for 0.5 h at 0° C., SEM-Cl (1349 mg, 8.08 mmol) was added. The reaction mixture was warmed to 25° C. and stirred for another 10 h. The resulting solution was diluted with EtOAc (300 mL) and washed with water (100 mL×2) and brine (100 ml×2). The organic layer was dried over Na₂SO₄ and concentrated in vacuum. The residue was purified by silica gel column eluted with DCM/MeOH=19/1 to give methyl 4-bromo-5-(methylsulfonylmethyl)-1-(2-trimethylsilylethoxymethyl)pyrazole-3-carboxylate (1702 mg) as a white solid.

MS obsd. (ESI⁺) [(M+H)⁺]: 427.1.

### Step 4: Preparation of methyl 5-(methylsulfonylmethyl)-1-(2-trimethylsilylethoxymethyl)pyrazole-3-carboxylate

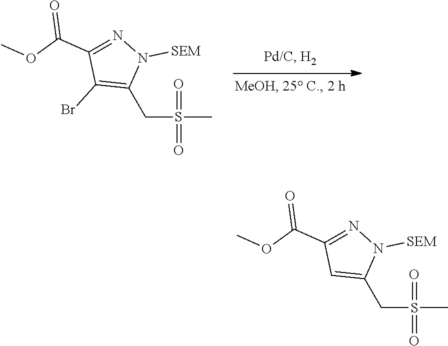

To a solution of methyl 4-bromo-5-(methylsulfonylmethyl)-1-(2-trimethylsilylethoxymethyl)pyrazole-3-carboxylate (1700 mg, 3.98 mmol) in methanol (30 mL) was added Pd/C (170 mg, 10% w/w). The reaction mixture was stirred at room temperature for 2 h under hydrogen atmosphere. After filtered through a celite pad, the filtrate was concentrated in vacuum. The residue was purified by silica gel column eluted with DCM/MeOH=19/1 to give methyl 5-(methylsulfonylmethyl)-1-(2-trimethylsilylethoxymethyl)pyrazole-3-carboxylate (830 mg) as a yellow solid.

MS obsd. (ESI⁺) [(M+H)⁺]: 349.1.

### Step 5: Preparation of methyl 5-(1-methylsulfonylethyl)-1-(2-trimethylsilylethoxymethyl)pyrazole-3-carboxylate

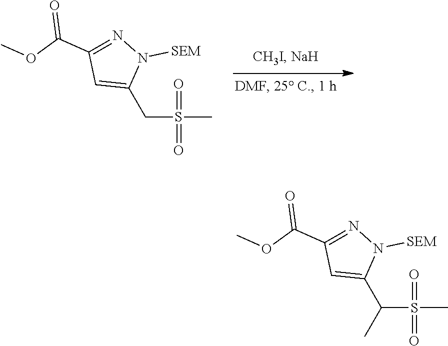

To a solution of methyl 5-(methylsulfonylmethyl)-1-(2-trimethylsilylethoxymethyl)pyrazole-3-carboxylate (100 mg, 0.29 mmol) in DMF (10 mL) was added sodium hydride (28.7 mg, 0.720 mmol, 60% in oil) at 0° C. under nitrogen atmosphere. After stirring for 30 min at 0° C., CH₃I (102 mg, 0.720 mmol) was added. The reaction mixture was warmed to 25° C. and stirred for another 2 h. The resulted mixture was diluted with EtOAc (200 mL) and washed with water (60 mL×2) and brine (100 mL×2). The organic layer was dried over Na₂SO₄ and concentrated in vacuum. The residue was purified by silica gel column eluted with PE/EtOAc=1/1 to give methyl 5-(1-methylsulfonyl-ethyl)-1-(2-trimethylsilylethoxymethyl)pyrazole-3-carboxylate (93 mg) as yellow oil.

MS obsd. (ESI⁺) [(M+H)⁺]: 363.1.

### Step 6: Preparation of 5-(1-methylsulfonylethyl)-1-(2-trimethylsilylethoxymethyl)pyrazole-3-carboxylic Acid

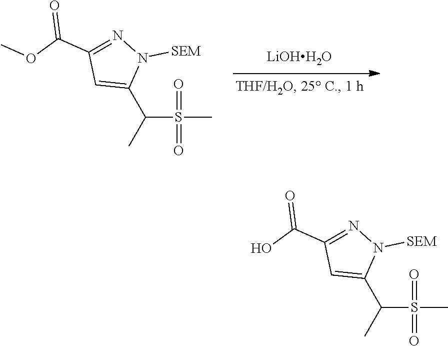

To a solution of methyl 5-(1-methylsulfonyl-ethyl)-1-(2-trimethylsilylethoxymethyl)pyrazole-3-carboxylate (93 mg, 0.250 mmol) in a mixed solution of THF (2 mL) and water (1 mL) was added lithium hydroxide (16 mg, 0.370 mmol). The reaction mixture was stirred at 25° C. for 2 h. The resulting solution was diluted with water (20 mL), adjusted to pH=6 with HCl (2 M) and extracted with EtOAc (20 mL×2). The organic layers were dried over Na₂SO₄ and concentrated in vacuum to give 5-(1-methylsulfonyl-ethyl)-1-(2-trimethylsilylethoxymethyl)pyrazole-3-carboxylic acid (52 mg) (Int-55) as a yellow solid.

MS obsd. (ESI⁺) [(M+H)⁺]: 349.1.

### Example 1

### N-[3-(6-bromo-1,3-benzothiazol-2-yl)-1-bicyclo[1.1.1]pentanyl]-5-(methylsulfonylmethyl)furan-2-carboxamide

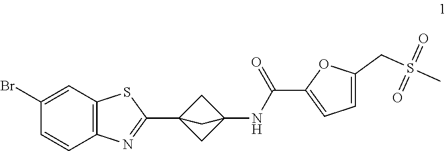 [SMILES: CS(=O)(=O)Cc1ccc(C(=O)NC23CC(c4nc5ccc(Br)cc5s4)(C2)C3)o1]

The title compound was prepared according to the following scheme:

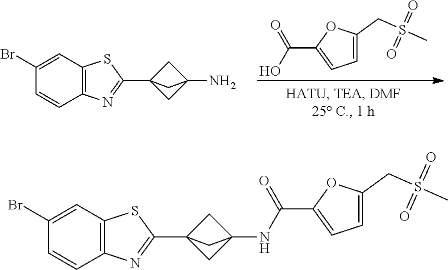 [SMILES: NC12CC(c3nc4ccc(Br)cc4s3)(C1)C2 ; CS(=O)(=O)Cc1ccc(C(=O)O)o1 ; CS(=O)(=O)Cc1ccc(C(=O)NC23CC(c4nc5ccc(Br)cc5s4)(C2)C3)o1]

To a solution of 3-(6-bromo-1,3-benzothiazol-2-yl)bicyclo[1.1.1]pentan-1-amine (Int-1, as the “AMINE” in Table 1) (100 mg, 0.340 mmol), 5-(methylsulfonylmethyl)furan-2-carboxylic acid (69 mg, 0.340 mmol, as the “ACID” in Table 1) in DMF (4 mL) were added HATU (154 mg, 0.410 mmol) and TEA (102 mg, 1.02 mmol). The reaction mixture was stirred at 25° C. for 1 h. The resulting solution was diluted with EtOAc (60 mL) and washed with water (15 mL×2) and brine (15 mL). The organic layer was dried over Na₂SO₄ and concentrated in vacuum. The residue was purified by prep-HPLC (Chromatographic columns: Kromasil-C18 100×21.2 mm 5 um (mobile phase: ACN-H₂O (0.1% FA)) to give N-[3-(6-bromo-1,3-benzothiazol-2-yl)-1-bicyclo[1.1.1]pentanyl]-5-(methylsulfonylmethyl)furan-2-carboxamide (56 mg) as a white solid.

MS obsd. (ESI⁺) [(M+H)⁺]: 480.9. ¹H NMR (400 MHz, CDCl₃) δ ppm: 7.99 (d, J=1.8 Hz, 1H), 7.86 (dd, J=8.8, 1.8 Hz, 1H), 7.55-7.59 (m, 1H), 7.15 (d, J=3.2, 1H), 6.88 (s, 1H), 6.64 (d, J=3.2 Hz, 1H), 4.39 (s, 2H), 2.92 (s, 3H), 2.70 (s, 6H).

The following Example 2 to Example 53 were prepared in analogy to the procedure described for the preparation of Example 1, replacing 3-(6-bromo-1,3-benzothiazol-2-yl)bicyclo[1.1.1]pentan-1-amine (Int-1) with “AMINE”, and replacing 5-(methylsulfonylmethyl)furan-2-carboxylic acid (Int-9) with “ACID”. The “AMINE” and “ACID” are the reagents indicated in Table 1.

TABLE 1
Compounds synthesis and characterization

| Example | Compounds Name and | AMINE and | |
|---|---|---|---|
| No. | Structure | ACID | ¹H NMR (ESI⁺) |
| 2 | 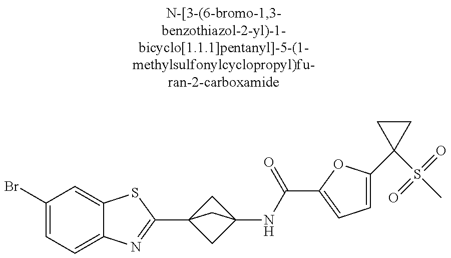 | AMINE: Int-1 ACID: Int-9 | ¹H NMR (400 MHz, CD₃OD) δ ppm: 8.18 (d, J = 2.0 Hz, 1H), 7.84 (d, J = 8.8 Hz, 1H), 7.65 (dd, J = 8.8, 2.0 Hz, 1H), 7.12 (d, J = 3.6 Hz, 1H), 6.78 (d, J = 3.6 Hz, 1H), 3.02 (s, 3H), 2.66 (s, 6H), 1.79 (d, J = 5.2 Hz, 2H), 1.53-1.58 (m, 2H). MS obsd. (ESI⁺) [M + H)⁺]: 503.0 |
| 3 | 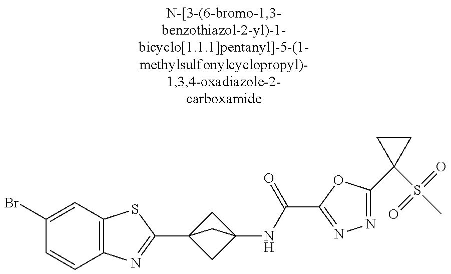 | AMINE: Int-1 ACID: Int-10 | ¹H NMR (400 MHz, DMSO-d₆) δ ppm: 10.16 (s, 1H), 8.40 (d, J = 2.0 Hz, 1H), 7.92 (d, J = 8.8 Hz, 1H), 7.67 (dd, J = 8.8, 2.0 Hz, 1H), 3.31 (s, 3H), 2.59 (s, 6H), 1.84-1.92 (m, 2H), 1.77- 1.84 (m, 2H). MS obsd. (ES⁺) [(M + H)⁺]: 465.3 |
| 4 | 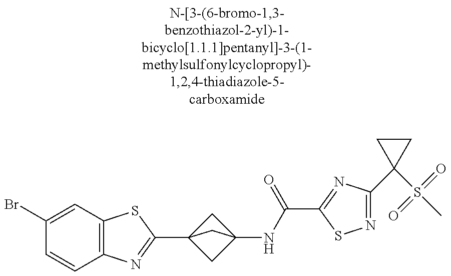 [SMILES: CS(=O)(=O)C1(c2nsc(C(=O)NC34CC(c5nc6ccc(Br)cc6s5)(C3)C4)n2)CC1] | AMINE: Int-1 ACID: Int-11 | ¹H NMR (400 MHz, CDCl₃) d ppm: 8.00 (d, J = 2.0 Hz, 1H), 7.87 (d, J = 8.8 Hz, 1H), 7.56- 7.61 (m, 2H), 3.29 (s, 3H), 2.75 (s, 6H), 2.02-2.06 (m, 2H), 1.76- 1.80 (m, 2H). MS obsd. (ESI+) [(M + H)+]: 524.9 |
| 5 | 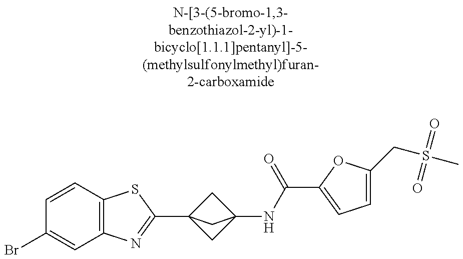 [SMILES: CS(=O)(=O)Cc1ccc(C(=O)NC23CC(c4nc5cc(Br)ccc5s4)(C2)C3)o1] | AMINE: Int-2 ACID: 5- (methylsulfonyl- methyl)furan- 2-carboxylic acid | ¹H NMR (400 MHz, CDCl3) d ppm: 8.18 (s, 1 H), 7.71 (d, J = 8.4 Hz, 1 H), 7.50 (d, J = 8.4 Hz, 1 H), 7.15 (d, J = 3.2 Hz, 1 H), 6.85 (s, 1 H), 6.64 (d, J = 3.2 Hz, 1 H), 4.37 (s, 2 H), 2.92 (s, 3 H), 2.72 (s, 6 H) MS obsd. (ESI+) [(M + H)+]: 480.9 |
| 6 | 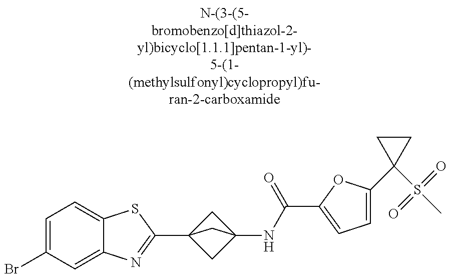 [SMILES: CS(=O)(=O)C1(c2ccc(C(=O)NC34CC(c5nc6cc(Br)ccc6s5)(C3)C4)o2)CC1] | AMINE: Int-2 ACID: Int-9 | ¹H NMR (400 MHz, CD3OD) δ ppm: 8.10 (d, J = 1.8 Hz, 1H), 7.90 (d, J = 8.6 Hz, 1H), 7.56 (dd, J = 8.6, 1.8 Hz, 1H), 7.13 (d, J = 3.6 Hz, 1H), 6.79 (d, J = 3.6 Hz, 1H), 3.02 (s, 3H), 2.66 (s, 6H), 1.77-1.82 (m, 2H), 1.54- 1.58 (m, 2H). MS obsd. (ESI+) [(M + H)+]: 507.0 |
| 7 | 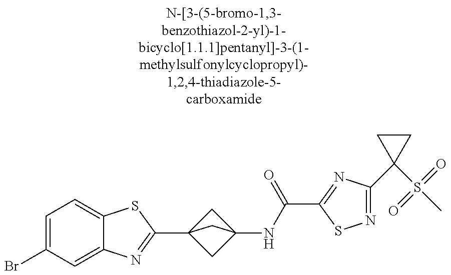 [SMILES: CS(=O)(=O)C1(c2nsc(C(=O)NC34CC(c5nc6cc(Br)ccc6s5)(C3)C4)n2)CC1] | AMINE: Int-2 ACID: Int-11 | ¹H NMR (400 MHz, CDCl3) δ ppm: 8.17 (d, J = 1.6 Hz, 1H), 7.72 (d, J = 8.6 Hz, 1H), 7.63 (s, 1H), 7.50 (dd, J = 8.6, 1.6 Hz, 1H), 3.29 (s, 3H), 2.75 (s, 6H), 2.04 (q, J = 4.8 Hz, 2H), 1.79 (q, J = 4.8 Hz, 2H). MS obsd. (ESI+) [(M + H)+]: 525.0 |
| 8 | 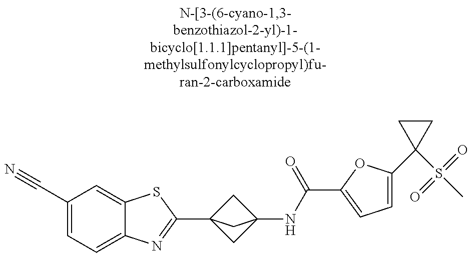 [SMILES: CS(=O)(=O)C1(c2ccc(C(=O)NC34CC(c5nc6ccc(C#N)cc6s5)(C3)C4)o2)CC1] | AMINE: Int-3 ACID: Int-9 | ¹H NMR (400 MHz, CDCl3) δ ppm: 8.20 (d, J = 1.6 Hz, 1H), 8.08 (d, J = 8.6 Hz, 1H), 7.73 (dd, J = 8.6, 1.6 Hz, 1H), 7.12 (d, J = 3.6 Hz, 1H), 6.65 (d, J = 3.6 Hz, 1H), 2.93 (s, 3H), 2.74 (s, 6H), 1.89 (q, J = 4.8 Hz, 2H), 1.46 (q, J = 4.8 Hz, 2H). MS obsd. (ESI+) [(M + H)+]: 454.0 |
| 9 | 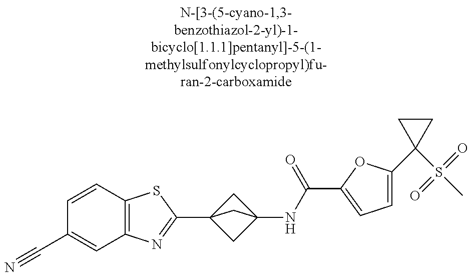 [SMILES: CS(=O)(=O)C1(c2ccc(C(=O)NC34CC(c5nc6cc(C#N)ccc6s5)(C3)C4)o2)CC1] | AMINE: Int-4 ACID: Int-9 | ¹H NMR (400 MHz, DMSO-d6) δ ppm: 9.15 (s, 1H), 8.55 (s, 1H), 8.34 (d, J = 8.4 Hz, 1H), 7.85 (d, J = 8.4 Hz, 1H), 7.14 (d, J = 3.6 Hz, 1H), 6.80 (d, J = 3.6 Hz, 1H), 3.10 (s, 3H), 2.60 (s, 6H), 1.80 (d, J = 5.0 Hz, 2H), 1.57 (d, J = 5.0 Hz, 2H). MS obsd. (ESI+) [(M + H)+]: 454.0 |
| 10 | 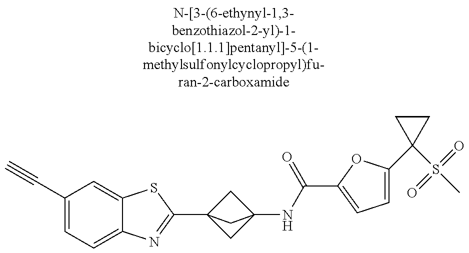 [SMILES: C#Cc1ccc2nc(C34CC(NC(=O)c5ccc(C6(S(C)(=O)=O)CC6)o5)(C3)C4)sc2c1] | AMINE: Int-5 ACID: Int-9 | ¹H NMR (400 MHz, CDCl3) δ ppm: 8.00 (s, 1H), 7.94 (d, J = 8.4 Hz, 1H), 7.58 (dd, J = 8.4 Hz, 1H), 7.11 (d, J = 3.4 Hz, 1H), 6.85 (s, 1H), 6.65 (d, J = 3.4 Hz, 1H), 3.15 (s, 1H), 2.93 (s, 3H), 2.72 (s, 6H), 1.89 (q, J = 5.2 Hz, 2H), 1.46 (q, J = 5.2 Hz, 2H). MS obsd. (ESI+) [(M + H)+]: 452.0 |
| 11 | 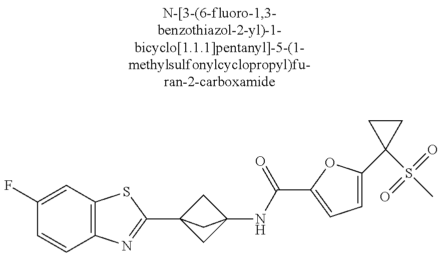 [SMILES: CS(=O)(=O)C1(c2ccc(C(=O)NC34CC(c5nc6ccc(F)cc6s5)(C3)C4)o2)CC1] | AMINE: Int-6 ACID: Int-9 | ¹H NMR (400 MHz, CD3OD) δ ppm: 7.91-7.95 (m, 1H), 7.72- 7.76 (m, 1H), 7.28-7.32 (m, 1H), 7.12 (d, J = 3.6 Hz, 1H), 6.79 (d, J = 3.6 Hz, 1H), 3.02 (s, 3H), 2.65 (s, 6H), 1.80 (d, J = 5.0 Hz, 2H), 1.57 (d, J = 5.0 Hz, 2H). MS obsd. (ESI+) [(M + H)+]: 447.1 |
| 12 | 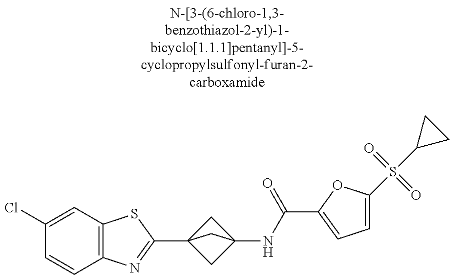 [SMILES: O=C(NC12CC(c3nc4ccc(Cl)cc4s3)(C1)C2)c1ccc(S(=O)(=O)C2CC2)o1] | AMINE: Int-7 ACID: Int-12 | ¹H NMR (400 MHz, CD3OD) δ ppm: 8.04 (d, J = 2.0 Hz, 1H), 7.91 (d, J = 8.8 Hz, 1H), 7.53 (s, 1H), 7.24-7.27 (m, 2H), 2.81- 2.87 (m, 1H), 2.67 (s, 6H), 1.32- 1.36 (m, 2H), 1.12-1.18 (m, 2H). MS obsd. (ESI+) [(M + H)+]: 449.1 |
| 13 | 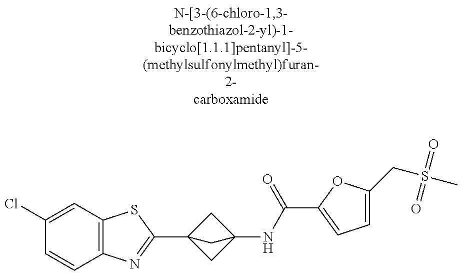 [SMILES: CS(=O)(=O)Cc1ccc(C(=O)NC23CC(c4nc5ccc(Cl)cc5s4)(C2)C3)o1] | AMINE: Int-7 ACID: 5- (methylsulfonyl- methyl)furan- 2-carboxylic acid | ¹H NMR (400 MHz, DMSO- d6): δ ppm: 9.11 (s, 1H), 8.25 (d, J = 2.0 Hz, 1H), 7.98 (d, J = 8.6 Hz, 1H), 7.55 (dd, J = 8.6, 2.0 Hz, 1H), 7.18 (d, J = 3.4 Hz, 1H), 6.67 (d, J = 3.4 Hz, 1H), 4.71 (s, 2H), 3.05 (s, 3H), 2.56 (s, 6H). MS obsd. (ESI+) [(M + H)+]: 437.0 |
| 14 | 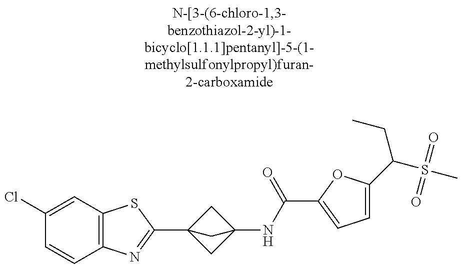 [SMILES: CCC(c1ccc(C(=O)NC23CC(c4nc5ccc(Cl)cc5s4)(C2)C3)o1)S(C)(=O)=O] | AMINE: Int-7 ACID: Int-13 | ¹H NMR (400 MHz, DMSO- d6): δ ppm: 9.11 (s, 1H), 8.26 (d, J = 2.0 Hz, 1H), 7.98 (d, J = 8.6 Hz, 1H), 7.55 (dd, J = 8.6, 2.0 Hz, 1H), 7.18 (d, J = 3.4 Hz, 1H), 6.73 (d, J = 3.4 Hz, 1H), 4.52-4.58 (m, 1H), 2.94 (s, 3H), 2.57 (s, 6H), 2.12-2.16 (m, 2H), 0.88 (t, J = 7.2 Hz, 3H). MS obsd. (ESI+) [(M + H)+]: 465.0 |
| 15 | 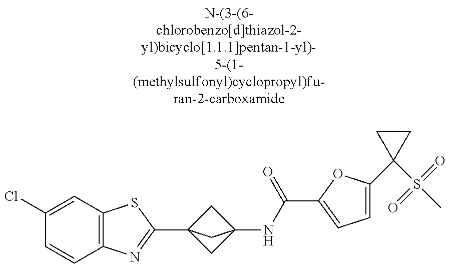 [SMILES: CS(=O)(=O)C1(c2ccc(C(=O)NC34CC(c5nc6ccc(Cl)cc6s5)(C3)C4)o2)CC1] | AMINE: Int-7 ACID: Int-9 | ¹H NMR (400 MHz, DMSO- d6): δ ppm: 9.13 (s, 1H), 8.25 (d, J = 2.0 Hz, 1H), 7.98 (d, J = 8.6 Hz, 1H), 7.55 (dd, J = 8.6, 2.0 Hz, 1H), 7.13 (d, J = 3.4 Hz, 1H), 6.80 (d, J = 3.4 Hz, 1H), 3.10 (s, 3H), 2.57 (s, 6H), 1.65- 1.75 (m, 2H), 1.53 (q, J = 5.2 Hz, 2H). MS obsd. (ESI+) [(M + H)+]: 463.0 |
| 16 | 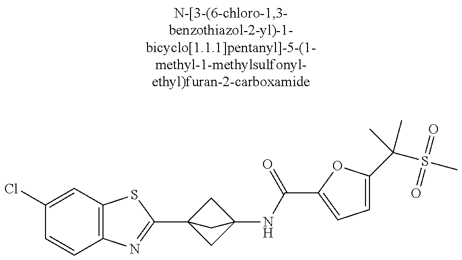 [SMILES: CC(C)(c1ccc(C(=O)NC23CC(c4nc5ccc(Cl)cc5s4)(C2)C3)o1)S(C)(=O)=O] | AMINE: Int-7 ACID: Int-14 | ¹H NMR (400 MHz, DMSO- d6): δ ppm: 9.10 (s, 1H), 8.25 (d, J = 2.0 Hz, 1H), 7.98 (d, J = 8.6 Hz, 1H), 7.55 (dd, J = 8.6, 2.0 Hz, 1H), 7.15 (d, J = 3.4 Hz, 1H), 6.78 (d, J = 3.4 Hz, 1H), 2.87 (s, 3H), 2.58 (s, 6H), 1.72 (s, 6H). MS obsd. (ESI+) [(M + H)+]: 465.0 |
| 17 | 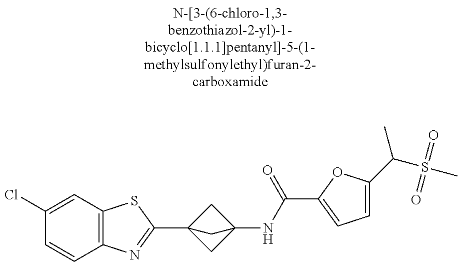 [SMILES: CC(c1ccc(C(=O)NC23CC(c4nc5ccc(Cl)cc5s4)(C2)C3)o1)S(C)(=O)=O] | AMINE: Int-7 ACID: Int-15 | ¹H NMR (400 MHz, DMSO- d6): δ ppm: 9.11 (s, 1H), 8.25 (d, J = 2.0 Hz, 1H), 7.98 (d, J = 8.6 Hz, 1H), 7.55 (dd, J = 8.6, 2.0 Hz, 1H), 7.16 (d, J = 3.6 Hz, 1H), 6.71 (d, J = 3.6 Hz, 1H), 4.74 (q, J = 7.2 Hz, 1H), 2.97 (s, 3H), 2.56 (s, 6H), 1.66 (d, J = 7.2 Hz, 3H). MS obsd. (ESI+) [(M + H)+]: 451.0 |
| 18 | 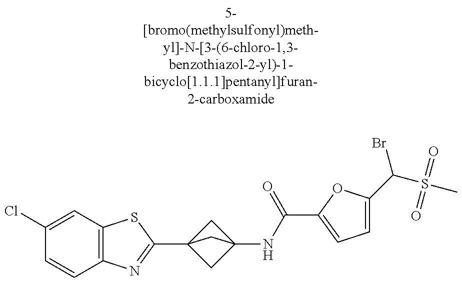 [SMILES: CS(=O)(=O)C(Br)c1ccc(C(=O)NC23CC(c4nc5ccc(Cl)cc5s4)(C2)C3)o1] | AMINE: Int-7 ACID: Int-16 | ¹H NMR (400 MHz, CDCl3) δ ppm: 7.93-7.98 (m, 1H), 7.83 (s, 1H), 7.43 (d, J = 6.8 Hz, 1H), 7.18 (s, 1H), 6.83-6.87 (m, 2H), 5.72-5.76 (m, 1H), 3.16 (s, 3H), 2.72 (s, 6H). MS obsd. (ESI+) [(M + H)+]: 516.9 |
| 19 | 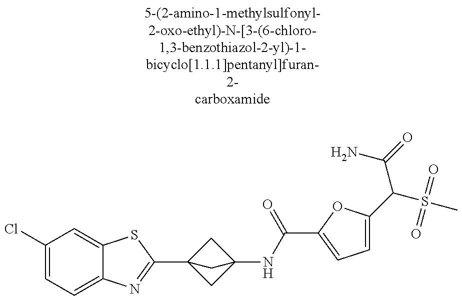 [SMILES: CS(=O)(=O)C(C(N)=O)c1ccc(C(=O)NC23CC(c4nc5ccc(Cl)cc5s4)(C2)C3)o1] | AMINE: Int-7 ACID: Int-17 | ¹H NMR (400 MHz, CD3OD) δ ppm: 8.02 (s, 1H), 7.90 (d, J = 8.8 Hz, 1H), 7.49-7.54 (m, 1H), 7.18 (d, J = 3.2 Hz, 1H), 6.89 (d, J = 3.2 Hz, 1H), 5.38 (s, 1H), 3.10 (s, 3H), 2.67 (s, 6H). MS obsd. (ESI+) [(M + H)+]: 480.0 |
| 20 | 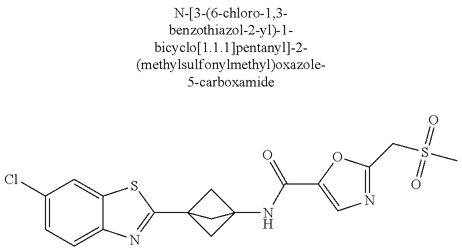 [SMILES: CS(=O)(=O)Cc1ncc(C(=O)NC23CC(c4nc5ccc(Cl)cc5s4)(C2)C3)o1] | AMINE: Int-7 ACID: Int-18 | ¹H NMR (400 MHz, CD3OD) δ ppm: 8.46 (s, 1H), 8.02 (d, J = 2.0 Hz, 1H), 7.90 (d, J = 8.8 Hz, 1H), 7.51 (dd, J = 8.8, 2.0 Hz, 1H), 4.80 (s, 2H), 3.14-3.18 (s, 3H), 2.67 (s, 6H). MS obsd. (ESI+) [(M + H)+]: 438.0 |
| 21 | 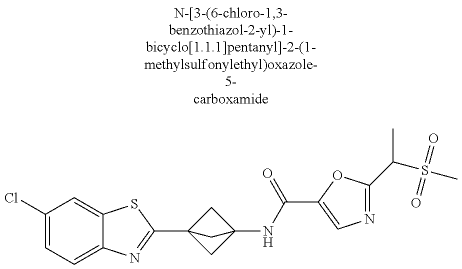 [SMILES: CC(c1ncc(C(=O)NC23CC(c4nc5ccc(Cl)cc5s4)(C2)C3)o1)S(C)(=O)=O] | AMINE: Int-7 ACID: Int-19 | ¹H NMR (400 MHz, CD3OD) δ (ppm) 7.93 (d, J = 2.0 Hz, 1H), 7.80 (d, J = 8.8 Hz, 1H), 7.67 (s, 1H), 7.42 (dd, J = 8.7, 2.1 Hz, 1H), 2.97-3.04 (m, 3H), 2.57 (s, 6H), 1.75 (d, J = 7.3 Hz, 3H) MS obsd. (ESI+) [(M + H)+]: 452.0 |
| 22 | 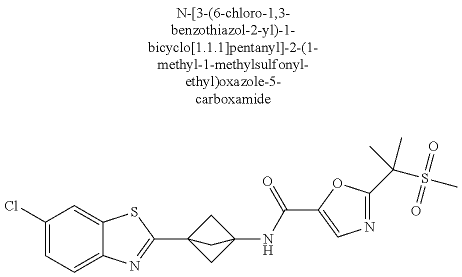 [SMILES: CC(C)(c1ncc(C(=O)NC23CC(c4nc5ccc(Cl)cc5s4)(C2)C3)o1)S(C)(=O)=O] | AMINE: Int-7 ACID: Int-20 | ¹H NMR (400 MHz, CD3OD) δ ppm: 8.46 (s, 1H), 8.02 (d, J = 2.0 Hz, 1H), 7.90 (d, J = 8.8 Hz, 1H), 7.51 (dd, J = 8.8, 2.0 Hz, 1H), 3.02 (s, 3H), 2.67 (s, 6H), 1.88 (s, 6H). MS obsd. (ESI+) [(M + H)+]: 466.1 |
| 23 | 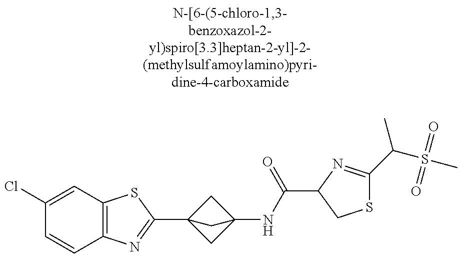 [SMILES: CC(C1=NC(C(=O)NC23CC(c4nc5ccc(Cl)cc5s4)(C2)C3)CS1)S(C)(=O)=O] | AMINE: Int-7 ACID: Int-21 | ¹H NMR (400 MHz, CD3OD): δ (ppm) 8.03 (d, J = 2.0 Hz, 1H), 7.91 (d, J = 8.8 Hz, 1H), 7.52 (dd, J = 8.8, 2.1 Hz, 1H), 5.17 (q, J = 8.5 Hz, 1H), 3.65-3.74 (m, 2H), 3.11 (s, 3H), 2.57-2.62 (m, 6H), 1.69-1.74 (m, 3H). MS obsd. (ESI+) [(M + H)+]: 470.0 |
| 24 | 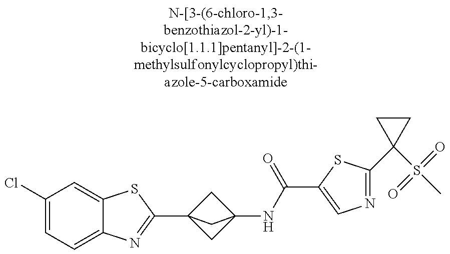 [SMILES: CS(=O)(=O)C1(c2ncc(C(=O)NC34CC(c5nc6ccc(Cl)cc6s5)(C3)C4)s2)CC1] | AMINE: Int-7 ACID: Int-22 | ¹H NMR (400 MHz, CD3OD) δ ppm: 8.26 (s, 1H), 8.02 (d, J = 2.0 Hz, 1H), 7.90 (d, J = 8.8 Hz, 1H), 7.51 (dd, J = 8.8, 2.0 Hz, 1H), 3.10 (s, 3H), 2.66 (s, 6H), 1.92-1.96 (m, 2H), 1.70-1.74 (m, 2H). MS obsd (ESI+) [(M + H)+]: 480.1 |
| 25 | 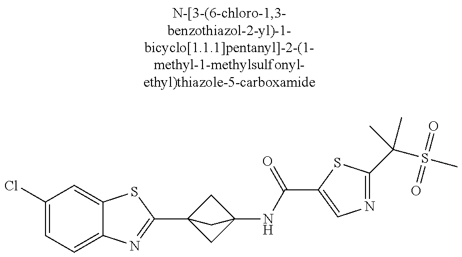 [SMILES: CC(C)(c1ncc(C(=O)NC23CC(c4nc5ccc(Cl)cc5s4)(C2)C3)s1)S(C)(=O)=O] | AMINE: Int-7 ACID: Int-23 | ¹H NMR (400 MHz, DMSO-d6) δ ppm: 9.53 (s, 1H), 8.43 (s, 1H), 8.26 (d, J = 2.0 Hz, 1H), 7.98 (d, J = 8.8 Hz, 1H), 7.55 (dd, J = 8.8, 2.2 Hz, 1H), 2.95 (s, 3H), 2.58 (s, 6H), 1.83 (s, 6H). MS obsd. (ESI+) [(M + H)+]: 482.0 |
| 26 | 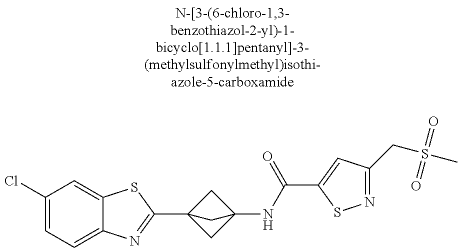 [SMILES: CS(=O)(=O)Cc1cc(C(=O)NC23CC(c4nc5ccc(Cl)cc5s4)(C2)C3)sn1] | AMINE: Int-7 ACID: Int-24 | ¹H NMR (400 MHz, CD3OD) δ ppm: 8.03 (d, J = 1.8 Hz, 1H), 7.88-7.92 (m, 2H), 7.84 (s, 1H), 4.70 (s, 2H), 2.99 (s, 3H), 2.67 (s, 6H) MS obsd. (ESI+) [(M + H)+]: 454.0 |
| 27 | 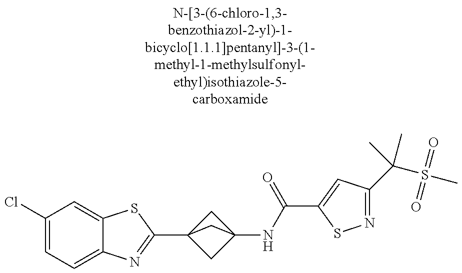 [SMILES: CC(C)(c1cc(C(=O)NC23CC(c4nc5ccc(Cl)cc5s4)(C2)C3)sn1)S(C)(=O)=O] | AMINE: Int-7 ACID: Int-25 | ¹H NMR (400 MHz, CD3OD) δ ppm: 8.02 (br. s, 2H), 7.90 (d, J = 8.8 Hz, 1H), 7.48-7.54 (m, 1H), 2.77 (s, 3H), 2.67 (s, 6H), 1.88 (s, 6H). MS obsd. (ESI+) [(M + H)+]: 482.0 |
| 28 | 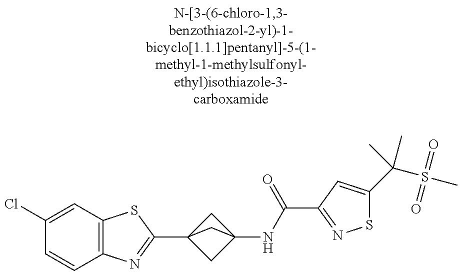 [SMILES: CC(C)(c1cc(C(=O)NC23CC(c4nc5ccc(Cl)cc5s4)(C2)C3)ns1)S(C)(=O)=O] | AMINE: Int-7 ACID: Int-26 | ¹H NMR (400 MHz, DMSO-d6) δ ppm: 9.51 (s, 1H), 8.25 (s, 1H), 7.98 (d, J = 7.8 Hz, 1H), 7.87 (s, 1H), 7.56 (d, J = 7.8 Hz, 1H), 2.94 (s, 3H), 2.57 (s, 6H), 1.83 (s, 6H). MS obsd. (ESI+) [(M + H)+]: 482.0 |
| 29 | 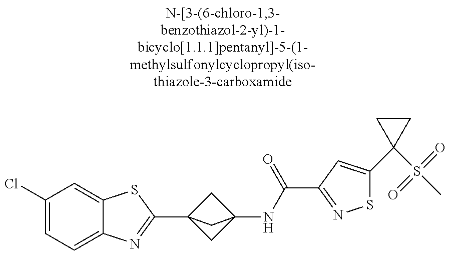 [SMILES: CS(=O)(=O)C1(c2cc(C(=O)NC34CC(c5nc6ccc(Cl)cc6s5)(C3)C4)ns2)CC1] | AMINE: Int-7 ACID: Int-27 | ¹H NMR (400 MHz, CDCl3) δ ppm: 7.92 (d, J = 8.8 Hz, 1H), 7.83 (d, J = 2.0 Hz, 1H), 7.76 (s, 1H), 7.71 (s, 1H), 7.43 (dd, J = 8.8, 2.0 Hz, 1H), 2.88 (s, 3H), 2.72 (s, 6H), 2.00-2.06 (m, 2H), 1.48-1.52 (m, 2H). MS obsd. (ESI+) [(M + H)+]: 480.0 |
| 30 | 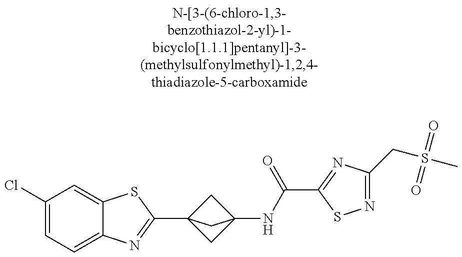 [SMILES: CS(=O)(=O)Cc1nsc(C(=O)NC23CC(c4nc5ccc(Cl)cc5s4)(C2)C3)n1] | AMINE: Int-7 ACID: Int-28 | ¹H NMR (400 MHz, DMSO-d6) δ ppm: 10.07 (s, 1H), 8.26 (d, J = 2.0 Hz, 1H), 7.99 (d, J = 8.8 Hz, 1H), 7.56 (dd, J = 8.8, 2.2 Hz, 1H), 5.00 (s, 2H), 3.26 (s, 3H), 2.62 (s, 6H). MS obsd. (ESI+) [(M + H)+]: 455.1 |
| 31 | 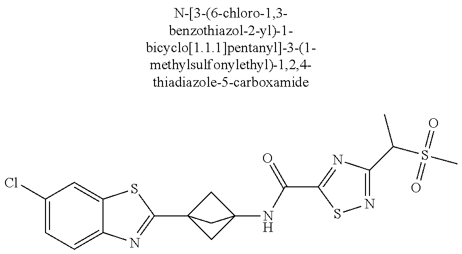 [SMILES: CC(c1nsc(C(=O)NC23CC(c4nc5ccc(Cl)cc5s4)(C2)C3)n1)S(C)(=O)=O] | AMINE: Int-7 ACID: Int-29 | ¹H NMR (400 MHz, DMSO-d6) δ ppm: 10.02 (s, 1H), 8.26 (d, J = 2.2 Hz, 1H), 7.99 (d, J = 8.8 Hz, 1H), 7.56 (dd, J = 8.8, 2.2 Hz, 1H), 5.03 (q, J = 7.2 Hz, 1H), 3.16 (s, 3H), 2.62 (s, 6H), 1.80 (d, J = 7.2 Hz, 3H). MS obsd. (ESI+) [(M + H)+]: 469.1 |
| 32 | 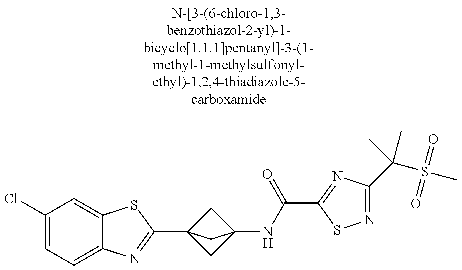 [SMILES: CC(C)(c1nsc(C(=O)NC23CC(c4nc5ccc(Cl)cc5s4)(C2)C3)n1)S(C)(=O)=O] | AMINE: Int-7 ACID: Int-30 | ¹H NMR (400 MHz, DMSO-d6) δ ppm: 9.94 (s, 1H), 8.26 (s, 1H), 7.99 (d, J = 8.6 Hz, 1H), 7.56 (d, J = 8.6 Hz, 1H), 3.10 (s, 3H), 2.64 (s, 6H), 1.88 (s, 6H). MS obsd. (ESI+) [(M + H)+]: 483.1 |
| 33 | 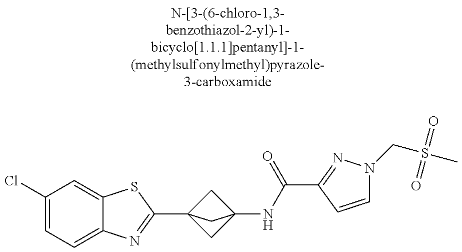 [SMILES: CS(=O)(=O)Cn1ccc(C(=O)NC23CC(c4nc5ccc(Cl)cc5s4)(C2)C3)n1] | AMINE: Int-7 ACID: Int-31 | ¹H NMR (400 MHz, DMSO-d6) δ ppm: 8.95 (s, 1H), 8.25 (d, J = 2.0 Hz, 1H), 7.89-8.05 (m, 2H), 7.55 (dd, J = 8.8, 2.2 Hz, 1H), 6.77 (d, J = 2.4 Hz, 1H), 5.79 (s, 2H), 3.14 (s, 3H), 2.53-2.62 (m, 6H). MS obsd. (ESI+) [(M + H)+]: 437.1 |
| 34 | 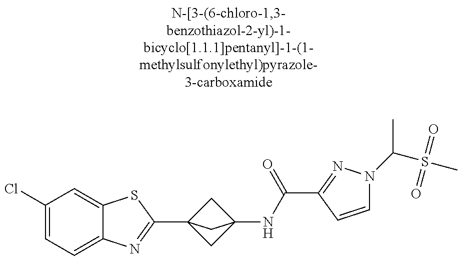 [SMILES: CC(n1ccc(C(=O)NC23CC(c4nc5ccc(Cl)cc5s4)(C2)C3)n1)S(C)(=O)=O] | AMINE: Int-7 ACID: Int-32 | ¹H NMR (400 MHz, DMSO-d6) δ ppm: 8.94 (s, 1H), 8.25 (d, J = 2.0 Hz, 1H), 7.98-8.02 (m, 2H), 7.55 (dd, J = 8.8, 2.2 Hz, 1H), 6.76 (d, J = 2.4 Hz, 1H), 5.90 (q, J = 7.2 Hz, 1H), 2.99 (s, 3H), 2.57 (s, 6H), 1.91 (d, J = 7.2 Hz, 3H). MS obsd. (ESI+) [(M + H)+]: 451.1 |
| 35 | 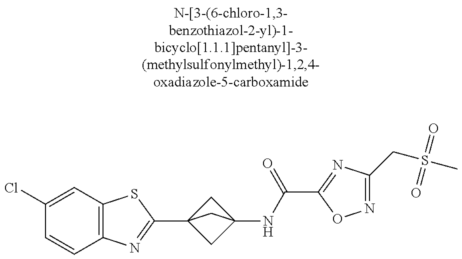 [SMILES: CS(=O)(=O)Cc1noc(C(=O)NC23CC(c4nc5ccc(Cl)cc5s4)(C2)C3)n1] | AMINE: Int-7 ACID: Int-33 | ¹H NMR (400 MHz, CD3OD) δ ppm: 8.03 (d, J = 2.0 Hz, 1H), 7.90 (d, J = 8.8 Hz, 1H), 7.52 (dd, J = 8.8, 2.0 Hz, 1H), 4.58 (s, 2H), 3.20 (s, 3H), 2.70 (s, 6H). MS obsd. (ESI+) [(M + H)+]: 439.0 |
| 36 | 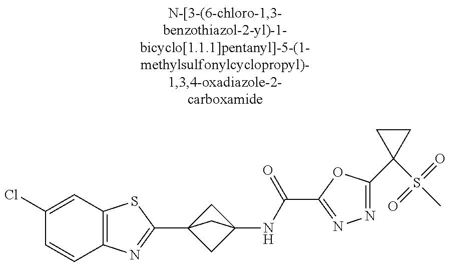 | AMINE: Int-7 ACID: Int-10 | ¹H NMR (400 MHz, CDCl3) δ ppm: 7.91 (d, J = 8.8 Hz, 1H), 7.83 (d, J = 2.0 Hz, 1H), 7.63 (s, 1H), 7.44 (dd, J = 8.8, 2.0 Hz, 1H), 3.29 (s, 3H), 2.73 (s, 6H), 2.07 (q, J = 5.4 Hz, 2H), 1.80 (q, J = 5.4 Hz, 2H). MS obsd. (ESI+) [(M + H)+]: 509.0 |
| 37 | 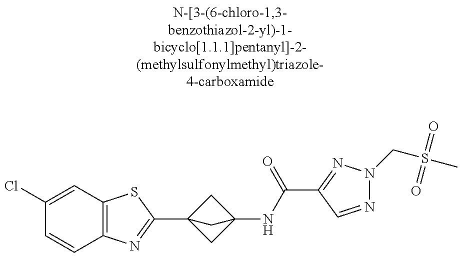 [SMILES: CS(=O)(=O)Cn1ncc(C(=O)NC23CC(c4nc5ccc(Cl)cc5s4)(C2)C3)n1] | AMINE: Int-7 ACID: Int-34 | ¹H NMR (400 MHz, DMSO-d6) δ ppm: 9.41 (s, 1H), 8.35 (s, 1H), 8.26 (d, J = 2.0 Hz, 1H), 7.98 (d, J = 8.6 Hz, 1H), 7.55 (dd, J = 8.6, 2.2 Hz, 1H), 6.17 (s, 2H), 3.21 (s, 3H), 2.59 (s, 6H). MS obsd. (ESI+) [(M + H)+]: 438.0 |
| 38 | 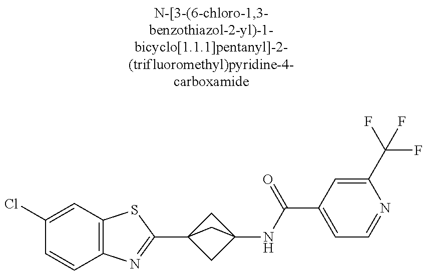 [SMILES: O=C(NC12CC(c3nc4ccc(Cl)cc4s3)(C1)C2)c1ccnc(C(F)(F)F)c1] | AMINE: Int-7 ACID: 2- (trifluoromethyl) isonicotinic acid | ¹H NMR (400 MHz, CD3OD) δ ppm: 8.87 (d, J = 5.2 Hz, 1H), 8.19 (s, 1H), 8.00-8.04 (m, 2H), 7.90 (d, J = 8.8 Hz, 1H), 7.52 (dd, J = 8.8, 1.8 Hz, 1H), 2.70 (s, 6H). MS obsd. (ESI+) [(M + H)+]: 424.0 |
| 39 | 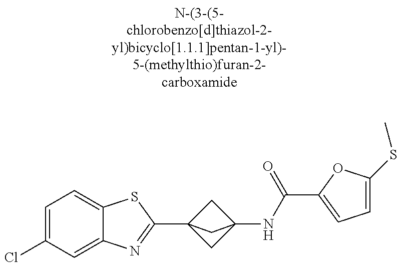 [SMILES: CSc1ccc(C(=O)NC23CC(c4nc5cc(Cl)ccc5s4)(C2)C3)o1] | AMINE: Int-8 ACID: Int-35 | ¹H NMR (DMSO-d6, 400 MHz): δ (ppm) 9.10 (s, 1H), 8.06-8.14 (m, 2H), 7.49 (dd, J = 8.7, 2.1 Hz, 1H), 7.13 (d, J = 3.4 Hz, 1H), 6.60 (d, J = 3.4 Hz, 1H), 2.56 (s, 6H), 2.52-2.53 (m, 3H) MS obsd. (ESI+) [(M + H)+]: 390.9 |
| 40 | 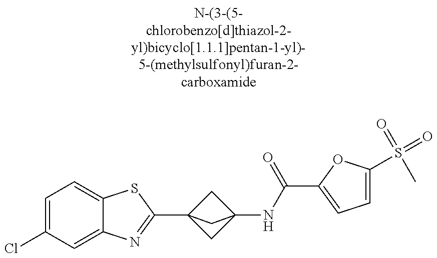 [SMILES: CS(=O)(=O)c1ccc(C(=O)NC23CC(c4nc5cc(Cl)ccc5s4)(C2)C3)o1] | AMINE: Int-8 ACID: Int-36 | ¹H NMR (DMSO-d6, 400 MHz): δ (ppm) 9.51 (s, 1H), 8.13 (d, J = 8.6 Hz, 1H), 8.08 (d, J = 2.0 Hz, 1H), 7.50 (dd, J = 8.6, 2.1 Hz, 1H), 7.39 (d, J = 3.7 Hz, 1H), 7.30 (d, J = 3.7 Hz, 1H), 3.40 (s, 3H), 2.59 (s, 6H) MS obsd. (ESI+) [(M + H)+]: 423.1 |
| 41 | 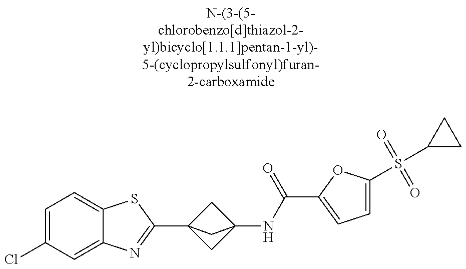 [SMILES: O=C(NC12CC(c3nc4cc(Cl)ccc4s3)(C1)C2)c1ccc(S(=O)(=O)C2CC2)o1] | AMINE: Int-1 ACID: Int-12 | ¹H NMR (DMSO-d6, 400 MHz): δ (ppm) 9.51 (s, 1H), 8.05-8.16 (m, 2H), 7.50 (d, J = 8.9 Hz, 1H), 7.39 (d, J = 3.7 Hz, 1H), 7.32 (d, J = 3.5 Hz, 1H), 2.92-3.03 (m, 1H), 2.59 (s, 6H), 1.12-1.28 (m, 4H) MS obsd. (ESI+) [(M + H)+]: 449.1 |
| 42 | 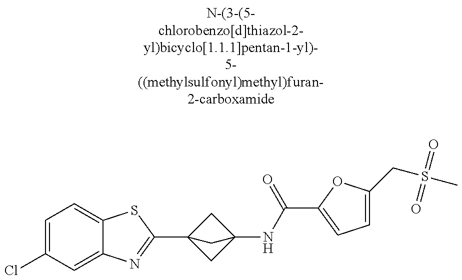 [SMILES: CS(=O)(=O)Cc1ccc(C(=O)NC23CC(c4nc5cc(Cl)ccc5s4)(C2)C3)o1] | AMINE: Int-8 ACID: 5- (methylsulfonyl- methyl)furan- 2-carboxylic acid | ¹H NMR (DMSO-d6, 400 MHz): δ (ppm) 9.11 (s, 1H), 8.13 (d, J = 8.7 Hz, 1H), 8.08 (d, J = 1.8 Hz, 1H), 7.49 (dd, J = 8.6, 2.1 Hz, 1H), 7.18 (d, J = 3.4 Hz, 1H), 6.67 (d, J = 3.4 Hz, 1H), 4.71 (s, 2H), 3.05 (s, 3H), 2.52-2.58 (m, 6H) MS obsd. (ESI+) [(M + H)+]: 437.1 |
| 43 | 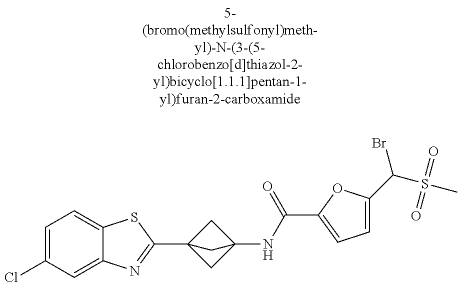 [SMILES: CS(=O)(=O)C(Br)c1ccc(C(=O)NC23CC(c4nc5cc(Cl)ccc5s4)(C2)C3)o1] | AMINE: Int-8 ACID: Int-16 | ¹H NMR (DMSO-d6, 400 MHz): δ (ppm) 9.23 (s, 1H), 8.13 (d, J = 8.8 Hz, 1H), 8.08 (d, J = 1.8 Hz, 1H), 7.50 (dd, J = 8.7, 2.1 Hz, 1H), 7.23 (d, J = 3.5 Hz, 1H), 6.93 (s, 1H), 6.91 (d, J = 3.5 Hz, 1H), 3.27 (s, 3H), 2.57 (s, 6H) MS obsd. (ESI+) [(M + H)+]: 514.9 |
| 44 | 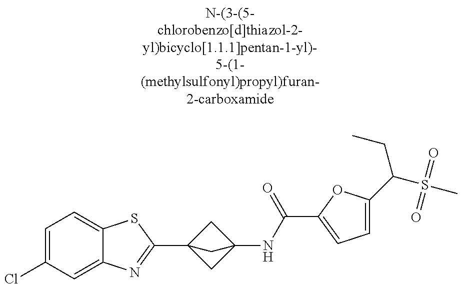 [SMILES: CCC(c1ccc(C(=O)NC23CC(c4nc5cc(Cl)ccc5s4)(C2)C3)o1)S(C)(=O)=O] | AMINE: Int-8 ACID: Int-13 | ¹H NMR (DMSO-d6, 400 MHz): δ (ppm) 9.11 (s, 1H), 8.13 (d, J = 8.7 Hz, 1H), 8.08 (d, J = 1.8 Hz, 1H), 7.50 (dd, J = 8.6, 2.1 Hz, 1H), 7.18 (d, J = 3.5 Hz, 1H), 6.73 (d, J = 3.4 Hz, 1H), 4.55 (dd, J = 11.2, 4.0 Hz, 1H), 2.94 (s, 3H), 2.52-2.59 (m, 6H), 2.06-2.26 (m, 2H), 0.88 (t, J = 7.3 Hz, 3H) MS obsd. (ESI+) [(M + H)+]: 465.2 |
| 45 | 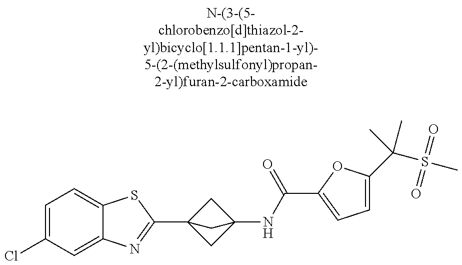 [SMILES: CC(C)(c1ccc(C(=O)NC23CC(c4nc5cc(Cl)ccc5s4)(C2)C3)o1)S(C)(=O)=O] | AMINE: Int-8 ACID: Int-14 | ¹H NMR (DMSO-d6, 400 MHz): δ (ppm) 9.10 (s, 1H), 8.13 (d, J = 8.7 Hz, 1H), 8.08 (d, J = 1.8 Hz, 1H), 7.50 (dd, J = 8.6, 2.1 Hz, 1H), 7.15 (d, J = 3.5 Hz, 1H), 6.78 (d, J = 3.5 Hz, 1H), 2.87 (s, 3H), 2.52-2.60 (m, 6H), 1.72 (s, 6H) MS obsd. (ESI+) [(M + H)+]: 465.1 |
| 46 | 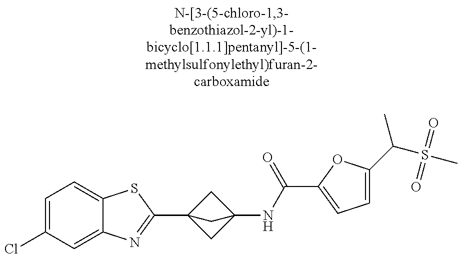 [SMILES: CC(c1ccc(C(=O)NC23CC(c4nc5cc(Cl)ccc5s4)(C2)C3)o1)S(C)(=O)=O] | AMINE: Int-8 ACID: Int-15 | ¹H NMR (DMSO-d6, 400 MHz): δ (ppm) 9.10 (s, 1H), 8.13 (d, J = 8.6 Hz, 1H), 8.08 (d, J = 1.8 Hz, 1H), 7.49 (dd, J = 8.6, 2.1 Hz, 1H), 7.16 (d, J = 3.4 Hz, 1H), 6.71 (d, J = 3.3 Hz, 1H), 4.73 (q, J = 7.2 Hz, 1H), 2.97 (s, 3H), 2.57 (s, 6H), 1.66 (d, J = 7.2 Hz, 3H) MS obsd. (ESI+) [(M + H)+]: 451.1 |
| 47 | 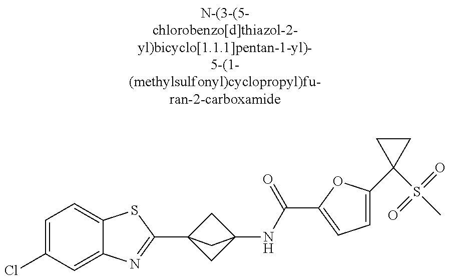 [SMILES: CS(=O)(=O)C1(c2ccc(C(=O)NC34CC(c5nc6cc(Cl)ccc6s5)(C3)C4)o2)CC1] | AMINE: Int-8 ACID: Int-9 | ¹H NMR (DMSO-d6, 400 MHz): δ (ppm) 9.12 (s, 1H), 8.13 (d, J = 8.6 Hz, 1H), 8.08 (d, J = 2.0 Hz, 1H), 7.49 (dd, J = 8.6, 2.1 Hz, 1H), 7.13 (d, J = 3.4 Hz, 1H), 6.79 (d, J = 3.5 Hz, 1H), 3.10 (s, 3H), 2.57 (s, 6H), 1.64-1.75 (m, 2H), 1.48-1.59 (m, 2H) MS obsd. (ESI+) [(M + H)+]: 463.1 |
| 48 | 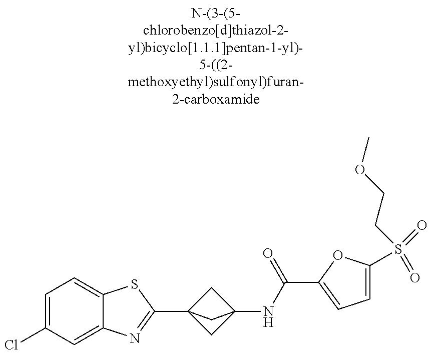 [SMILES: COCCS(=O)(=O)c1ccc(C(=O)NC23CC(c4nc5cc(Cl)ccc5s4)(C2)C3)o1] | AMINE: Int-8 ACID: Int-37 | ¹H NMR (DMSO-d6, 400 MHz): δ (ppm) 9.50 (s, 1H), 8.13 (d, J = 8.7 Hz, 1H), 8.08 (d, J = 2.0 Hz, 1H), 7.50 (dd, J = 8.6, 2.1 Hz, 1H), 7.38 (d, J = 3.7 Hz, 1H), 7.29 (d, J = 3.7 Hz, 1H), 3.65-3.79 (m, 4H), 3.13 (s, 3H), 2.59 (s, 6H) MS obsd (ESI+) [(M + H)+]: 467.1 |
| 49 | 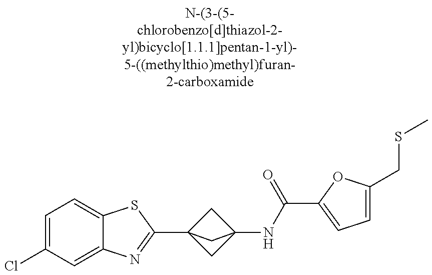 [SMILES: CSCc1ccc(C(=O)NC23CC(c4nc5cc(Cl)ccc5s4)(C2)C3)o1] | AMINE: Int-1 ACID: Int-38 | ¹H NMR (DMSO-d6, 400 MHz): δ (ppm) 9.03 (s, 1H), 8.12 (d, J = 8.6 Hz, 1H), 8.07 (d, J = 2.1 Hz, 1H), 7.49 (dd, J = 8.6, 2.1 Hz, 1H), 7.07 (d, J = 3.3 Hz, 1H), 6.43 (d, J = 3.3 Hz, 1H), 3.68-3.84 (m, 2H), 2.52- 2.60 (m, 6H), 2.06 (s, 3H) MS obsd. (ESI+) [(M + H)+]: 405.2 |
| 50 | 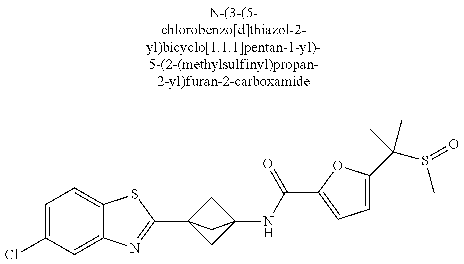 [SMILES: CS(=O)C(C)(C)c1ccc(C(=O)NC23CC(c4nc5cc(Cl)ccc5s4)(C2)C3)o1] | AMINE: Int-8 ACID: Int-39 | ¹H NMR (DMSO-d6, 400 MHz): δ (ppm) 9.06 (s, 1H), 8.13 (d, J = 8.7 Hz, 1H), 8.08 (d, J = 2.0 Hz, 1H), 7.50 (dd, J = 8.6, 2.1 Hz, 1H), 7.13 (d, J = 3.5 Hz, 1H), 6.63 (d, J = 3.5 Hz, 1H), 2.55-2.59 (m, 6H), 2.17 (s, 3H), 1.56 (d, J = 9.4 Hz, 6H) MS obsd. (ESI+) [(M + H)+]: 449.2 |
| 51 | 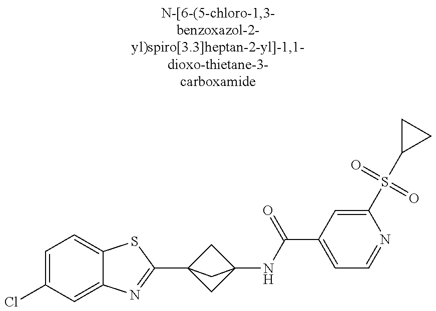 [SMILES: O=C(NC12CC(c3nc4cc(Cl)ccc4s3)(C1)C2)c1ccnc(S(=O)(=O)C2CC2)c1] | AMINE: Int-8 ACID: Int-40 | ¹H NMR (DMSO-d6, 400 MHz): δ (ppm) 9.84 (s, 1H), 8.99 (d, J = 4.9 Hz, 1H), 8.41 (s, 1H), 8.08-8.15 (m, 3H), 7.50 (dd, J = 8.7, 2.1 Hz, 1H), 2.95- 3.05 (m, 1H), 2.58-2.68 (m, 6H), 1.08-1.20 (m, 4H) MS obsd. (ESI+) [(M + H)+]: 460.0 |
| 52 | 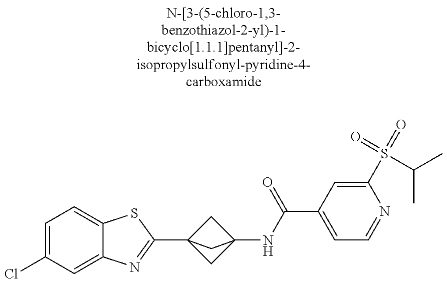 [SMILES: CC(C)S(=O)(=O)c1cc(C(=O)NC23CC(c4nc5cc(Cl)ccc5s4)(C2)C3)ccn1] | AMINE: Int-8 ACID: Int-41 | ¹H NMR (DMSO-d6, 400 MHz): δ (ppm) 9.87 (s, 1H), 8.99 (d, J = 4.9 Hz, 1H), 8.46 (d, J = 0.7 Hz, 1H), 8.08-8.16 (m, 3H), 7.50 (dd, J = 8.6, 2.1 Hz, 1H), 3.76 (quin, J = 6.8 Hz, 1H), 2.62 (s, 6H), 1.21 (d, J = 7.0 Hz, 6H) MS obsd. (ESI+) [(M + H)+]: 462.1 |
| 53 | 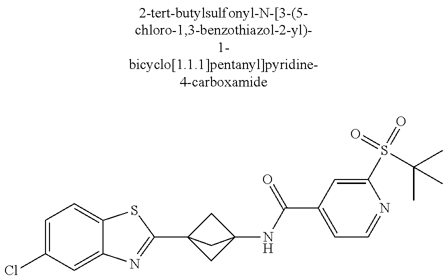 [SMILES: CC(C)(C)S(=O)(=O)c1cc(C(=O)NC23CC(c4nc5cc(Cl)ccc5s4)(C2)C3)ccn1] | AMINE: Int-8 ACID: Int-42 | ¹H NMR (DMSO-d6, 400 MHz): δ (ppm) 9.86 (s, 1H), 9.00 (d, J = 5.0 Hz, 1H), 8.46 (d, J = 0.9 Hz, 1H), 8.08-8.16 (m, 3H), 7.50 (dd, J = 8.6, 2.1 Hz, 1H), 2.62 (s, 6H), 1.33 (s, 6H) MS obsd. (ESI+) [(M + H)+]: 476.0 |
| 54 | 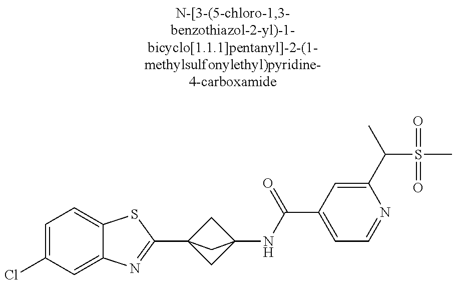 [SMILES: CC(c1cc(C(=O)NC23CC(c4nc5cc(Cl)ccc5s4)(C2)C3)ccn1)S(C)(=O)=O] | AMINE: Int-8 ACID: Int-43 | ¹H NMR (DMSO-d6, 400 MHz): δ (ppm) 9.59 (s, 1H), 8.76 (d, J = 5.0 Hz, 1H), 8.13 (d, J = 8.6 Hz, 1H), 8.09 (d, J = 2.0 Hz, 1H), 7.91 (s, 1H), 7.78 (dd, J = 5.1, 1.5 Hz, 1H), 7.50 (dd, J = 8.6, 2.1 Hz, 1H), 4.72 (q, J = 7.1 Hz, 1H), 2.93 (s, 3H), 2.61 (s, 6H), 1.73 (d, J = 7.1 Hz, 3H) MS obsd. (ESI+) [(M + H)+]: 462.0 |
| 55 | 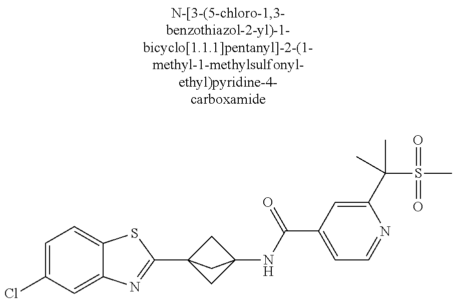 [SMILES: CC(C)(c1cc(C(=O)NC23CC(c4nc5cc(Cl)ccc5s4)(C2)C3)ccn1)S(C)(=O)=O] | AMINE: Int-8 ACID: Int-44 | ¹H NMR (DMSO-d6, 400 MHz): δ (ppm) 9.63 (s, 1H), 8.78 (d, J = 5.0 Hz, 1H), 8.05- 8.15 (m, 3H), 7.80 (dd, J = 5.0, 1.5 Hz, 1H), 7.50 (dd, J = 8.6, 2.1 Hz, 1H), 2.83 (s, 3H), 2.61- 2.68 (m, 6H), 1.83 (s, 6H) MS obsd. (ESI+) [(M + H)+]: 476.2 |

### Example 56-a and 56-b

The two enantiomers (Example 56-a and Example 56-b) were obtained through SFC [Instrument: MG II preparative SFC (SFC-1); Column: ChiralPak OD, 250×30 mm I.D., 5 μm; Mobile phase: A for CO2 and B for Ethanol; Gradient: B 50%; Flow rate: 50 mL/min; Back pressure: 100 bar Column temperature: 38° C.; Wavelength: 254 nm] chiral separation of N-[3-(6-chloro-1,3-benzothiazol-2-yl)-1-bicyclo[1.1.1]pentanyl]-5-(1-methylsulfonylethyl)furan-2-carboxamide (Example 17), Example 56-a was faster eluting than Example 56-b.

### N-[3-(6-chloro-1,3-benzothiazol-2-yl)-1-bicyclo[1.1.1]pentanyl]-5-[(1R)-1-methylsulfonylethyl]furan-2-carboxamide and N-[3-(6-chloro-1,3-benzothiazol-2-yl)-1-bicyclo[1.1.1]pentanyl]-5-[(1S)-1-methylsulfonylethyl]furan-2-carboxamide (Example 56-a and Example 56-b)

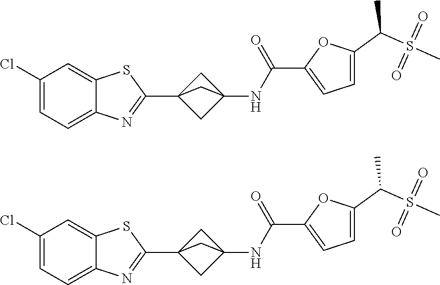 [SMILES: C[C@H](c1ccc(C(=O)NC23CC(c4nc5ccc(Cl)cc5s4)(C2)C3)o1)S(C)(=O)=O ; C[C@@H](c1ccc(C(=O)NC23CC(c4nc5ccc(Cl)cc5s4)(C2)C3)o1)S(C)(=O)=O]

Example 56-a: white solid. MS obsd. (ESI⁺) [(M+H)⁺]: 451.0. ¹H NMR (400 MHz, CD₃OD) δ ppm: 8.01 (d, J=1.8 Hz, 1H, 1H), 7.89 (d, J=8.8 Hz, 1H), 7.51 (d, J=8.8, 1.8 Hz, 1H), 7.15 (d, J=3.4 Hz, 1H), 6.71 (d, J=3.4 Hz, 1H), 4.65 (q, J=7.4 Hz, 1H), 2.92 (s, 3H), 2.66 (s, 6H), 1.77 (d, J=7.4 Hz, 3H).

Example 56-b: white solid. MS obsd. (ESI⁺) [(M+H)⁺]: 451.0. ¹H NMR (400 MHz, CD₃OD) δ ppm: 8.02 (d, J=1.8 Hz, 1H), 7.90 (d, J=8.8 Hz, 1H), 7.51 (dd, J=8.8, 2.0 Hz, 1H), 7.15 (d, J=3.6 Hz, 1H), 6.71 (d, J=3.6 Hz, 1H), 4.65 (q, J=7.4 Hz, 1H), 2.92 (s, 3H), 2.66 (s, 6H), 1.77 (d, J=7.4 Hz, 3H).

### Example 57-a and 57-b

The two enantiomers (Example 57-a and Example 57-b) were obtained through SFC [Instrument: SFC-80; Column: ChiralPak OD, 250×20 mm I.D., 5 μm; Mobile phase: A for CO2 and B for IPA; Gradient: B 40%; Flow rate: 50 mL/min; Back pressure: 100 bar Column temperature: 40° C.; Wavelength: 254 nm] chiral separation of N-[3-(6-chloro-1,3-benzothiazol-2-yl)-1-bicyclo[1.1.1]pentanyl]-5-(1-methylsulfonylethyl)furan-2-carboxamide (Example 46). Example 57-a was faster eluting than Example 57-b.

### N-[3-(5-chloro-1,3-benzothiazol-2-yl)-1-bicyclo[1.1.1]pentanyl]-5-[(1R)-1-methylsulfonylethyl]furan-2-carboxamide and N-[3-(5-chloro-1,3-benzothiazol-2-yl)-1-bicyclo[1.1.1]pentanyl]-5-[(1S)-1-methylsulfonylethyl]furan-2-carboxamide (Example 57-a and 57-b)

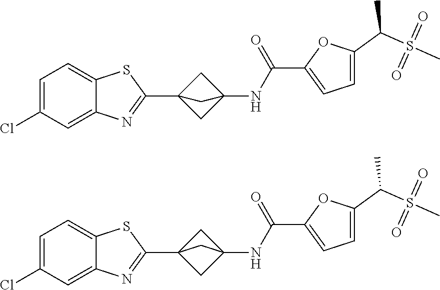 [SMILES: C[C@H](c1ccc(C(=O)NC23CC(c4nc5cc(Cl)ccc5s4)(C2)C3)o1)S(C)(=O)=O ; C[C@@H](c1ccc(C(=O)NC23CC(c4nc5cc(Cl)ccc5s4)(C2)C3)o1)S(C)(=O)=O]

Example 57-a: white solid. MS obsd. (ESI⁺) [(M+H)⁺]: 451.0. ¹H NMR (DMSO-d6, 400 MHz): δ (ppm) 9.10 (s, 1H), 8.13 (d, J=8.6 Hz, 1H), 8.08 (d, J=2.0 Hz, 1H), 7.49 (dd, J=8.6, 2.1 Hz, 1H), 7.16 (d, J=3.5 Hz, 1H), 6.71 (d, J=3.4 Hz, 1H), 4.73 (q, J=7.1 Hz, 1H), 2.97 (s, 3H), 2.57 (s, 6H), 1.66 (d, J=7.2 Hz, 3H).

Example 57-b: white solid. MS obsd. (ESI⁺) [(M+H)⁺]: 451.1. ¹H NMR (DMSO-d6, 400 MHz): δ (ppm) 9.11 (s, 1H), 8.13 (d, J=8.7 Hz, 1H), 8.08 (d, J=2.1 Hz, 1H), 7.49 (dd, J=8.7, 2.1 Hz, 1H), 7.16 (d, J=3.4 Hz, 1H), 6.71 (d, J=3.4 Hz, 1H), 4.73 (q, J=7.2 Hz, 1H), 2.97 (s, 3H), 2.57 (s, 6H), 1.66 (d, J=7.2 Hz, 3H).

### Example 58

### N-[3-(6-chloro-1,3-benzothiazol-2-yl)-1-bicyclo[1.1.1]pentanyl]-3-(1-methylsulfonylethyl)pyrazole-1-carboxamide

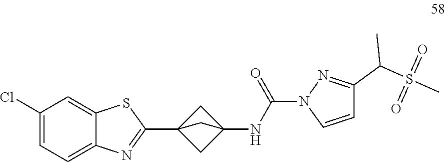 [SMILES: CC(c1ccn(C(=O)NC23CC(c4nc5ccc(Cl)cc5s4)(C2)C3)n1)S(C)(=O)=O]

The title compound was prepared according to the following scheme:

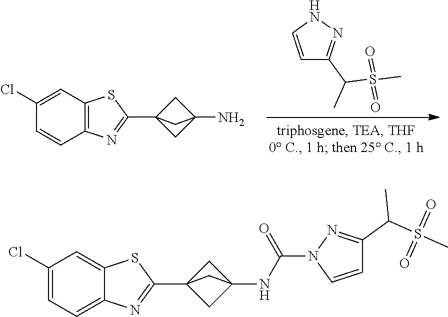 [SMILES: NC12CC(c3nc4ccc(Cl)cc4s3)(C1)C2 ; CC(c1cc[nH]n1)S(C)(=O)=O ; CC(c1ccn(C(=O)NC23CC(c4nc5ccc(Cl)cc5s4)(C2)C3)n1)S(C)(=O)=O]

To a solution of triphosgene (105 mg, 0.35 mmol) in THF (4 mL) was added 1-(6-chloro-1,3-benzothiazol-2-yl)bicyclo[1.1.1]pentan-3-amine (89.0 mg, 0.350 mmol) (Int-7, as the “AMINE-1” in Table 2) and TEA (0.3 mL, 2.13 mmol) at 0° C. After stirring at 0° C. for 1 h, 3-(1-methylsulfonylethyl)-1H-pyrazole (Int-45, as the “AMINE-2” in Table 2) (62 mg, 0.35 mmol) was added. The reaction mixture was warmed to 25° C. and stirred for another 1 h. The resulting solution was concentrated in vacuum. The residue was purified by prep-HPLC (Chromatographic columns: Kromasil-C18 100×21.2 mm 5 um Mobile Phase: ACN-H₂O (0.1% FA) Gradient: 50%-60%) to give N-[1-(6-chloro-1,3-benzothiazol-2-yl)-3-bicyclo[1.1.1]pentanyl]-3-(1-methylsulfonylethyl)pyrazole-1-carboxamide (5.4 mg) as a white solid.

MS obsd. (ESI⁺) [(M+H)⁺]: 451.1. ¹H NMR (400 MHz, CD₃OD) δ ppm: 8.26 (d, J=2.8 Hz, 1H), 8.03 (d, J=2.0 Hz, 1H), 7.90 (d, J=8.8 Hz, 1H), 7.51 (dd, J=8.8, 2.0 Hz, 1H), 6.62 (d, J=2.8 Hz, 1H), 4.73-4.81 (m, 1H), 2.88 (s, 3H), 2.68 (s, 6H), 1.77 (d, J=7.2 Hz, 3H).

The following Example 59 to Example 63 were prepared in analogy to the procedure described for the preparation of Example 58, replacing 1-(6-chloro-1,3-benzothiazol-2-yl)bicyclo[1.1.1]pentan-3-amine (Int-7) with “AMINE-1”, and replacing 3-(1-methylsulfonylethyl)-1H-pyrazole (Int-45) with “AMINE-2”. The “AMINE-1” and “AMINE-2” are the reagents indicated in Table 2

TABLE 2
Compounds synthesis and characterization

| Example | Compounds Name and | AMINE-1 and | |
|---|---|---|---|
| No. | Structure | AMINE-2 | ¹H NMR and (ESI⁺) |
| 59 | 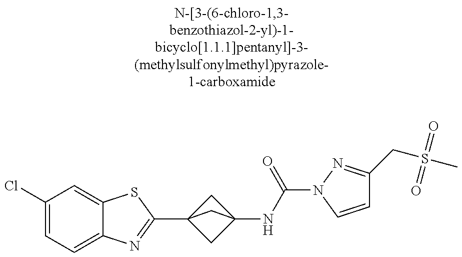 [SMILES: CS(=O)(=O)Cc1ccn(C(=O)NC23CC(c4nc5ccc(Cl)cc5s4)(C2)C3)n1] | AMINE-1: Int-7 AMINE-2: Int-46 | ¹H NMR (400 MHz, DMSO-d6) δ ppm: 9.35 (s, 1H), 8.32 (d, J = 2.8 Hz, 1H), 8.26 (d, J = 2.2 Hz, 1H), 7.98 (d, J = 8.8 Hz, 1H), 7.56 (dd, J = 8.8, 2.2 Hz, 1H), 6.60 (d, J = 2.8 Hz, 1H), 4.56 (s, 2H), 3.06 (s, 3H), 2.58 (s, 6H). MS obsd. (ESI⁺) [(M + H)⁺]: 437.1 |
| 60 | 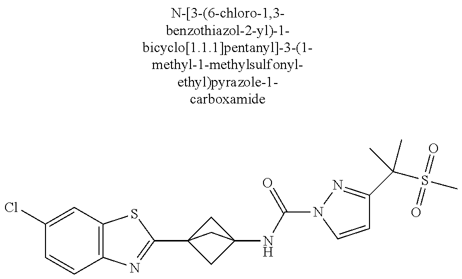 [SMILES: CC(C)(c1ccn(C(=O)NC23CC(c4nc5ccc(Cl)cc5s4)(C2)C3)n1)S(C)(=O)=O] | AMINE-1: Int-7 AMINE-2: Int-47 | ¹H NMR (400 MHz, CDCl3) δ ppm: 8.23 (d, J = 2.8 Hz, 1H), 7.92 (d, J = 8.8 Hz, 1H), 7.84 (d, J = 2.0 Hz, 1H), 7.56 (s, 1H), 7.44 (dd, J = 8.8, 2.0 Hz, 1H), 6.65 (d, J = 2.8 Hz, 1H), 2.73 (s, 6H), 2.71 (s, 3H), 1.84 (s, 6H). MS obsd. (ESI⁺) [(M + H)⁺]: 465.1 |
| 61 | 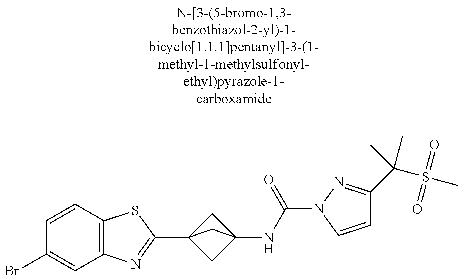 [SMILES: CC(C)(c1ccn(C(=O)NC23CC(c4nc5cc(Br)ccc5s4)(C2)C3)n1)S(C)(=O)=O] | AMINE-1: Int-2 AMINE-2: Int-47 | ¹H NMR (400 MHz, CDCl3) δ ppm: 8.22 (d, J = 2.4 Hz, 1H), 8.16 (s, 1H), 7.71 (d, J = 8.6 Hz, 1H), 7.49-7.53 (m, 2H), 6.65 (d, J = 2.4 Hz, 1H), 2.73 (s, 6H), 2.70 (s, 3H), 1.83 (s, 6H). MS obsd. (ESI+) [(M + H)+]: 509.1 |
| 62 | 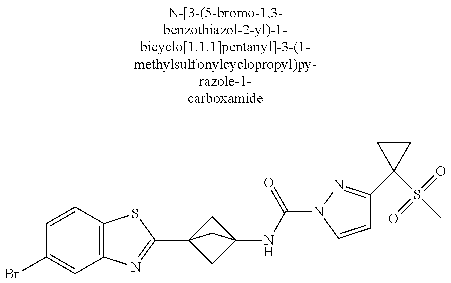 [SMILES: CS(=O)(=O)C1(c2ccn(C(=O)NC34CC(c5nc6cc(Br)ccc6s5)(C3)C4)n2)CC1] | AMINE-1: Int-2 AMINE-2: Int-48 | ¹H NMR (400 MHz, CDCl3) δ ppm: 8.22 (d, J = 2.8 Hz, 1H), 8.18 (d, J = 2.0 Hz, 1H), 7.73 (d, J = 8.6 Hz, 1H), 7.48-7.55 (m, 2H), 6.73 (d, J = 2.8 Hz, 1H), 2.92 (s, 3H), 2.73 (s, 6H), 1.84- 1.88 (m, 2H), 1.47-1.51 (m, 2H). MS obsd. (ESI+) [(M + H)+]: 507.1 |
| 63 | 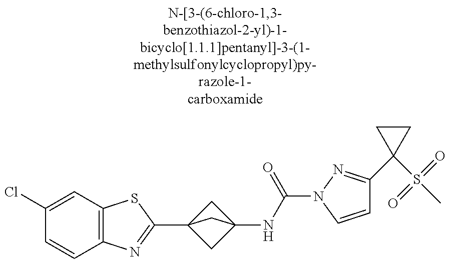 [SMILES: CS(=O)(=O)C1(c2ccn(C(=O)NC34CC(c5nc6ccc(Cl)cc6s5)(C3)C4)n2)CC1] | AMINE-1: Int-7 AMINE-2: Int-48 | ¹H NMR (400 MHz, CDCl3) δ ppm: 8.21 (d, J = 2.8 Hz, 1H), 7.92 (d, J = 8.8 Hz, 1H), 7.84 (d, J = 2.0 Hz, 1H), 7.52 (s, 1H), 7.44 (dd, J = 8.8, 2.0 Hz, 1H), 6.72 (d, J = 2.8 Hz, 1H), 2.91 (s, 3H), 2.72 (s, 6H), 1.77-1.82 (m, 2H), 1.46-1.50 (m, 2H). MS obsd. (ESI+)[(M + H)+]: 463.1 |

Example 64

### N-(3-(5-chlorobenzo[d]thiazol-2-yl)bicyclo[1.1.1]pentan-1-yl)-5-(S-methylsulfonimidoyl)furan-2-carboxamide

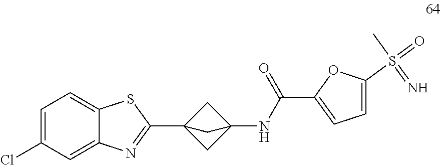 [SMILES: CS(=N)(=O)c1ccc(C(=O)NC23CC(c4nc5cc(Cl)ccc5s4)(C2)C3)o1]

The title compound was prepared according to the following scheme:

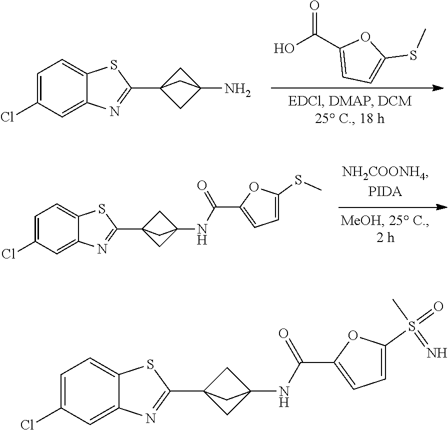

### Step 1: Preparation of N-(3-(5-chlorobenzo[d]thiazol-2-yl)bicyclo[1.1.1]pentan-1-yl)-5-(methylthio)furan-2-carboxamide

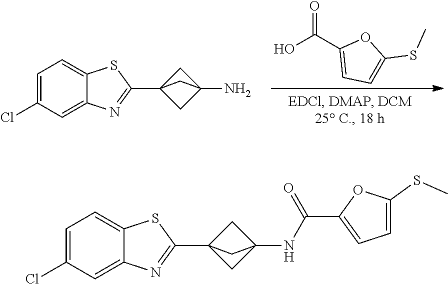

To a mixture of 3-(5-chlorobenzo[d]thiazol-2-yl)bicyclo[1.1.1]pentan-1-amine (100 mg, 399 μmol, Int-8) and 5-(methylthio)furan-2-carboxylic acid (69 mg, 439 μmol, Int-35) in DCM (4 ml) was added EDCI (115 mg, 598 μmol) and DMAP (73 mg, 598 μmol). Then the reaction was stirred at 25° C. for 18 h. The reaction solution was purified by silica gel column eluted with DCM/MeOH=60/1 to give N-(3-(5-chlorobenzo[d]thiazol-2-yl)bicyclo[1.1.1]pentan-1-yl)-5-(methylthio)furan-2-carboxamide (130 mg) as a white solid.

MS obsd. (ESI⁺) [(M+H)⁺]: 390.9.

### Step 2: Preparation of [1-(2-trimethylsilylethoxymethyl)pyrazol-3-yl]methanol

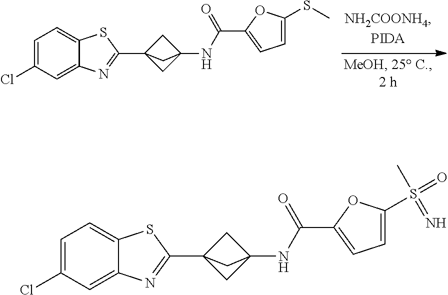 [SMILES: CSc1ccc(C(=O)NC23CC(c4nc5cc(Cl)ccc5s4)(C2)C3)o1 ; CS(=N)(=O)c1ccc(C(=O)NC23CC(c4nc5cc(Cl)ccc5s4)(C2)C3)o1]

A mixture of N-(3-(5-chlorobenzo[d]thiazol-2-yl)bicyclo[1.1.1]pentan-1-yl)-5-(methylthio)furan-2-carboxamide (100 mg, 256 μmol), PIDA (173 mg, 537 μmol) and NH₂COONH₄ (37 mg, 384 μmol) in MeOH (5 ml) was stirred at 25° C. for 2 h. The reaction was concentrated in vacuum and the residue was purified by silica gel column eluted with DCM/MeOH=40/1 to give N-(3-(5-chlorobenzo[d]thiazol-2-yl)bicyclo[1.1.1]pentan-1-yl)-5-(S-methylsulfonimidoyl)furan-2-carboxamide (95 mg) (Example 64) as a white solid.

MS obsd. (ESI⁺) [(M+H)⁺]: 422.1. ¹H NMR (DMSO-d6, 400 MHz): δ (ppm) 9.43 (s, 1H), 8.13 (d, J=8.7 Hz, 1H), 8.08 (d, J=2.0 Hz, 1H), 7.50 (dd, J=8.6, 2.1 Hz, 1H), 7.23 (d, J=3.7 Hz, 1H), 7.16 (d, J=3.5 Hz, 1H), 4.88 (s, 1H), 3.14-3.23 (m, 3H), 2.59 (s, 6H).

### Example 65

### N-[3-(6-chloro-1,3-benzothiazol-2-yl)-1-bicyclo[1.1.1]pentanyl]-5-(cyclopropylsulfonimidoyl)furan-2-carboxamide

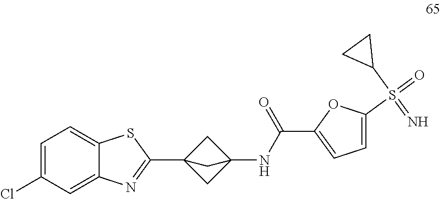 [SMILES: N=S(=O)(c1ccc(C(=O)NC23CC(c4nc5cc(Cl)ccc5s4)(C2)C3)o1)C1CC1]

The title compound was prepared in analogy to the procedure described for the preparation of Example 64, by using 3-(6-chloro-1,3-benzothiazol-2-yl)bicyclo[1.1.1]pentan-1-amine (Int-7) instead of 3-(5-chlorobenzo[d]thiazol-2-yl)bicyclo[1.1.1]pentan-1-amine (Int-8) and 5-cyclopropylsulfanylfuran-2-carboxylic acid (Int-49) instead of 5-(methylthio)furan-2-carboxylic acid (Int-35). The product was purified by preparative HPLC to afford Example 65 as a white solid.

MS obsd. (ESI⁺) [(M+H)⁺]: 448.1. ¹H NMR (400 MHz, CD3OD) δ ppm: 8.03 (s, 1H), 7.90 (d, J=8.6 Hz, 1H), 7.52 (d, J=8.6 Hz, 1H), 7.22-7.26 (m, 2H), 3.72-3.76 (m, 1H), 2.67 (s, 6H), 0.91-1.55 (m, 4H).

### Example 66

### N-[3-(6-chloro-1,3-benzothiazol-2-yl)-1-bicyclo[1.1.1]pentanyl]-5-(cyclopropylmethylsulfonimidoyl)furan-2-carboxamide

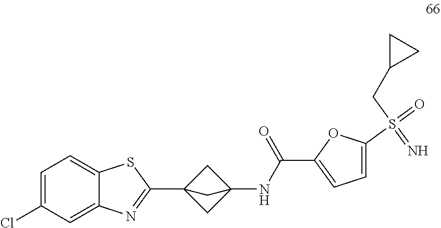 [SMILES: N=S(=O)(CC1CC1)c1ccc(C(=O)NC23CC(c4nc5cc(Cl)ccc5s4)(C2)C3)o1]

The title compound was prepared in analogy to the procedure described for the preparation of Example 64, by using 3-(6-chloro-1,3-benzothiazol-2-yl)bicyclo[1.1.1]pentan-1-amine (Int-7) instead of 3-(5-chlorobenzo[d]thiazol-2-yl)bicyclo[1.1.1]pentan-1-amine (Int-8) and 5-(cyclopropylmethylsulfanyl)furan-2-carboxylic acid (Int-45) instead of 5-(methylthio)furan-2-carboxylic acid (Int-35). The product was purified by preparative HPLC to afford Example 66 as a white solid.

MS obsd. (ESI⁺) [(M+H)⁺]: 462.1. ¹H NMR (400 MHz, DMSO-d6) δ ppm: 9.42 (s, 1H), 8.27 (d, J=2.0 Hz, 1H), 8.00 (d, J=8.8 Hz, 1H), 7.57 (dd, J=8.8, 2.0 Hz, 1H), 7.27 (d, J=3.6 Hz, 1H), 7.20 (d, J=3.6 Hz, 1H), 4.89 (s, 1H), 3.26 (d, J=7.2 Hz, 2H), 2.52 (s, 4H), 2.09 (s, 2H), 0.96-1.00 (m, 1H), 0.45 (q, J=5.6 Hz, 2H), 0.11 (q, J=5.6 Hz, 2H).

### Example 67

### N-[3-(6-chloro-1,3-benzothiazol-2-yl)-1-bicyclo[1.1.1]pentanyl]-2-(1-methylsulfonylcyclopropyl)oxazole-5-carboxamide

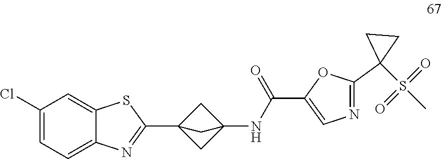 [SMILES: CS(=O)(=O)C1(c2ncc(C(=O)NC34CC(c5nc6ccc(Cl)cc6s5)(C3)C4)o2)CC1]

The title compound was prepared according to the following scheme:

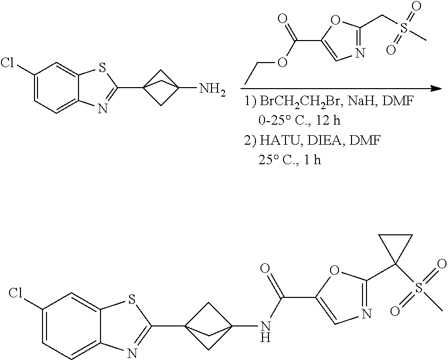 [SMILES: NC12CC(c3nc4ccc(Cl)cc4s3)(C1)C2 ; CCOC(=O)c1cnc(CS(C)(=O)=O)o1 ; CS(=O)(=O)C1(c2ncc(C(=O)NC34CC(c5nc6ccc(Cl)cc6s5)(C3)C4)o2)CC1]

To a solution of ethyl 2-(methylsulfonylmethyl)oxazole-5-carboxylate (50 mg, 0.21 mmol) in DMF (3 mL) was added sodium hydride (13 mg, 0.54 mmol, 60% in oil) at 0° C. After stirred at 0° C. for 0.5 h, 1,2-dibromoethane (60 mg, 0.32 mmol) was added. The reaction mixture was stirred at 25° C. for 12 h. To the stirred solution, HATU (36.18 mg, 0.100 mmol), DIEA (33.54 mg, 0.260 mmol) and 1-(6-chloro-1,3-benzothiazol-2-yl)bicyclo[1.1.1]pentan-3-amine (22 mg, 0.090 mmol) were added. The reaction mixture was stirred at 25° C. for 1 h. The resulted mixture was purified by prep-HPLC to give N-[1-(6-chloro-1,3-benzothiazol-2-yl)-3-bicyclo[1.1.1]pentanyl]-2-(1-methylsulfonylcyclopropyl)oxazole-5-carboxamide (2.8 mg) (Example 67) as a white solid.

MS obsd. (ESI⁺) [(M+H)⁺]: 464.0. ¹H NMR (400 MHz, CD3OD) δ ppm: 8.41 (s, 1H), 8.02 (d, J=2.0 Hz, 1H), 7.90 (d, J=8.8 Hz, 1H), 7.51 (dd, J=8.8, 2.0 Hz, 1H), 3.29 (s, 3H), 2.67 (s, 6H), 1.87-1.90 (m, 2H), 1.73-1.77 (m, 2H).

### Example 68

### N-[3-(6-chloro-1,3-benzothiazol-2-yl)-1-bicyclo[1.1.1]pentanyl]-5-(1-methylsulfonylcyclopropyl)thiazole-2-carboxamide

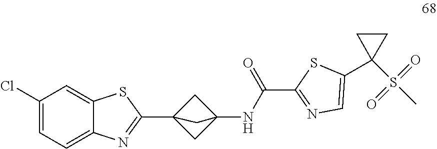 [SMILES: CS(=O)(=O)C1(c2cnc(C(=O)NC34CC(c5nc6ccc(Cl)cc6s5)(C3)C4)s2)CC1]

The title compound was prepared in analogy to the procedure described for the preparation of Example 67, by using methyl 5-(methylsulfonylmethyl)thiazole-2-carboxylate (Int-51) instead of ethyl 2-(methylsulfonylmethyl)oxazole-5-carboxylate. The product was purified by preparative HPLC to afford Example 68 as a white solid.

MS obsd. (ESI⁺) [(M+H)⁺]: 480.0. ¹H NMR (400 MHz, CD3OD) δ ppm: 8.05 (s, 1H), 8.03 (s, 1H), 7.90 (d, J=8.8 Hz, 1H), 7.51 (d, J=8.8 Hz, 1H), 2.93 (s, 3H), 2.68 (s, 6H), 1.87-1.91 (m, 2H), 1.52-1.56 (m, 2H).

### Example 69

### N-[3-(6-chloro-1,3-benzothiazol-2-yl)-1-bicyclo[1.1.1]pentanyl]-5-(1-methylsulfonylcyclopropyl)oxazole-2-carboxamide

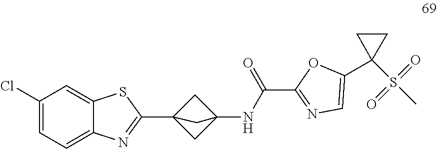 [SMILES: CS(=O)(=O)C1(c2cnc(C(=O)NC34CC(c5nc6ccc(Cl)cc6s5)(C3)C4)o2)CC1]

The title compound was prepared in analogy to the procedure described for the preparation of Example 67, by using ethyl 5-(methylsulfonylmethyl)oxazole-2-carboxylate (Int-52) instead of ethyl 2-(methylsulfonylmethyl)oxazole-5-carboxylate. The product was purified by preparative HPLC to afford Example 69 as a white solid.

MS obsd. (ESI⁺) [(M+H)⁺]: 464.0. ¹H NMR (400 MHz, CD₃OD) δ ppm: 8.02 (d, J=2.0 Hz, 1H), 7.90 (d, J=8.8 Hz, 1H), 7.51 (dd, J=8.8, 2.0 Hz, 1H), 7.47 (s, 1H), 3.08 (s, 3H), 2.68 (s, 6H), 1.81-1.90 (m, 2H), 1.53-1.63 (m, 2H).

### Example 70

### N-[3-(6-chloro-1,3-benzothiazol-2-yl)-1-bicyclo[1.1.1]pentanyl]-5-(cyclopropylmethylsulfonyl)furan-2-carboxamide

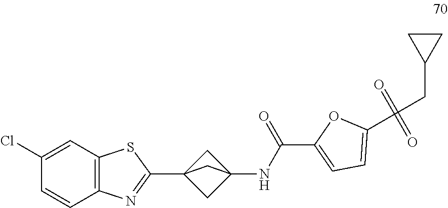

The title compound was prepared according to the following scheme:

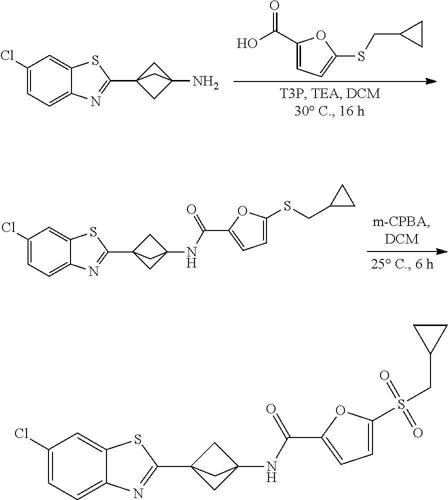 [SMILES: NC12CC(c3nc4ccc(Cl)cc4s3)(C1)C2 ; O=C(O)c1ccc(SCC2CC2)o1 ; O=C(NC12CC(c3nc4ccc(Cl)cc4s3)(C1)C2)c1ccc(SCC2CC2)o1 ; O=C(NC12CC(c3nc4ccc(Cl)cc4s3)(C1)C2)c1ccc(S(=O)(=O)CC2CC2)o1]

### Step 1: Preparation of N-[3-(6-chloro-1,3-benzothiazol-2-yl)-1-bicyclo[1.1.1]pentanyl]-5-(cyclopropylmethylsulfanyl)furan-2-carboxamide

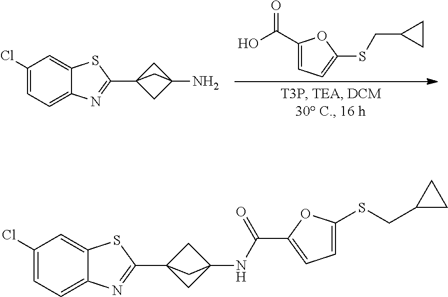 [SMILES: NC12CC(c3nc4ccc(Cl)cc4s3)(C1)C2 ; O=C(O)c1ccc(SCC2CC2)o1 ; O=C(NC12CC(c3nc4ccc(Cl)cc4s3)(C1)C2)c1ccc(SCC2CC2)o1]

To a solution of 1-(6-chloro-1,3-benzothiazol-2-yl)bicyclo[1.1.1]pentan-3-amine (414 mg, 1.65 mmol) and 5-(cyclopropylmethylsulfanyl)furan-2-carboxylic acid (654 mg, 3.3 mmol, Int-50) in DCM (30 mL) was added triethylamine (1.38 mL, 9.9 mmol) and T₃P (6.3 g, 9.9 mmol). The reaction mixture was stirred at 30° C. for 16 h. The resulting solution was concentrated in vacuum. The residue was purified by silica gel column eluted with PE/EtOAc=4/1 to give N-[3-(6-chloro-1,3-benzothiazol-2-yl)-1-bicyclo[1.1.1]pentanyl]-5-(cyclopropylmethylsulfanyl) furan-2-carboxamide (610 mg) as an off-white solid.

MS obsd. (ESI⁺) [(M+H)⁺]: 433.0.

### Step 2: Preparation of N-[3-(6-chloro-1,3-benzothiazol-2-yl)-1-bicyclo[1.1.1]pentanyl]-5-(cyclopropylmethylsulfonyl)furan-2-carboxamide

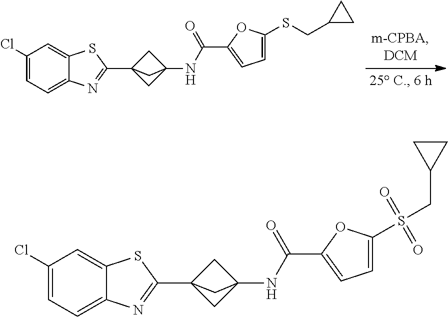 [SMILES: O=C(NC12CC(c3nc4ccc(Cl)cc4s3)(C1)C2)c1ccc(SCC2CC2)o1 ; O=C(NC12CC(c3nc4ccc(Cl)cc4s3)(C1)C2)c1ccc(S(=O)(=O)CC2CC2)o1]

To a solution of N-[1-(6-chloro-1,3-benzothiazol-2-yl)-3-bicyclo[1.1.1]pentanyl]-5-(cyclopropylmethylsulfanyl)furan-2-carboxamide (150 mg, 0.35 mmol) in DCM (15 mL) was added m-CPBA (132 mg, 0.77 mmol). The reaction was stirred at 25° C. for 6 h. After concentration, the residue was purified by prep-HPLC (Chromatographic columns: Kromasil-C18 100×21.2 mm 5 um Mobile Phase: ACN-H₂O (0.1% FA) Gradient: 55-65) to give N-[3-(6-chloro-1,3-benzothiazol-2-yl)-1-bicyclo[1.1.1]pentanyl]-5-(cyclopropylmethylsulfonyl)furan-2-carboxamide (129 mg) (Example 70) as white solid.

MS obsd. (ESI⁺) [(M+H)⁺]: 463.0. ¹H NMR (400 MHz, DMSO-d6) δ ppm: 9.49 (s, 1H), 8.26 (d, J=2.0 Hz, 1H), 7.98 (d, J=8.8 Hz, 1H), 7.56 (dd, J=8.8, 2.0 Hz, 1H), 7.42 (d, J=3.6 Hz, 1H), 7.33 (d, J=3.6 Hz, 1H), 3.44 (d, J=7.2 Hz, 2H), 2.59 (s, 6H), 0.92-0.96 (m, 1H), 0.59-0.41 (m, 2H), 0.16 (q, J=4.6 Hz, 2H).

### Example 71

### N-[3-(6-chloro-1,3-benzothiazol-2-yl)-1-bicyclo[1.1.1]pentanyl]-5-(cyclopropylmethylsulfinyl)furan-2-carboxamide

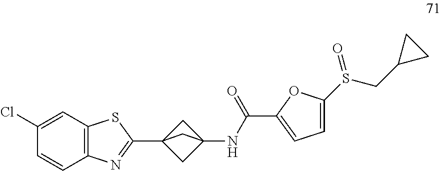 [SMILES: O=C(NC12CC(c3nc4ccc(Cl)cc4s3)(C1)C2)c1ccc(S(=O)CC2CC2)o1]

The title compound was prepared in analogy to the procedure described for the preparation of Example 70, by decreasing the equivalent of the m-CPBA from 2 to 1.1. The product was purified by preparative HPLC to afford Example 71 as a white solid.

MS obsd. (ESI⁺) [(M+H)⁺]: 447.0. ¹H NMR (400 MHz, DMSO-d6) δ ppm: 9.45 (s, 1H), 8.26 (d, J=2.0 Hz, 1H), 7.98 (d, J=8.8 Hz, 1H), 7.55 (dd, J=8.8, 2.0 Hz, 1H), 7.27-7.31 (m, 2H), 3.36 (d, J=7.2 Hz, 1H), 3.21-3.26 (m, 1H), 2.59 (s, 6H), 0.72-0.90 (m, 1H), 0.53-0.56 (m, 2H), 0.32-0.45 (m, 1H), 0.24-0.28 (m, 1H).

### Example 72

### N-[3-(6-chloro-1,3-benzothiazol-2-yl)-1-bicyclo[1.1.1]pentanyl]-5-methylsulfinyl-furan-2-carboxamide

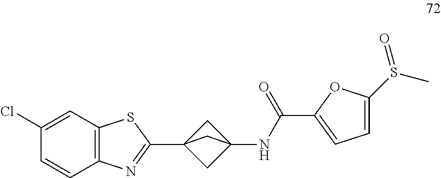 [SMILES: CS(=O)c1ccc(C(=O)NC23CC(c4nc5ccc(Cl)cc5s4)(C2)C3)o1]

The title compound was prepared in analogy to the procedure described for the preparation of Example 71, by using 5-methylsulfanylfuran-2-carboxylic acid (Int-35) instead of 5-(cyclopropylmethylsulfanyl)furan-2-carboxylic acid (Int-50). The product was purified by preparative HPLC to afford Example 72 as a white solid.

MS obsd. (ESI⁺) [(M+H)⁺]: 407.0. ¹H NMR (400 MHz, CD3OD) δ 8.02 (s, 1H), 7.90 (d, J=8.4 Hz, 1H), 7.51 (d, J=8.4 Hz, 1H), 7.21-7.25 (m, 2H), 3.10 (s, 3H), 2.68 (s, 6H).

### Example 73

### N-[3-(6-chloro-1,3-benzothiazol-2-yl)-1-bicyclo[1.1.1]pentanyl]-5-cyclopropylsulfinyl-furan-2-carboxamide

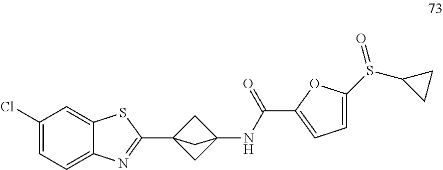 [SMILES: O=C(NC12CC(c3nc4ccc(Cl)cc4s3)(C1)C2)c1ccc(S(=O)C2CC2)o1]

The title compound was prepared in analogy to the procedure described for the preparation of Example 71, by using 5-cyclopropylsulfanylfuran-2-carboxylic acid (Int-49) instead of 5-(cyclopropylmethylsulfanyl)furan-2-carboxylic acid (Int-50). The product was purified by preparative HPLC to afford Example 73 as a white solid.

MS obsd. (ESI⁺) [(M+H)⁺]: 433.1. ¹H NMR (400 MHz, CD₃OD) δ ppm: 8.02 (d, J=1.8 Hz, 1H), 7.90 (d, J=8.6 Hz, 1H), 7.51 (dd, J=8.6, 2.0 Hz, 1H), 7.25 (d, J=3.6 Hz, 1H), 7.20 (d, J=3.6 Hz, 1H), 2.86-2.95 (m, 1H), 2.67 (s, 6H), 1.20-1.35 (m, 2H), 1.10-1.14 (m, 1H), 0.91-1.00 (m, 1H).

### Example 74 and 75

### N-(3-(5-chlorobenzo[d]thiazol-2-yl)bicyclo[1.1.1]pentan-1-yl)-5-(dichloro(methylsulfonyl)methyl)furan-2-carboxamide

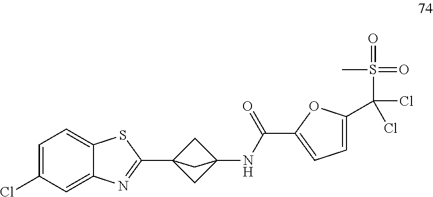 [SMILES: CS(=O)(=O)C(Cl)(Cl)c1ccc(C(=O)NC23CC(c4nc5cc(Cl)ccc5s4)(C2)C3)o1]

### 5-(chloro(methylsulfonyl)methyl)-N-(3-(5-chlorobenzo[d]thiazol-2-yl)bicyclo[1.1.1]pentan-1-yl)furan-2-carboxamide

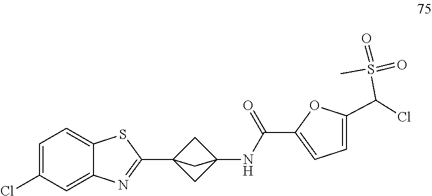 [SMILES: CS(=O)(=O)C(Cl)c1ccc(C(=O)NC23CC(c4nc5cc(Cl)ccc5s4)(C2)C3)o1]

The title compounds were prepared according to the following scheme:

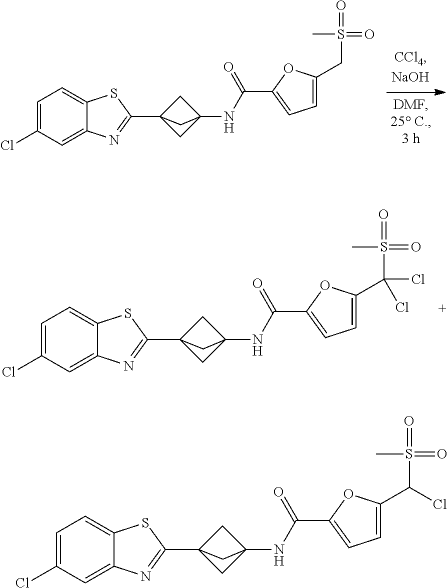 [SMILES: CS(=O)(=O)Cc1ccc(C(=O)NC23CC(c4nc5cc(Cl)ccc5s4)(C2)C3)o1 ; CS(=O)(=O)C(Cl)(Cl)c1ccc(C(=O)NC23CC(c4nc5cc(Cl)ccc5s4)(C2)C3)o1 ; CS(=O)(=O)C(Cl)c1ccc(C(=O)NC23CC(c4nc5cc(Cl)ccc5s4)(C2)C3)o1]

To a mixture of CCl₄ (106 mg, 66.3 μl, 687 μmol) and NaOH (34.3 mg, 858 μmol) in DMF (6 ml) was added N-(3-(5-chlorobenzo[d]thiazol-2-yl)bicyclo[1.1.1]pentan-1-yl)-5-((methylsulfonyl)methyl)furan-2-carboxamide (300 mg, 687 μmol, Example 42) in one portion at 0° C. Then the reaction was stirred at the same temperature for 3 h. The reaction solution was poured into 50 ml of saturated aqueous NH₄Cl. The resulting mixture was extracted with DCM (15 ml×4). The organic layers were washed with brine (30 ml), dried over Na₂SO₄ and concentrated in vacuum. The residue purified by prep-HPLC to give N-(3-(5-chlorobenzo[d]thiazol-2-yl)bicyclo[1.1.1]pentan-1-yl)-5-(dichloro(methylsulfonyl)methyl)furan-2-carboxamide (47 mg) (Example 74) as a white solid and 5-(chloro(methylsulfonyl)methyl)-N-(3-(5-chlorobenzo[d]thiazol-2-yl)bicyclo[1.1.1]pentan-1-yl)furan-2-carboxamide (17 mg) (Example 75) as a white solid.

Example 74: MS obsd. (ESI⁺) [(M+H)⁺]: 505.0. ¹H NMR (DMSO-d₆, 400 MHz): δ (ppm) 9.34 (s, 1H), 8.13 (d, J=8.7 Hz, 1H), 8.08 (d, J=2.0 Hz, 1H), 7.50 (dd, J=8.7, 2.1 Hz, 1H), 7.31 (d, J=3.7 Hz, 1H), 7.22 (d, J=3.8 Hz, 1H), 3.45 (s, 3H), 2.58 (s, 6H).

Example 75: MS obsd. (ESI⁺) [(M+H)⁺]: 470.9. ¹H NMR (DMSO-d₆, 400 MHz): δ (ppm) 9.25 (s, 1H), 8.13 (d, J=8.7 Hz, 1H), 8.08 (d, J=2.0 Hz, 1H), 7.50 (dd, J=8.6, 2.1 Hz, 1H), 7.24 (d, J=3.5 Hz, 1H), 6.93 (d, J=3.5 Hz, 1H), 6.88 (s, 1H), 3.25 (s, 3H), 2.57 (s, 6H).

### Example 76

### N-[3-(6-chloro-1,3-benzothiazol-2-yl)-1-bicyclo[1.1.1]pentanyl]-3-(1-methylsulfonylcyclopropyl)-1,2,4-oxadiazole-5-carboxamide

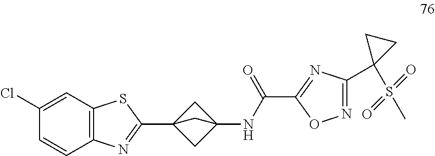 [SMILES: CS(=O)(=O)C1(c2noc(C(=O)NC34CC(c5nc6ccc(Cl)cc6s5)(C3)C4)n2)CC1]

The title compound was prepared according to the following scheme:

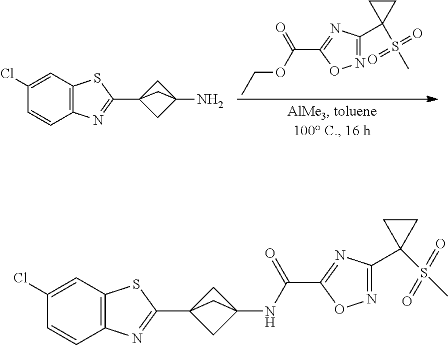 [SMILES: NC12CC(c3nc4ccc(Cl)cc4s3)(C1)C2 ; CCOC(=O)c1nc(C2(S(C)(=O)=O)CC2)no1 ; CS(=O)(=O)C1(c2noc(C(=O)NC34CC(c5nc6ccc(Cl)cc6s5)(C3)C4)n2)CC1]

To a solution of 1-(6-chloro-1,3-benzothiazol-2-yl)bicyclo[1.1.1]pentan-3-amine (125 mg, 0.500 mmol, Int-7) and ethyl 3-(1-methylsulfonylcyclopropyl)-1,2,4-oxadiazole-5-carboxylate (100 mg, 0.380 mmol) (Int-53) in toluene (10 mL) was added trimethylaluminium (0.58 mL, 0.580 mmol, 1.0 M in hexane) at 25° C. under N₂ atmosphere. The resulting mixture was heated to 100° C. and stirred for 16 h. After cooled to room temperature, the resulting solution was concentrated in vacuum. The residue was purified by pre-HPLC to give N-[1-(6-chloro-1,3-benzothiazol-2-yl)-3-bicyclo[1.1.1]pentanyl]-3-(1-methylsulfonylcyclopropyl)-1,2,4-oxadiazole-5-carboxamide (25.9 mg) (Example 76) as a white solid.

MS obsd. (ESI⁺) [(M+H)⁺]: 465.1. ¹H NMR (400 MHz, CDCl3) δ ppm: 7.92 (d, J=8.6 Hz, 1H), 7.84 (s, 1H), 7.64 (s, 1H), 7.42-7.47 (m, 1H), 3.24 (s, 3H), 2.74 (s, 6H), 1.99-2.06 (m, 2H), 1.68-1.74 (m, 2H).

### Example 77

### N-[3-(6-bromo-1,3-benzothiazol-2-yl)-1-bicyclo[1.1.1]pentanyl]-5-(1-methylsulfonylcyclopropyl)-1,2,4-oxadiazole-3-carboxamide

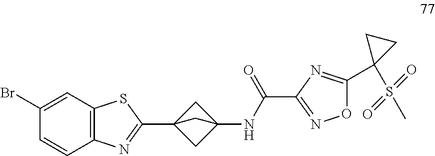 [SMILES: CS(=O)(=O)C1(c2nc(C(=O)NC34CC(c5nc6ccc(Br)cc6s5)(C3)C4)no2)CC1]

The title compound was prepared in analogy to the procedure described for the preparation of Example 76, by using 3-(6-bromo-1,3-benzothiazol-2-yl)bicyclo[1.1.1]pentan-1-amine (Int-1) instead of 1-(6-chloro-1,3-benzothiazol-2-yl)bicyclo[1.1.1]pentan-3-amine (Int-7) and methyl 5-(1-methylsulfonylcyclopropyl)-1,2,4-oxadiazole-3-carboxylate (Int-54) instead of ethyl 3-(1-methylsulfonylcyclopropyl)-1,2,4-oxadiazole-5-carboxylate (Int-53). The product was purified by preparative HPLC to afford Example 77 as a white solid.

MS obsd. (ESI⁺) [(M+H)⁺]: 509.0. ¹H NMR (400 MHz, CDCl3) δ ppm: 7.92 (s, 1H), 7.79 (d, J=8.8 Hz, 1H), 7.48-7.54 (m, 1H), 7.41 (s, 1H), 3.25 (s, 3H), 2.67 (s, 6H), 2.02-2.08 (m, 2H), 1.81-1.85 (m, 2H).

### Example 78

### N-[3-(5-bromo-1,3-benzothiazol-2-yl)-1-bicyclo[1.1.1]pentanyl]-5-(1-methylsulfonylcyclopropyl)-1,2,4-oxadiazole-3-carboxamide

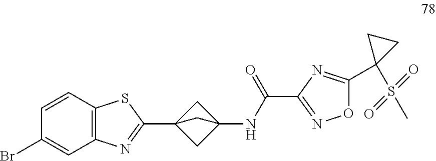 [SMILES: CS(=O)(=O)C1(c2nc(C(=O)NC34CC(c5nc6cc(Br)ccc6s5)(C3)C4)no2)CC1]

The title compound was prepared in analogy to the procedure described for the preparation of Example 76, by using 3-(5-bromo-1,3-benzothiazol-2-yl)bicyclo[1.1.1]pentan-1-amine (Int-2) instead of 1-(6-chloro-1,3-benzothiazol-2-yl)bicyclo[1.1.1]pentan-3-amine (Int-7) and methyl 5-(1-methylsulfonylcyclopropyl)-1,2,4-oxadiazole-3-carboxylate (Int-54) instead of ethyl 3-(1-methylsulfonylcyclopropyl)-1,2,4-oxadiazole-5-carboxylate (Int-53). The product was purified by preparative HPLC to afford Example 78 as a white solid.

MS obsd. (ESI⁺) [(M+H)⁺]: 509.0. ¹H NMR (400 MHz, CDCl3) δ ppm: 8.16 (d, J=1.8 Hz, 1H), 7.72 (d, J=8.6 Hz, 1H), 7.50 (dd, J=8.6, 1.8 Hz, 1H), 7.37 (s, 1H), 3.32 (s, 3H), 2.74 (s, 6H), 2.12-2.16 (m, 2H), 1.88-1.90 (m, 2H).

### Example 79

### 5-(1-methylsulfonylcyclopropyl)-N-[3-(6-morpholino-1,3-benzothiazol-2-yl)-1-bicyclo[1.1.1]pentanyl]furan-2-carboxamide

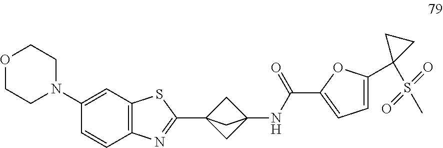 [SMILES: CS(=O)(=O)C1(c2ccc(C(=O)NC34CC(c5nc6ccc(N7CCOCC7)cc6s5)(C3)C4)o2)CC1]

The title compound was prepared according to the following scheme:

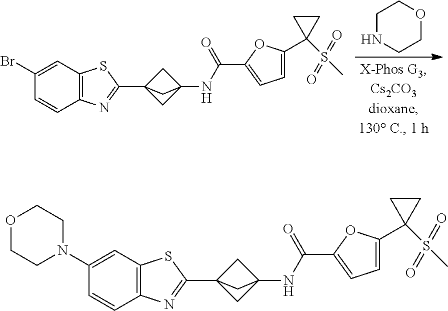 [SMILES: CS(=O)(=O)C1(c2ccc(C(=O)NC34CC(c5nc6ccc(Br)cc6s5)(C3)C4)o2)CC1 ; C1COCCN1 ; CS(=O)(=O)C1(c2ccc(C(=O)NC34CC(c5nc6ccc(N7CCOCC7)cc6s5)(C3)C4)o2)CC1]

A mixture of N-[1-(6-bromo-1,3-benzothiazol-2-yl)-3-bicyclo[1.1.1]pentanyl]-5-(1-methylsulfonylcyclopropyl)furan-2-carboxamide (50 mg, 0.10 mmol, Example 2), morpholine (13 mg, 0.15 mmol), XPhos G₃ (8.33 mg, 0.010 mmol), Cesium carbonate (96 mg, 0.30 mmol) in 1,4-dioxane (2 mL) was stirred at 100° C. for 2 h under nitrogen atmosphere. After cooled to 25° C., the resulting solution was diluted with EtOAc (30 mL) and filtered through a celite pad. The filtrate was washed with brine (20 mL×3), dried over Na₂SO₄ and concentrated in vacuum. The residue was purified by prep-HPLC to give 5-(1-methylsulfonylcyclopropyl)-N-[1-(6-morpholino-1,3-benzothiazol-2-yl)-3-bicyclo[1.1.1]pentanyl]furan-2-carboxamide (21.2 mg) (Example 79) as a white solid.

MS obsd. (ESI⁺) [(M+H)⁺]: 514.2. ¹H NMR (400 MHz, CD3OD) d ppm: 7.79 (d, J=8.8 Hz, 1H), 7.45 (d, J=2.4 Hz, 1H), 7.23 (dd, J=8.8, 2.4 Hz, 1H), 7.12 (d, J=3.6 Hz, 1H), 6.78 (d, J=3.6 Hz, 1H), 3.81-3.89 (m, 4H), 3.16-3.25 (m, 4H), 3.02 (s, 3H), 2.63 (s, 6H), 1.79 (q, J=4.8 Hz, 2H), 1.56 (q, J=5.2 Hz, 2H).

### Example 80

### 5-(1-methylsulfonylcyclopropyl)-N-[3-(5-morpholino-1,3-benzothiazol-2-yl)-1-bicyclo[1.1.1]pentanyl]furan-2-carboxamide

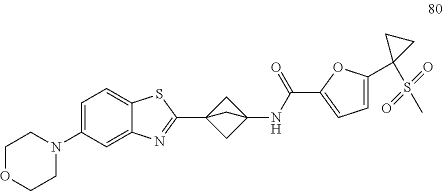 [SMILES: CS(=O)(=O)C1(c2ccc(C(=O)NC34CC(c5nc6cc(N7CCOCC7)ccc6s5)(C3)C4)o2)CC1]

The title compound was prepared in analogy to the procedure described for the preparation of Example 79, by using N-(3-(5-bromobenzo[d]thiazol-2-yl)bicyclo[1.1.1]pentan-1-yl)-5-(1-(methylsulfonyl)cyclopropyl)furan-2-carboxamide (Example 6) instead of N-[1-(6-bromo-1,3-benzothiazol-2-yl)-3-bicyclo[1.1.1]pentanyl]-5-(1-methylsulfonylcyclopropyl)furan-2-carboxamide (Example 2). The product was purified by preparative HPLC to afford Example 80 as a white solid.

MS obsd. (ESI⁺) [(M+H)⁺]: 514.0. ¹H NMR (400 MHz, CD₃OD) δ ppm: 7.81 (d, J=8.6 Hz, 1H), 7.45 (s, 1H), 7.14-7.22 (m, 2H), 6.80 (br. s, 1H), 3.85-3.42 (m, 4H), 3.20-3.25 (m, 4H), 3.03 (s, 3H), 2.65 (s, 6H), 1.80 (d, J=5.0 Hz, 2H), 1.57 (d, J=5.0 Hz, 2H).

### Example 81

### N-[3-[6-(2-methoxyethoxy)-1,3-benzothiazol-2-yl]-1-bicyclo[1.1.1]pentanyl]-5-(1-methylsulfonylcyclopropyl)furan-2-carboxamide

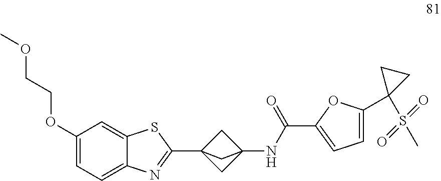 [SMILES: COCCOc1ccc2nc(C34CC(NC(=O)c5ccc(C6(S(C)(=O)=O)CC6)o5)(C3)C4)sc2c1]

The title compound was prepared according to the following scheme:

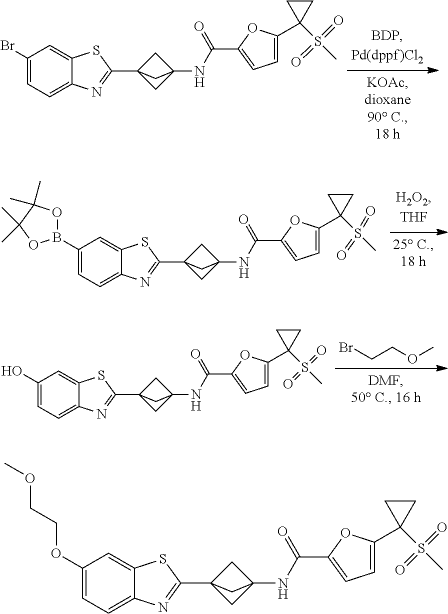

### Step 1: Preparation of 5-(1-methylsulfonylcyclopropyl)-N-[3-[6-(4,4,5,5-tetramethyl-1,3,2-dioxaborolan-2-yl)-1,3-benzothiazol-2-yl]-1-bicyclo[1.1.1]pentanyl]furan-2-carboxamide

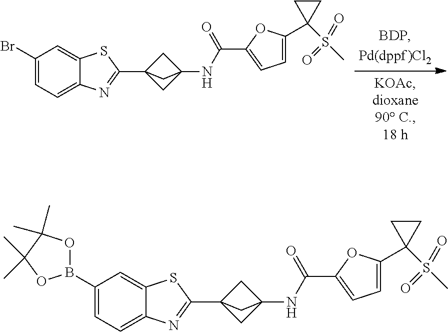

To a solution of N-[1-(6-bromo-1,3-benzothiazol-2-yl)-3-bicyclo[1.1.1]pentanyl]-5-(1-methylsulfonylcyclopropyl)furan-2-carboxamide (500 mg, 0.99 mmol, Example 2) in 1,4-dioxane (15 mL) were added bis(pinacolato)diboron (299 mg, 1.18 mmol), potassium acetate (193 mg, 1.97 mmol) and 1,1′-bis(diphenylphosphino)ferrocene]dichloropalladium (72 mg, 0.10 mmol) under nitrogen atmosphere. The reaction mixture was stirred at 90° C. for 18 h under nitrogen atmosphere. After cooled to room temperature, the resulting mixture was diluted with EtOAc (60 mL) and washed with water (15 mL×2) and brine (15 mL). The organic layer was dried over Na₂SO₄ and concentrated in vacuum. The residue was purified by silica gel column eluted with DCM/MeOH=20/1 to give 5-(1-methylsulfonylcyclopropyl)-N-[3-[6-(4,4,5,5-tetramethyl-1,3,2-dioxaborolan-2-yl)-1,3-benzothiazol-2-yl]-1-bicyclo[1.1.1]pentanyl]furan-2-carboxamide (444 mg) as yellow oil.

MS obsd. (ESI⁺) [(M+H)⁺]: 555.1.

### Step 2: Preparation of N-[3-(6-hydroxy-1,3-benzothiazol-2-yl)-1-bicyclo[1.1.1]pentanyl]-5-(1-methylsulfonylcyclopropyl)furan-2-carboxamide

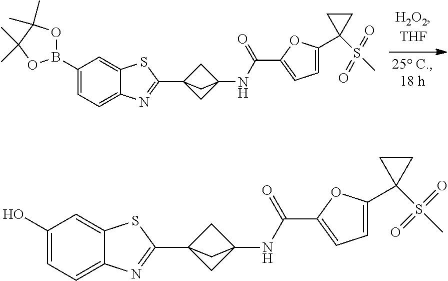

A mixture of 5-(1-methylsulfonylcyclopropyl)-N-[3-[6-(4,4,5,5-tetramethyl-1,3,2-dioxaborolan-2-yl)-1,3-benzothiazol-2-yl]-1-bicyclo[1.1.1]pentanyl]furan-2-carboxamide (307 mg, 0.55 mmol) and hydrogen peroxide (0.06 mL, 0.66 mmol) in THF (10 ml) was stirred at 25° C. for 18 h. The reaction solution was diluted with EtOAc (50 mL). The resulting solution was washed with saturated aqueous Na₂S₂O₃ (10 mL×2) and brine (15 mL). The organic layer was dried over Na₂SO₄ and concentrated in vacuum. The residue was purified by prep-HPLC to give N-[3-(6-hydroxy-1,3-benzothiazol-2-yl)-1-bicyclo[1.1.1]pentanyl]-5-(1-methylsulfonylcyclopropyl)furan-2-carboxamide (210 mg) as a white solid.

MS obsd. (ESI⁺) [(M+H)⁺]: 445.1.

### Step 3: Preparation of N-[3-[6-(2-methoxyethoxy)-1,3-benzothiazol-2-yl]-1-bicyclo[1.1.1]pentanyl]-5-(1-methylsulfonylcyclopropyl)furan-2-carboxamide

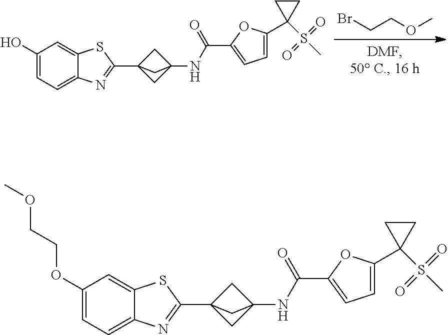

To a solution of N-[3-(6-hydroxy-1,3-benzothiazol-2-yl)-1-bicyclo[1.1.1]pentanyl]-5-(1-methylsulfonylcyclopropyl)furan-2-carboxamide (100 mg, 0.22 mmol) in DMF (10 mL) were added potassium carbonate (62 mg, 0.45 mmol) and 1-bromo-2-methoxyethane (31 mL, 0.22 mmol). The reaction mixture was stirred at 50° C. for 16 h. After cooled to room temperature, the resulting solution was diluted with EtOAc (100 mL×3) and washed with water (100 mL×2) and brine (150 mL). The organic layer was dried over Na₂SO₄ and concentrated in vacuum. The residue was purified by prep-HPLC to give N-[3-[6-(2-methoxyethoxy)-1,3-benzothiazol-2-yl]-1-bicyclo[1.1.1]pentanyl]-5-(1-methylsulfonylcyclopropyl)furan-2-carboxamide (2.5 mg) as a white solid.

MS obsd. (ESI⁺) [(M+H)⁺]: 503.0. ¹H NMR (400 MHz, CD3OD) δ ppm: 7.81 (d, J=8.8 Hz, 1H), 7.51 (d, J=2.4 Hz, 1H), 7.11-7.17 (m, 2H), 6.78 (d, J=3.6 Hz, 1H), 4.16-4.22 (m, 2H), 3.75-3.80 (m, 2H), 3.43 (s, 3H), 3.02 (s, 3H), 2.64 (s, 6H), 1.79 (q, J=5.0 Hz, 2H), 1.56 (q, J=5.0 Hz, 2H).

### Example 82

### N-[3-(6-chloro-1,3-benzothiazol-2-yl)-1-bicyclo[1.1.1]pentanyl]-3-(1-methylsulfonylethyl)-1H-pyrazole-5-carboxamide

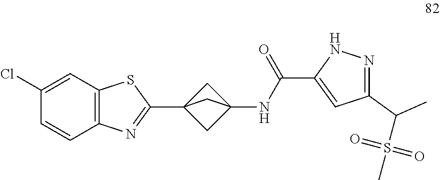 [SMILES: CC(c1cc(C(=O)NC23CC(c4nc5ccc(Cl)cc5s4)(C2)C3)[nH]n1)S(C)(=O)=O]

The title compound was prepared according to the following scheme:

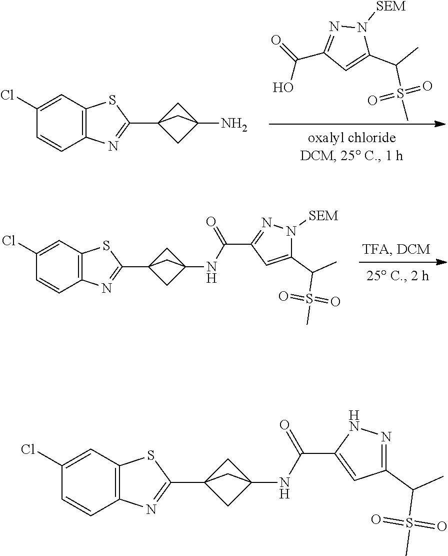 [SMILES: NC12CC(c3nc4ccc(Cl)cc4s3)(C1)C2 ; CC(c1cc(C(=O)O)nn1COCC[Si](C)(C)C)S(C)(=O)=O ; CC(c1cc(C(=O)NC23CC(c4nc5ccc(Cl)cc5s4)(C2)C3)nn1COCC[Si](C)(C)C)S(C)(=O)=O ; CC(c1cc(C(=O)NC23CC(c4nc5ccc(Cl)cc5s4)(C2)C3)[nH]n1)S(C)(=O)=O]

### Step 1: Preparation of N-[3-(6-chloro-1,3-benzothiazol-2-yl)-1-bicyclo[1.1.1]pentanyl]-5-(1-methylsulfonylethyl)-1-(2-trimethylsilylethoxymethyl)pyrazole-3-carboxamide

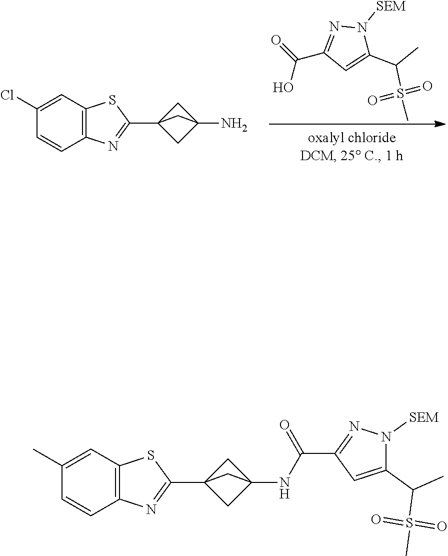 [SMILES: NC12CC(c3nc4ccc(Cl)cc4s3)(C1)C2 ; CC(c1cc(C(=O)O)nn1COCC[Si](C)(C)C)S(C)(=O)=O ; Cc1ccc2nc(C34CC(NC(=O)c5cc(C(C)S(C)(=O)=O)n(COCC[Si](C)(C)C)n5)(C3)C4)sc2c1]

To a solution of 5-(1-methylsulfonylethyl)-1-(2-trimethylsilylethoxymethyl)pyrazole-3-carboxylic acid (50 mg, 0.140 mmol, Int-55) in DCM (5 mL) was added oxalyl chloride (20 mg, 0.160 mmol). After stirring at 25° C. for 30 min, the reaction mixture was concentrated in vacuum. The residue was dissolved in DCM (5 mL) and a solution of 1-(6-chloro-1,3-benzothiazol-2-yl)bicyclo[1.1.1]pentan-3-amine (35.98 mg, 0.140 mmol, Int-7) in DCM (5 mL) was added slowly. The reaction was stirred at room temperature for 1 h. The resulted solution was concentrated in vacuum to give N-[3-(6-chloro-1,3-benzothiazol-2-yl)-1-bicyclo[1.1.1]pentanyl]-5-(1-methylsulfonylethyl)-1-(2-trimethylsilylethoxymethyl)pyrazole-3-carboxamide (50 mg) as a grey solid.

MS obsd. (ESI⁺) [(M+H)⁺]: 581.0.

### Step 2: Preparation of N-[3-(6-chloro-1,3-benzothiazol-2-yl)-1-bicyclo[1.1.1]pentanyl]-5-(1-methylsulfonylethyl)-1H-pyrazole-3-carboxamide

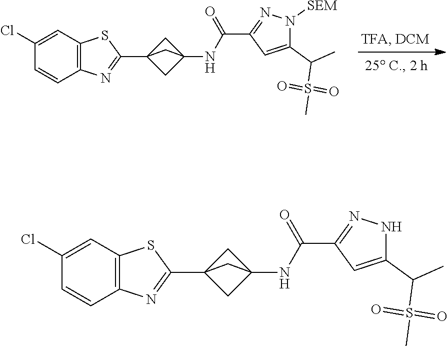 [SMILES: CC(c1cc(C(=O)NC23CC(c4nc5ccc(Cl)cc5s4)(C2)C3)nn1COCC[Si](C)(C)C)S(C)(=O)=O ; CC(c1cc(C(=O)NC23CC(c4nc5ccc(Cl)cc5s4)(C2)C3)n[nH]1)S(C)(=O)=O]

To a solution of N-[1-(6-chloro-1,3-benzothiazol-2-yl)-3-bicyclo[1.1.1]pentanyl]-2-(3,3-dimethylbutoxymethyl)-5-(1-methylsulfonylethyl)pyrazole-3-carboxamide (50 mg, 0.090 mmol) in DCM (2 mL) was added TFA (2.0 mL, 26.93 mmol) at 25° C. The reaction was stirred at room temperature for 2 h and then concentrated in vacuum. The residue was purified by prep-HPLC to give N-[3-(6-chloro-1,3-benzothiazol-2-yl)-1-bicyclo[1.1.1]pentanyl]-5-(1-methylsulfonylethyl)-1H-pyrazole-3-carboxamide (2.4 mg) (Example 82) as a white solid.

MS obsd. (ESI⁺) [(M+H)⁺]: 451.0. ¹H NMR (400 MHz, DMSO-d6) δ ppm: 9.34 (s, 1H), 9.00 (s, 1H), 8.31 (s, 1H), 8.03 (d, J=8.8 Hz, 1H), 7.61 (d, J=8.8 Hz, 1H), 7.03 (s, 1H), 3.35 (q, J=6.4 Hz, 1H), 2.91 (s, 3H), 2.62 (s, 6H), 1.66 (d, J=6.4 Hz, 3H).

### Example 83

### 5-(butanoylsulfamoyl)-N-[3-(6-chloro-1,3-benzothiazol-2-yl)-1-bicyclo[1.1.1]pentanyl]furan-2-carboxamide

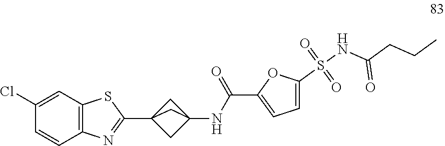 [SMILES: CCCC(=O)NS(=O)(=O)c1ccc(C(=O)NC23CC(c4nc5ccc(Cl)cc5s4)(C2)C3)o1]

The title compound was prepared according to the following scheme:

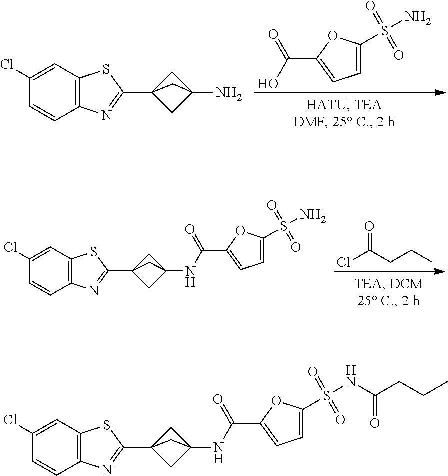

### Step 1: Preparation of N-[3-(6-chloro-1,3-benzothiazol-2-yl)-1-bicyclo[1.1.1]pentanyl]-5-sulfamoyl-furan-2-carboxamide

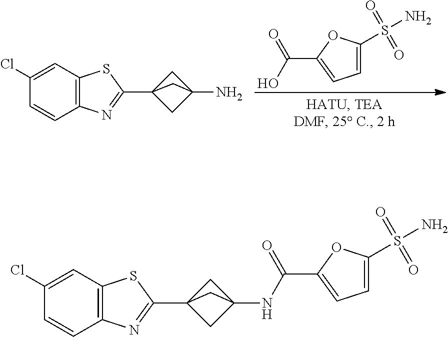 [SMILES: NC12CC(c3nc4ccc(Cl)cc4s3)(C1)C2 ; NS(=O)(=O)c1ccc(C(=O)O)o1 ; NS(=O)(=O)c1ccc(C(=O)NC23CC(c4nc5ccc(Cl)cc5s4)(C2)C3)o1]

To a solution of 1-(6-chloro-1,3-benzothiazol-2-yl)bicyclo[1.1.1]pentan-3-amine (656 mg, 2.62 mmol, Int-7) and 5-sulfamoylfuran-2-carboxylic acid (500 mg, 2.62 mmol) in DMF (20 mL) were added HATU (995 mg, 2.62 mmol) and TEA (265 mg, 2.62 mmol). The reaction was stirred at 25° C. for 2 h. The reaction mixture was diluted with DCM (40 ML) and washed with water (20 mL×3). The organic phase was dried over Na₂SO₄ and concentrated in vacuum. The residue was purified by prep-HPLC to give N-[3-(6-chloro-1,3-benzothiazol-2-yl)-1-bicyclo[1.1.1]pentanyl]-5-sulfamoyl-furan-2-carboxamide (108 mg) as a white solid.

MS obsd. (ESI⁺) [(M+H)⁺]: 424.0. ¹H NMR (400 MHz, CD₃OD) δ ppm: 8.02 (d, J=2.0 Hz, 1H), 7.90 (d, J=8.8 Hz, 1H), 7.51 (dd, J=8.8, 2.0 Hz, 1H), 7.18 (d, J=3.6 Hz, 1H), 7.04 (d, J=3.6 Hz, 1H), 2.66 (s, 6H).

### Step 2: Preparation of 5-(butanoylsulfamoyl)-N-[3-(6-chloro-1,3-benzothiazol-2-yl)-1-bicyclo[1.1.1]pentanyl]furan-2-carboxamide

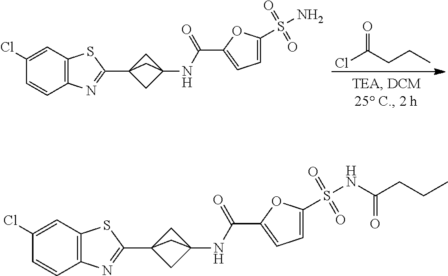

To a solution of butyryl chloride (0.06 mL, 0.54 mmol) in DCM (3 mL) were added TEA (0.15 mL, 1.09 mmol) and N-[3-(6-chloro-1,3-benzothiazol-2-yl)-1-bicyclo[1.1.1]pentanyl]-5-sulfamoyl-furan-2-carboxamide (100 mg, 0.54 mmol). The reaction was stirred at 25° C. for 2 h. The resulted solution was diluted with DCM (20 mL) and washed water (15 mL) and brine (15 mL). The organic phase was dried over Na₂SO₄ and concentrated in vacuum. The residue was purified by prep-HPLC to give 5-(butanoylsulfamoyl)-N-[3-(6-chloro-1,3-benzothiazol-2-yl)-1-bicyclo[1.1.1]pentanyl]furan-2-carboxamide (17.5 mg) (Example 83) as a white solid.

MS obsd. (ESI⁺) [(M+H)⁺]: 494.0. ¹H NMR (400 MHz, CD₃OD) δ ppm: 8.02 (s, 1H), 7.90 (d, J=8.8 Hz, 1H), 7.50 (d, J=8.8 Hz, 1H), 7.16 (d, J=3.6 Hz, 1H), 7.11 (d, J=3.6 Hz, 1H), 2.66 (s, 6H), 2.20 (t, J=7.4 Hz, 2H), 1.53-1.65 (m, 2H), 0.89 (t, J=7.4 Hz, 3H).

### BIOLOGICAL EXAMPLES

### Example 84

### PHH Natural Infection Assay

Detailed procedures regarding primary human hepatocyte (PHH) HBV natural infection assay are described as below. One tube of frozen PHH (10 million cells) is thawed in 37° C. water bath and then transferred to 20 mL of PHH thawing medium (Sigma, InVitroGRO HT Medium, Cat. S03319) with gently mixing. The cells were then centrifuged at 80 g/min for 5 min, the supernatant was discarded and the tube was refilled with 25 mL of PHH plating medium (Sigma, InVitroGRO CP Medium, Cat. S03317). The tube was shaken very gently to re-suspend all cells, and then 50 μl of cells were transferred to each well 384-well collagen I coated plate with appropriate liquid handling equipment, e.g. Integra VIAFLO384 or Agilent Bravo. The cells were then cultured for 24 hours in a cell incubator. For HBV infection, after PHH attachment on the culture plate, the plating medium was removed and replenished with PHH culture medium containing HBV virus. The PHH culture medium was prepared with Dulbecco's Modified Eagle Medium (DMEM)/F12 (1:1 in volume ratio) containing 10% fetal bovine serum (Gibco, Cat.10099141), 5 ng/mL human epidermal growth factor (Gibco, Cat.PHG0311L), 20 ng/mL dexamethasone (Sigma, Cat.D4902-100 mg), 250 ng/mL human recombinant insulin (Gibco, Cat.41400045) and 100 U/mL penicillin. HBV virus at 200 genome equivalent (GE) per cell with 4% PEG8000 (Sigma, Cat.P1458) containing culture medium were added to the PHH culture medium for infection. The cells were then cultured for 24 hours in cell incubator. Then the cell culture supernatant was removed. The HBV-infected PHH were cultured with sandwich culture method with PHH culture medium containing 1% DMSO and 0.25 mg/mL matrix gel for 72 hours. The supernatant was then refreshed with PHH culture medium containing different concentrations of testing compounds for two times with 72-hour interval. At the end of treatment, the supernatant was collected for viral markers measurements, including HBsAg, HBeAg, HBV DNA and cytotoxicity. HBsAg and HBeAg were detected using alphalisa method using their specific antibodies. For HBV DNA detection, HBV DNA Quantitative Fluorescence Diagnostic Kit (Sansure Biotech Inc.) was used following the manufacture's protocol. Cytotoxicity was determined using Cell Counting Kit-8 (CCK8, Dojindo Molecular Technologies, Inc.).

The compounds of the present invention were tested for their capacity to inhibit HBsAg and HBeAg as described herein. The Examples were tested in the above assay and found to have IC₅₀ below 10 μM. Results of PHH assay are given in Table 3.

TABLE 3
Activity data of compounds of this invention

| Example No. | HBsAg IC₅₀ (μM) | HBeAg IC₅₀ (μM) | CC₅₀ (μM) |
|---|---|---|---|
| Example 2 | 0.17 | 0.11 | 5.85 |
| Example 4 | 0.07 | 0.05 | 4.22 |
| Example 6 | 0.17 | 0.10 | 6.03 |
| Example 7 | 0.10 | 0.06 | 8.52 |
| Example 8 | 0.79 | 0.72 | 12.5 |
| Example 10 | 0.37 | 0.27 | 11.0 |
| Example 11 | 0.48 | 0.36 | 16.3 |
| Example 13 | 1.02 | 0.80 | >50.0 |
| Example 14 | 0.20 | 0.15 | >10.0 |
| Example 15 | 0.21 | 0.16 | 6.33 |
| Example 16 | 0.29 | 0.24 | 3.76 |
| Example 17 | 0.17 | 0.15 | 14.6 |
| Example 22 | 0.72 | 0.47 | 7.55 |
| Example 27 | 0.84 | 0.65 | 8.55 |
| Example 31 | 0.74 | 0.63 | 5.71 |
| Example 32 | 0.43 | 0.33 | 2.95 |
| Example 34 | 0.44 | 0.39 | 7.01 |
| Example 43 | 0.50 | 0.48 | 22.8 |
| Example 44 | 0.23 | 0.16 | 20.4 |
| Example 45 | 0.39 | 0.25 | 9.94 |
| Example 46 | 0.37 | 0.23 | 15.3 |
| Example 47 | 0.32 | 0.22 | 6.62 |
| Example 50 | 0.10 | 0.08 | 1.64 |
| Example 56a | 0.23 | 0.23 | 15.0 |
| Example 56b | 0.19 | 0.16 | 14.7 |
| Example 57a | 0.36 | 0.36 | 22.3 |
| Example 57b | 0.49 | 0.38 | 17.4 |
| Example 58 | 0.34 | 0.24 | 6.06 |
| Example 60 | 0.122 | 0.09 | 4.82 |
| Example 61 | 0.11 | 0.08 | 2.09 |
| Example 62 | 0.07 | 0.04 | 3.83 |
| Example 63 | 0.11 | 0.08 | 5.06 |
| Example 67 | 0.40 | 0.32 | 7.25 |
| Example 75 | 0.55 | 0.43 | 16.8 |
| Example 76 | 0.70 | 0.40 | 4.27 |
| Example 77 | 0.86 | 0.62 | 6.49 |

1. A compound of formula (I), 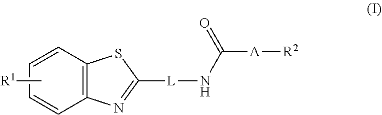
wherein
R¹ is hydrogen, halogen, C₂₋₆alkynyl, cyano, morpholinyl, or C₁₋₆alkoxyC₁₋₆alkoxy;
L is a C₅₋₁₂cycloalkyl, wherein L is a monocyclic ring or a bicyclic ring, and wherein the bicyclic ring is a bridged, spiro or fused ring;
A is a 5 or 6 membered heteroaryl containing one to three heteroatoms independently selected from N, O, and S;
R² is C₁₋₆alkylsulfonylC₁₋₆alkyl, C₁₋₆alkylsulfonylC₃₋₇cycloalkyl, halo(C₁₋₆alkylsulfonyl)C₁₋₆alkyl, carbamoyl(C₁₋₆alkylsulfonyl)C₁₋₆alkyl, C₁₋₆alkylsulfonyl, C₁₋₆alkoxysulfonyl, (C₁₋₆alkoxyC₁₋₆alkyl)sulfonyl, C₃₋₇cycloalkylsulfonyl, C₁₋₆alkylsulfinylC₁₋₆alkyl, C₁₋₆alkylsulfinyl, C₃-7cycloalkylC₁₋₆alkylsulfinyl, C₃₋₇cycloalkylsulfinyl, C₁₋₆alkylsulfonimidoyl, C₃₋₇cycloalkylC₁₋₆alkylsulfonimidoyl, C₃₋₇cycloalkylsulfonimidoyl, C₁₋₆alkylsulfanyl, C₁₋₆alkylsulfanylC₁₋₆alkyl, haloC₁₋₆alkyl, haloC₁₋₆alkylsulfonylC₁₋₆alkyl, C₃₋₇cycloalkylC₁₋₆alkylsulfonyl, or (C₁₋₆alkylcarbonyl)sulfamoyl;
or a pharmaceutically acceptable salt thereof.

2. The compound according to claim 1, wherein
R¹ is halogen or C₂₋₆alkynyl.

3. The compound according to claim 2, wherein
R¹ is fluoro, chloro, bromo, or ethynyl.

4. The compound according to claim 1, wherein
L is 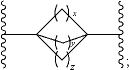
each of x, y, and z is independently an integer of 1, 2, or 3.

5. The compound according to claim 4, wherein
L is

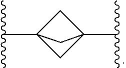 [SMILES: *C12CC(*)(C1)C2]

6. The compound according to claim 1, wherein
A is furanyl, oxadiazolyl, thiadiazolyl, oxazolyl, dihydrothiazolyl, thiazolyl, isothiazolyl, pyrazolyl, triazolyl, or pyridinyl.

7. The compound according to claim 6, wherein
A is furanyl, thiadiazolyl, oxazolyl, or pyrazolyl.

8. The compound according to claim 7, wherein
A is furanyl, 1,2,4-thiadiazolyl, oxazolyl, or pyrazolyl.

9. The compound according to claim 1, wherein
R² is C₁₋₆alkylsulfonylC₃₋₇cycloalkyl, C₁₋₆alkylsulfonylC₁₋₆alkyl, haloC₁₋₆alkylsulfonylC₁₋₆alkyl, or C₁₋₆alkylsulfinylC₁₋₆alkyl.

10. The compound according to claim 9, wherein
R² is methylsulfonylcyclopropyl, 1-(methylsulfonyl)propyl, 2-(methylsulfonyl)propan-2-yl, 1-(methylsulfonyl)ethyl, bromo(methylsulfonyl)methyl, or 2-(methylsulfinyl)propan-2-yl.

11. The compound according to claim 1, wherein
R¹ is halogen or C₂₋₆alkynyl;
L is 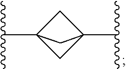 [SMILES: *C12CC(*)(C1)C2]
A is furanyl, thiadiazolyl, oxazolyl, or pyrazolyl; and
R² is C₁₋₆alkylsulfonylC₃₋₇cycloalkyl, C₁₋₆alkylsulfonylC₁₋₆alkyl, haloC₁₋₆alkylsulfonylC₁₋₆alkyl, or C₁₋₆alkylsulfinylC₁₋₆alkyl.

12. The compound according to claim 11, wherein
R¹ is fluoro, chloro, bromo, or ethynyl;
L is 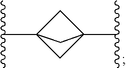 [SMILES: *C12CC(*)(C1)C2]
A is furanyl, 1,2,4-thiadiazolyl, oxazolyl, or pyrazolyl; and
R² is methylsulfonylcyclopropyl, 1-(methylsulfonyl)propyl, 2-(methylsulfonyl)propan-2-yl, 1-(methylsulfonyl)ethyl, bromo(methylsulfonyl)methyl, or 2-(methylsulfinyl)propan-2-yl.

13. A compound selected from:
N-[3-(6-bromo-1,3-benzothiazol-2-yl)-1-bicyclo[1.1.1]pentanyl]-5-(methylsulfonylmethyl)furan-2-carboxamide;
N-[3-(6-bromo-1,3-benzothiazol-2-yl)-1-bicyclo[1.1.1]pentanyl]-5-(1-methylsulfonylcyclopropyl)furan-2-carboxamide;
N-[3-(6-bromo-1,3-benzothiazol-2-yl)-1-bicyclo[1.1.1]pentanyl]-5-(1-methylsulfonylcyclopropyl)-1,3,4-oxadiazole-2-carboxamide;
N-[3-(6-bromo-1,3-benzothiazol-2-yl)-1-bicyclo[1.1.1]pentanyl]-3-(1-methylsulfonylcyclopropyl)-1,2,4-thiadiazole-5-carboxamide;
N-[3-(5-bromo-1,3-benzothiazol-2-yl)-1-bicyclo[1.1.1]pentanyl]-5-(methylsulfonylmethyl)furan-2-carboxamide;
N-(3-(5-bromobenzo[d]thiazol-2-yl)bicyclo[1.1.1]pentan-1-yl)-5-(1-(methylsulfonyl)cyclopropyl)furan-2-carboxamide;
N-[3-(5-bromo-1,3-benzothiazol-2-yl)-1-bicyclo[1.1.1]pentanyl]-3-(1-methylsulfonylcyclopropyl)-1,2,4-thiadiazole-5-carboxamide;
N-[3-(6-cyano-1,3-benzothiazol-2-yl)-1-bicyclo[1.1.1]pentanyl]-5-(1-methylsulfonylcyclopropyl)furan-2-carboxamide;
N-[3-(5-cyano-1,3-benzothiazol-2-yl)-1-bicyclo[1.1.1]pentanyl]-5-(1-methylsulfonylcyclopropyl)furan-2-carboxamide;
N-[3-(6-ethynyl-1,3-benzothiazol-2-yl)-1-bicyclo[1.1.1]pentanyl]-5-(1-methylsulfonylcyclopropyl)furan-2-carboxamide;
N-[3-(6-fluoro-1,3-benzothiazol-2-yl)-1-bicyclo[1.1.1]pentanyl]-5-(1-methylsulfonylcyclopropyl)furan-2-carboxamide;
N-[3-(6-chloro-1,3-benzothiazol-2-yl)-1-bicyclo[1.1.1]pentanyl]-5-cyclopropylsulfonyl-furan-2-carboxamide;
N-[3-(6-chloro-1,3-benzothiazol-2-yl)-1-bicyclo[1.1.1]pentanyl]-5-(methylsulfonylmethyl)furan-2-carboxamide;
N-[3-(6-chloro-1,3-benzothiazol-2-yl)-1-bicyclo[1.1.1]pentanyl]-5-(1-methylsulfonylpropyl)furan-2-carboxamide;
N-(3-(6-chlorobenzo[d]thiazol-2-yl)bicyclo[1.1.1]pentan-1-yl)-5-(1-(methylsulfonyl)cyclopropyl)furan-2-carboxamide;
N-[3-(6-chloro-1,3-benzothiazol-2-yl)-1-bicyclo[1.1.1]pentanyl]-5-(1-methyl-1-methylsulfonyl-ethyl)furan-2-carboxamide;
N-[3-(6-chloro-1,3-benzothiazol-2-yl)-1-bicyclo[1.1.1]pentanyl]-5-(1-methylsulfonylethyl)furan-2-carboxamide;
5-[bromo(methylsulfonyl)methyl]-N-[3-(6-chloro-1,3-benzothiazol-2-yl)-1-bicyclo[1.1.1]pentanyl]furan-2-carboxamide;
5-(2-amino-1-methylsulfonyl-2-oxo-ethyl)-N-[3-(6-chloro-1,3-benzothiazol-2-yl)-1-bicyclo[1.1.1]pentanyl]furan-2-carboxamide;
N-[3-(6-chloro-1,3-benzothiazol-2-yl)-1-bicyclo[1.1.1]pentanyl]-2-(methylsulfonylmethyl)oxazole-5-carboxamide;
N-[3-(6-chloro-1,3-benzothiazol-2-yl)-1-bicyclo[1.1.1]pentanyl]-2-(1-methylsulfonylethyl)oxazole-5-carboxamide;
N-[3-(6-chloro-1,3-benzothiazol-2-yl)-1-bicyclo[1.1.1]pentanyl]-2-(1-methyl-1-methylsulfonyl-ethyl)oxazole-5-carboxamide;
N-[6-(5-chloro-1,3-benzoxazol-2-yl)spiro[3.3]heptan-2-yl]-2-(methylsulfamoylamino)pyridine-4-carboxamide;
N-[3-(6-chloro-1,3-benzothiazol-2-yl)-1-bicyclo[1.1.1]pentanyl]-2-(1-methylsulfonylcyclopropyl)thiazole-5-carboxamide;
N-[3-(6-chloro-1,3-benzothiazol-2-yl)-1-bicyclo[1.1.1]pentanyl]-2-(1-methyl-1-methylsulfonyl-ethyl)thiazole-5-carboxamide;
N-[3-(6-chloro-1,3-benzothiazol-2-yl)-1-bicyclo[1.1.1]pentanyl]-3-(methylsulfonylmethyl)isothiazole-5-carboxamide;
N-[3-(6-chloro-1,3-benzothiazol-2-yl)-1-bicyclo[1.1.1]pentanyl]-3-(1-methyl-1-methylsulfonyl-ethyl)isothiazole-5-carboxamide;
N-[3-(6-chloro-1,3-benzothiazol-2-yl)-1-bicyclo[1.1.1]pentanyl]-5-(1-methyl-1-methylsulfonyl-ethyl)isothiazole-3-carboxamide;
N-[3-(6-chloro-1,3-benzothiazol-2-yl)-1-bicyclo[1.1.1]pentanyl]-5-(1-methylsulfonylcyclopropyl)isothiazole-3-carboxamide;
N-[3-(6-chloro-1,3-benzothiazol-2-yl)-1-bicyclo[1.1.1]pentanyl]-3-(methylsulfonylmethyl)-1,2,4-thiadiazole-5-carboxamide;
N-[3-(6-chloro-1,3-benzothiazol-2-yl)-1-bicyclo[1.1.1]pentanyl]-3-(1-methylsulfonylethyl)-1,2,4-thiadiazole-5-carboxamide;
N-[3-(6-chloro-1,3-benzothiazol-2-yl)-1-bicyclo[1.1.1]pentanyl]-3-(1-methyl-1-methylsulfonyl-ethyl)-1,2,4-thiadiazole-5-carboxamide;
N-[3-(6-chloro-1,3-benzothiazol-2-yl)-1-bicyclo[1.1.1]pentanyl]-1-(methylsulfonylmethyl)pyrazole-3-carboxamide;
N-[3-(6-chloro-1,3-benzothiazol-2-yl)-1-bicyclo[1.1.1]pentanyl]-1-(1-methylsulfonylethyl)pyrazole-3-carboxamide;
N-[3-(6-chloro-1,3-benzothiazol-2-yl)-1-bicyclo[1.1.1]pentanyl]-3-(methylsulfonylmethyl)-1,2,4-oxadiazole-5-carboxamide;
N-[3-(6-chloro-1,3-benzothiazol-2-yl)-1-bicyclo[1.1.1]pentanyl]-5-(1-methylsulfonylcyclopropyl)-1,3,4-oxadiazole-2-carboxamide;
N-[3-(6-chloro-1,3-benzothiazol-2-yl)-1-bicyclo[1.1.1]pentanyl]-2-(methylsulfonylmethyl)triazole-4-carboxamide;
N-[3-(6-chloro-1,3-benzothiazol-2-yl)-1-bicyclo[1.1.1]pentanyl]-2-(trifluoromethyl)pyridine-4-carboxamide;
N-(3-(5-chlorobenzo[d]thiazol-2-yl)bicyclo[1.1.1]pentan-1-yl)-5-(methylthio)furan-2-carboxamide;
N-(3-(5-chlorobenzo[d]thiazol-2-yl)bicyclo[1.1.1]pentan-1-yl)-5-(methylsulfonyl)furan-2-carboxamide;
N-(3-(5-chlorobenzo[d]thiazol-2-yl)bicyclo[1.1.1]pentan-1-yl)-5-(cyclopropylsulfonyl)furan-2-carboxamide;
N-(3-(5-chlorobenzo[d]thiazol-2-yl)bicyclo[1.1.1]pentan-1-yl)-5-((methylsulfonyl)methyl)furan-2-carboxamide;
5-(bromo(methylsulfonyl)methyl)-N-(3-(5-chlorobenzo[d]thiazol-2-yl)bicyclo[1.1.1]pentan-1-yl)furan-2-carboxamide;
N-(3-(5-chlorobenzo[d]thiazol-2-yl)bicyclo[1.1.1]pentan-1-yl)-5-(1-(methylsulfonyl)propyl)furan-2-carboxamide;
N-(3-(5-chlorobenzo[d]thiazol-2-yl)bicyclo[1.1.1]pentan-1-yl)-5-(2-(methylsulfonyl)propan-2-yl)furan-2-carboxamide;
N-[3-(5-chloro-1,3-benzothiazol-2-yl)-1-bicyclo[1.1.1]pentanyl]-5-(1-methylsulfonylethyl)furan-2-carboxamide;
N-(3-(5-chlorobenzo[d]thiazol-2-yl)bicyclo[1.1.1]pentan-1-yl)-5-(1-(methylsulfonyl)cyclopropyl)furan-2-carboxamide;
N-(3-(5-chlorobenzo[d]thiazol-2-yl)bicyclo[1.1.1]pentan-1-yl)-5-((2-methoxyethyl)sulfonyl)furan-2-carboxamide;
N-(3-(5-chlorobenzo[d]thiazol-2-yl)bicyclo[1.1.1]pentan-1-yl)-5-((methylthio)methyl)furan-2-carboxamide;
N-(3-(5-chlorobenzo[d]thiazol-2-yl)bicyclo[1.1.1]pentan-1-yl)-5-(2-(methylsulfinyl)propan-2-yl)furan-2-carboxamide;
N-[6-(5-chloro-1,3-benzoxazol-2-yl)spiro[3.3]heptan-2-yl]-1,1-dioxo-thietane-3-carboxamide;
N-[3-(5-chloro-1,3-benzothiazol-2-yl)-1-bicyclo[1.1.1]pentanyl]-2-isopropylsulfonyl-pyridine-4-carboxamide;
2-tert-butylsulfonyl-N-[3-(5-chloro-1,3-benzothiazol-2-yl)-1-bicyclo[1.1.1]pentanyl]pyridine-4-carboxamide;
N-[3-(5-chloro-1,3-benzothiazol-2-yl)-1-bicyclo[1.1.1]pentanyl]-2-(1-methylsulfonylethyl)pyridine-4-carboxamide;
N-[3-(5-chloro-1,3-benzothiazol-2-yl)-1-bicyclo[1.1.1]pentanyl]-2-(1-methyl-1-methylsulfonyl-ethyl)pyridine-4-carboxamide;
N-[3-(6-chloro-1,3-benzothiazol-2-yl)-1-bicyclo[1.1.1]pentanyl]-5-[(1R)-1-methylsulfonylethyl]furan-2-carboxamide;
N-[3-(6-chloro-1,3-benzothiazol-2-yl)-1-bicyclo[1.1.1]pentanyl]-5-[(1S)-1-methylsulfonylethyl]furan-2-carboxamide;
N-[3-(5-chloro-1,3-benzothiazol-2-yl)-1-bicyclo[1.1.1]pentanyl]-5-[(1R)-1-methylsulfonylethyl]furan-2-carboxamide;
N-[3-(5-chloro-1,3-benzothiazol-2-yl)-1-bicyclo[1.1.1]pentanyl]-5-[(1S)-1-methylsulfonylethyl]furan-2-carboxamide;
N-[3-(6-chloro-1,3-benzothiazol-2-yl)-1-bicyclo[1.1.1]pentanyl]-3-(1-methylsulfonylethyl)pyrazole-1-carboxamide;
N-[3-(6-chloro-1,3-benzothiazol-2-yl)-1-bicyclo[1.1.1]pentanyl]-3-(methylsulfonylmethyl)pyrazole-1-carboxamide;
N-[3-(6-chloro-1,3-benzothiazol-2-yl)-1-bicyclo[1.1.1]pentanyl]-3-(1-methyl-1-methylsulfonyl-ethyl)pyrazole-1-carboxamide;
N-[3-(5-bromo-1,3-benzothiazol-2-yl)-1-bicyclo[1.1.1]pentanyl]-3-(1-methyl-1-methylsulfonyl-ethyl)pyrazole-1-carboxamide;
N-[3-(5-bromo-1,3-benzothiazol-2-yl)-1-bicyclo[1.1.1]pentanyl]-3-(1-methylsulfonylcyclopropyl)pyrazole-1-carboxamide;
N-[3-(6-chloro-1,3-benzothiazol-2-yl)-1-bicyclo[1.1.1]pentanyl]-3-(1-methylsulfonylcyclopropyl)pyrazole-1-carboxamide;
N-(3-(5-chlorobenzo[d]thiazol-2-yl)bicyclo[1.1.1]pentan-1-yl)-5-(S-methylsulfonimidoyl)furan-2-carboxamide;
N-[3-(6-chloro-1,3-benzothiazol-2-yl)-1-bicyclo[1.1.1]pentanyl]-5-(cyclopropylsulfonimidoyl)furan-2-carboxamide;
N-[3-(6-chloro-1,3-benzothiazol-2-yl)-1-bicyclo[1.1.1]pentanyl]-5-(cyclopropylmethylsulfonimidoyl)furan-2-carboxamide;
N-[3-(6-chloro-1,3-benzothiazol-2-yl)-1-bicyclo[1.1.1]pentanyl]-2-(1-methylsulfonylcyclopropyl)oxazole-5-carboxamide;
N-[3-(6-chloro-1,3-benzothiazol-2-yl)-1-bicyclo[1.1.1]pentanyl]-5-(1-methylsulfonylcyclopropyl)thiazole-2-carboxamide;
N-[3-(6-chloro-1,3-benzothiazol-2-yl)-1-bicyclo[1.1.1]pentanyl]-5-(1-methylsulfonylcyclopropyl)oxazole-2-carboxamide;
N-[3-(6-chloro-1,3-benzothiazol-2-yl)-1-bicyclo[1.1.1]pentanyl]-5-(cyclopropylmethylsulfonyl)furan-2-carboxamide;
N-[3-(6-chloro-1,3-benzothiazol-2-yl)-1-bicyclo[1.1.1]pentanyl]-5-(cyclopropylmethylsulfinyl)furan-2-carboxamide;
N-[3-(6-chloro-1,3-benzothiazol-2-yl)-1-bicyclo[1.1.1]pentanyl]-5-methylsulfinyl-furan-2-carboxamide;
N-[3-(6-chloro-1,3-benzothiazol-2-yl)-1-bicyclo[1.1.1]pentanyl]-5-cyclopropylsulfinyl-furan-2-carboxamide;
N-(3-(5-chlorobenzo[d]thiazol-2-yl)bicyclo[1.1.1]pentan-1-yl)-5-(dichloro(methylsulfonyl)methyl)furan-2-carboxamide;
5-(chloro(methylsulfonyl)methyl)-N-(3-(5-chlorobenzo[d]thiazol-2-yl)bicyclo[1.1.1]pentan-1-yl)furan-2-carboxamide;
N-[3-(6-chloro-1,3-benzothiazol-2-yl)-1-bicyclo[1.1.1]pentanyl]-3-(1-methylsulfonylcyclopropyl)-1,2,4-oxadiazole-5-carboxamide;
N-[3-(6-bromo-1,3-benzothiazol-2-yl)-1-bicyclo[1.1.1]pentanyl]-5-(1-methylsulfonylcyclopropyl)-1,2,4-oxadiazole-3-carboxamide;
N-[3-(5-bromo-1,3-benzothiazol-2-yl)-1-bicyclo[1.1.1]pentanyl]-5-(1-methylsulfonylcyclopropyl)-1,2,4-oxadiazole-3-carboxamide;
5-(1-methylsulfonylcyclopropyl)-N-[3-(6-morpholino-1,3-benzothiazol-2-yl)-1-bicyclo[1.1.1]pentanyl]furan-2-carboxamide;
5-(1-methylsulfonylcyclopropyl)-N-[3-(5-morpholino-1,3-benzothiazol-2-yl)-1-bicyclo[1.1.1]pentanyl]furan-2-carboxamide;
N-[3-[6-(2-methoxyethoxy)-1,3-benzothiazol-2-yl]-1-bicyclo[1.1.1]pentanyl]-5-(1-methylsulfonylcyclopropyl)furan-2-carboxamide;
N-[3-(6-chloro-1,3-benzothiazol-2-yl)-1-bicyclo[1.1.1]pentanyl]-3-(1-methylsulfonylethyl)-1H-pyrazole-5-carboxamide;
5-(butanoylsulfamoyl)-N-[3-(6-chloro-1,3-benzothiazol-2-yl)-1-bicyclo[1.1.1]pentanyl]furan-2-carboxamide;
or a pharmaceutically acceptable salt thereof.

14. A process for the preparation of a compound according to claim 1 comprising at least one of the following steps:
(a) reaction of compound of formula (II) 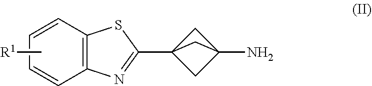
with compound of formula (III) 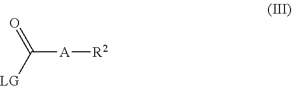
in the presence of a coupling reagent and a base,
wherein LG is OH or C₁₋₆alkoxyl, and wherein the coupling reagent is selected from HATU, T₃P and AlMe₃; the base is selected from TEA or DIPEA, or the base is absent;
(b) treatment of compound of formula (II) 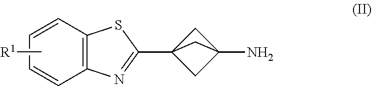
with triphosgene in the presence of a base, then reaction with compound of formula (IV) 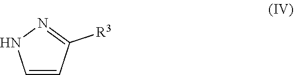
wherein R³ is C₁₋₆alkylsulfonylC₁₋₆alkyl or C₁₋₆alkylsulfonylC₃₋₇cycloalkyl, and wherein the base is TEA or DIPEA;
(c) oxidation of compound of formula (I-3) 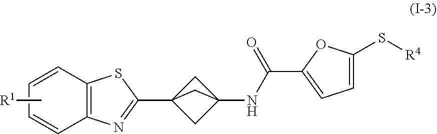
in the presence of an oxidate,
wherein R⁴ is C₁₋₆alkyl, C₃₋₇cycloalkyl, C₃₋₇cycloalkylC₁₋₆alkyl, C₁₋₆alkoxyC₁₋₆alkyl, or C₁₋₆alkylcarbonyl, and wherein the oxidate is selected from m-CPBA, or PhI(OAc)₂ and (NH₄)₂CO₃;
(d) reaction of compound of formula (I-5) 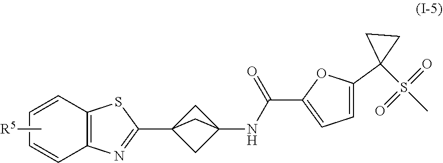
with morpholine in the presence of X-phos G3, and a base,
wherein R⁵ is halogen, and wherein the base is Cs₂CO₃;
(e) reaction of compound of formula I-7 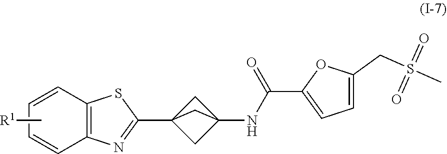
with compound CX₄ in the presence of a base,
wherein X is halogen, and wherein the base is NaOH or KOH;
(f) reaction of compound of formula 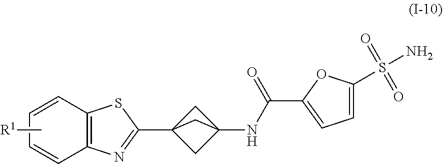
with acyl chloride (VI) 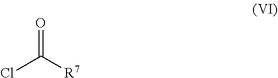
in the presence of a base,
wherein R⁷ is C₁₋₆alkyl, and wherein the base is TEA, DIPEA or K₂CO₃;
wherein R¹, R², and A are as defined in claim 1.

15. A compound or a pharmaceutically acceptable salt thereof according to claim 1 when manufactured according to the process of claim 14.

16. A pharmaceutical composition comprising a compound or a pharmaceutically acceptable salt thereof according to claim 1, and a pharmaceutically acceptable excipient.

17. A compound or a pharmaceutically acceptable salt thereof according to claim 1 for use as therapeutically active substance.

18. A compound or a pharmaceutically acceptable salt thereof according to claim 1 for use in the treatment of HBV infection.

19. The use of a compound or a pharmaceutically acceptable salt thereof according claim 1 for the treatment of HBV infection.

20. The use of a compound or a pharmaceutically acceptable salt thereof according to claim 1 for the inhibition of HBsAg.

21. The use of a compound or a pharmaceutically acceptable salt thereof according to claim 1 for the inhibition of HBeAg.

22. The use of a compound or a pharmaceutically acceptable salt thereof according to claim 1 for the preparation of a medicament for the treatment or prophylaxis of HBV infection.

23. A method for the treatment or prophylaxis of HBV infection, which method comprises administering an effective amount of a compound or a pharmaceutically acceptable salt thereof as defined in claim 1.
